(19) United States
(12) Patent Application Publication
(10) Pub. No.: US 2024/0318216 A1
(43) Pub. Date: Sep. 26, 2024

(54) Production of Human Milk Oligosaccharides in Microbial Hosts with Engineered Import/Export

(71) Applicant: Chr. Hansen HMO GmbH, Rheinbreitbach (DE)

(72) Inventors: Stefan Jennewein, Bad Honnef (DE); Dirk Wartenberg, Bonn (DE)

(73) Assignee: Chr. Hansen HMO GmbH, Rheinbreitbach (DE)

(21) Appl. No.: 18/733,787

(22) Filed: Jun. 4, 2024

Related U.S. Application Data
(62) Division of application No. 17/323,737, filed on May 18, 2021, now Pat. No. 12,037,622.
Application No. 17/323,737 is a division of application No. 15/758,653, filed on Mar. 8, 2018, now Pat. No. 11,046,985.

(30) Foreign Application Priority Data
Sep. 12, 2015 (EP) 15184968.4

(51) Int. Cl. C12P 19/18; C12N 9/10; C12N 15/70

(52) U.S. Cl. CPC C12P 19/18; C12N 9/1051; C12N 15/70; C12Y 204/01146

(57) ABSTRACT

The present invention relates to methods for the production of oligosaccharides in genetically modified bacterial host cells, as well as to the genetically modified host cells used in the methods. The genetically modified host cell comprises at least one recombinant glycosyltransferase, and at least one nucleic acid sequence coding for a protein enabling the export of the oligosaccharide.

### BACKGROUND

Human milk is regarded as the best diet for the development of infants. It is composed of fats, proteins, vitamins, minerals, trace elements and a complex carbohydrate mixture which comprises lactose and approximately 150 structurally diverse oligosaccharides (Human milk oligosaccharides, HMO).

Efforts to produce HMO chemically or by biotechnological approaches mainly attracted common attention due to their beneficial impact on the development of the gastrointestinal flora of infants, thus, advocating their use as nutritional additives. Besides these prebiotic properties, many other positive effects of HMO could be observed so far, expanding their field of application.

However, extensive scientific studies demand pure single compounds which are hardly achievable. This is especially true for complex free neutral and acidic oligosaccharides for which competitive large-scale production processes are still lacking. (e.g. lacto-N-tetraose (Gal (β1-3) GlcNAc (β1-3) Gal (β1-4) Gluc), lacto-N-neotetraose (Gal (β1-4) Glc/Ac (β1-3) Gal (β1-4) Gluc), lacto-N-fucopentaose I (Fuc (α1-2) Gal (β1-3) GlcNAc (β1-3) Gal (β1-4) Gluc) lacto-N-neofucopenaose I (Fuc (α1-2) Gal (β1-4) GlcNAc (β1-3) Gal (β1-4) Gluc) (Lacto-N-sialylpentaose a (LST-a; Neu5Ac (α2-3) Gal (β1-3) GlcNAc (β1-3) Gal (β1-4) Gluc)) The metabolic engineering of a microorganism to produce these compounds represents the most promising approach since chemical methods are rather inefficient to produce these molecules at multi-ton scale.

Several fermentative approaches were already developed for the structural simpler HMOs such as 2′-fucosyllactose, 3-fucosyllactose or 3′-sialyllactose, using mainly metabolically engineered Escherichia coli strains.

However, large-scale quantities are only achievable through boosting the oligosaccharide export out of the bacterial cell, thus, (i) enhancing the productivity and (ii) allowing the recovering of the desired oligosaccharide from the culture broth. The need for solving the export problem seems to enlarge with the size of the produced sugar. Also, with the currently available fermentation processes, upon production of more complex oligosaccharides, the problem of an unwanted export of oligosaccharide precursors from the producing cell occurs, leading to an undesirable mix of product and precursor oligosaccharides in the fermentation medium. Whereas multiple transporter proteins are known to transfer mono-or disaccharides across the membrane, hardly any knowledge exists on the transport of larger oligosaccharides (e.g., trisaccharides and larger oligosaccharides).

For example, the genome of the often used fermentation model organism E. coli encodes more than 500 distinct transporter proteins (Busch and Saier, Crit Rev Biochem Mol Biol. 2002;37 (5): 287-337). The classification of those membrane transport proteins is quite diverse and subgroups may vary in translocation mechanisms, protein structures or evolutionary origins.

Classically energy-driven active transporters perform substrate movement against its concentration or electrochemical gradient, while kinetics and direction of the substrate flow through channels primarily follows such gradients. Depending on the source of energy used for the translocation, pumps can be principally divided into primary active and secondary active transporters, exploiting metabolic energy like ATP or the electrochemical potential, respectively (Davidson and Maloney, Trends Microbiol. 2007 October;15 (10): 448-55; Forrest et al, Biochim Biophys Acta. 2011 February; 1807 (2): 167-88). Although in-depth knowledge was achieved for several membrane proteins permitting energy generation, the import of carbohydrates and the efflux of proteins and antibacterial substances, however, keen insights into mechanistic processes or information on natural or probable substrates were gained only for a minor portion of annotated bacterial transporters so far.

The E. coli lactose permease LacY probably represents the most intensively characterized solute transporter (Guan and Kaback, Annu Rev Biophys Biomol Struct. 2006;35:67-91) and is a member of the large and exceptionally diverse major facilitator superfamily (MFS)—that belongs to the secondary active transporter class—transporting sugars, drugs, hydrophobic molecules, peptides, organic ions, etc. by uniport, symport or antiport (Saier et al., J Mol Microbiol Biotechnol. 1999 November;1 (2): 257-79). Apart from a few exceptions a common structural feature of MFS transporters are two six-helical subdomains that transverse the cytoplasmic membrane. The existence of functionally homologous amino acid positions between related H⁺-coupled MFS symporters further suggests a similar kinetic mechanism as determined for the lactose permease (Madej and Kaback, Proc Natl Acad Sci USA. 2013 Dec. 10;110 (50): E4831-8).

Since decades, enormous knowledge about the import of carbohydrates into bacteria could be acquired. But regarding the export of carbohydrates, especially about molecules that are non-surface-associated, only little information is available. This is not unexpected since sugars actually depict a favourable carbon-and energy source, thus, once in the cell they shouldn't be released to a competitive environment.

However, the natural function of sugar exporters probably involve the reduction of osmotic or sugar-phosphate stress which might point to a flexible substrate spectrum. Interestingly, the export of a variety of galactosides like IPTG, TMG and lactose was shown for members of the so called sugar efflux transporter family (SET), which belong to the group of MFS transporters (Liu et al., J Biol Chem. 1999 Aug. 13;274 (33): 22977-84; Liu et al., Mol Microbiol. 1999 March;31 (6): 1845-51).

The E. coli transport protein SetA was even described to transfer the human milk oligosaccharide 3-fucosyllactose resulting in an improved production of said compound during fermentation of a recombinant E. coli strain overexpressing setA (see applicant's international patent application WO 2010/142305). Similarly, the expression of a sugar efflux transporter from Yersinia was shown to enable the export of the human milk oligosaccharide 2′-fucosyllactose out of an engineered E. coli production strain.

Apart from this, from a mechanistic and energetic point of view, only the ion-gradient-driven transport systems have the potential to translocate solutes in both directions across the membrane. This is exemplarily true for the above mentioned LacY, a galactoside/H⁺ symporter, which is part of the bacterial lac operon that allows the metabolism of lactose in E. coli. This permease primarily imports lactose into the cell but it is also capable to transfer its substrate in the opposite direction.

Besides the major facilitator superfamily, which represents the largest group of transporters, bacteria possess further mechanisms to excrete solutes-often summarized in the classes of multidrug efflux pumps. Alike for the MFS, the activities of the small multidrug resistance superfamily (SMR), the multidrug and toxic compound extrusion superfamily (MATE) and the resistance-nodulation-cell division superfamily (RND) rely on the electrochemical gradient. The fifth class is the adenosine triphosphate (ATP)-binding cassette superfamily (ABC) which uses ATP as energy source to drive molecules from the cell. As for the MFS, members of SMR, MATE, RND and ABC transport structurally diverse molecules. Further, most of their so far identified substrates are not naturally occurring, and, thus, their preferences are hardly predictable.

Although chemical synthesizing processes are known for human milk oligosaccharides, these processes are very cost-intensive and do not lead to satisfying amounts. On the other hand, fermentation processes using genetically modified microorganisms still have the drawback that the export of larger oligosaccharides (tetra-, penta-, hexasaccharides) represents a major limitation for the establishment of cost effective production processes. As a consequence, there still is the need for improved processes for the production of large-scale human oligosaccharides.

### SUMMARY OF THE INVENTION

According to the invention, this and other objects are solved by the methods and microbial host cell(s) as claimed in the attached claims.

With the methods and host cells according to the invention it is possible to produce a desired oligosaccharide, preferably an oligosaccharide that is not produced in an unmodified host cell, and also preferably an oligosaccharide belonging to the human milk oligosaccharides, in large amounts obtainable from the medium. As such, the oligosaccharide is, so to say, obtainable in free from in the medium; it is not bound to a surface protein or membrane protein or other protein of the surface of the host cell.

According to the invention, a method for the production of a desired oligosaccharide by a genetically modified microbial host cell, comprising the steps of a) providing a genetically modified microbial host cell that comprises at least one recombinant glycosyltransferase, and that has the expression or activity of at least one endogenous sugar export protein modified such, that the expression or activity of the sugar export protein is either (i) increased or (ii) decreased or inactivated as compared to an genetically unmodified host cell, so that (i) the export of a oligosaccharide into the medium is either decreased or abolished, or (ii) the transport of a desired oligosaccharide is increased, respectively, as compared to an genetically unmodified host cell, b) cultivating the host cell in a medium under conditions permissive for the production of the desired oligosaccharide, whereby the desired oligosaccharide is transported into the medium. The method may further comprise the step of c) obtaining the desired oligosaccharide from the medium.

In the method according to the invention, it is preferred if the desired oligosaccharide is a human milk oligosaccharide comprising a lacto-N-triose II (LNT-II; GlcNAc (β1-3) Gal (β1-4) Gluc) as a core trisaccharide. In this connection, an oligosaccharide having a “core trisaccharide” is meant to comprise the specific trisaccharide representing the reducing end of a desired oligosaccharide, and comprising, as the case may be, additional saccharide moieties, with the specific trisaccharide representing the major moiety.

Accordingly, in an embodiment of the method and the host cell according to the invention, the desired oligosaccharide is selected from the group consisting of: lacto-N-triose II, lacto-N-tetraose, lacto-N-neotetraose, lacto-N-fucopentaose I, lacto-N-fucopentaose II, lacto-N-fucopentaose III, lacto-N-fucopentaose V, lacto-N-difucosylhexose I, lacto-N-difucosylhexaose II, lacto-N-sialylpentaose LSTa, LSTb, LSTc, disialyllacto-N-tetraose, disialyllacto-N-neotetraose.

In order to overcome the above mentioned drawbacks of limited oligosaccharide export the object is further solved by a method according to the invention, wherein the host cell comprises: at least one homologous or heterologous nucleic acid sequence coding for a protein enabling the export of a desired oligosaccharide into the culture medium, wherein said host cell has been modified such, that the expression of the homologous or heterologous nucleic acid sequence is overexpressed or under control of a promoter enabling the overexpression of the nucleic acid sequence; and/or the deletion, disruption, diminishment or inactivation of at least one endogenous nucleic acid sequence coding for an exporter protein that exports precursors of the desired oligosaccharide outside the host cell; and/or at least one homologous or heterologous nuclei acid sequence coding for a protein mediating the import of a precursor of a desired oligosaccharide into said host cell, wherein preferably the nucleic acid sequence is overexpressed, and wherein preferably the precursor is larger than a disaccharide.

The genetically modified microbial host cell comprising the characteristics as set forth herein are cultured in the presence of glucose, sucrose, glycerin or a combination thereof—using these substrates as carbon-and energy sources—as well as in the presence of lactose or oligosaccharides larger than disaccharides, e.g., LNT-II.

In a preferred embodiment of this method and host cell, said protein enabling the export of a desired oligosaccharide belongs to the class of secondary active transporters, and more preferably effects the export of an oligosaccharide comprising at least three moieties.

According to preferred embodiments, for the export of desired oligosaccharides a suitable exporter is expressed in addition to the genes that are responsible for intracellular oligosaccharide biosynthesis.

According to one aspect of the method and host cell of the invention, the at least one nucleic acid sequence coding for a protein enabling the export of a desired oligosaccharide is an endogenous or a recombinant nucleic acid.

In a preferred embodiment of the method and the host cell of the invention, the nucleic acid sequence coding for a protein enabling the export of a desired oligosaccharide is of bacterial, archeal, plant, yeast or animal origin; preferably, the at least one nucleic acid sequence coding for a protein enabling the export of a desired oligosaccharide is a gene selected from the group consisting of yebQ and yjhB from Escherichia coli, proP from Mannheimia succiniciproducens and setA from Cedecea neteri or functional fragments thereof.

Preferably, the oligosaccharide exporter is a protein selected from at least one of the following: SetA, SetB, SetC, YdeA, Cmr, YnfM, MdtD, YfcJ, YhhS, EmrD, YdhC, YbdA, YdeE, MhpT, YebQ, YjhB, Bcr and YdeA of E. coli, or ProP from Mannheimia succiniciproducens and SetA from Cedecea neteri or variants or homologs thereof.

In yet another preferred embodiment, the recombinant glycosyltransferase is selected from at least one of the following: a galactosyltransferase, a sialyltransferase, an N-acetylglucosaminyltransferase and a fucosyltransferase, and is preferably selected from at least one of the following: β-1,3-N-acetylglucosaminyltransferase, β-1,3-galactosyltransferase, β-1,4-galactosyltransferase, β-1,6-galactosyltransferase, α-2,3-sialyltransferase, α-2,6-sialyltansferase, α-1,2-fucosyltransferase, or α-1,3-fucosyltransferase.

A preferred embodiment of the method and the host cell of the invention, concerns the a host cell or its provision, wherein the host cell comprises (i) a β-1,3-N-acetylglucosaminyltransferase, and (ii) a β-1,3-galactosyltransferase or a β-1,4-galactosyltransferase as glycosyltransferases. In this connection it is preferred, if said β-1,3-N-acetylglucosaminyltransferase has the activity of ligating N-acetylglucosamine to lactose generating lacto-N-triose II, and if said β-1,3-galactosyltransferase or said β-1,4-galactosyltransferase, respectively, have the activity to galactosylate lacto-N-triose II thus generating lacto-N-tetraose or lacto-N-neotetraose, respectively. The here developed system is easily adaptable to even more complex oligosaccharides by the expression of further glycosyltransferases.

With the microbial cell and the method according to the invention, it is possible to ferment a desired oligosaccharide in large quantities, especially an oligosaccharide comprising LNT-II as core structure, and to recover it from the culture broth.

In a preferred embodiment, said β-1,3-N-acetylglucosaminyltransferase belongs to the class of IgtA of Neisseria meningitides or PmnagT of Pasteurella multocida, or variants thereof.

Preferably, the glycosyltransferase is selected from a galactosyltransferase, a sialyltransferase, an N-acetylglucosaminyltransferase and a fucosyltransferase.

In yet another preferred embodiment, the lacto-N-tetraose generating β-1,3-galactosyltransferase is WbdO or a functional variant thereof. According to an aspect of the invention, the B-1,3-galactosyltransferase is a B-1,3-galactosyltransferase derived from Salmonella enterica (wbdO, acc. no. AY730594), and is preferably encoded by a gene selected from the group consisting of wbgO from Escherichia coli 055: H7 or furA from Lutiella nitroferrum, or a functional fragments thereof.

The invention also concerns a genetically modified microbial host cell, preferably a bacterial host cell, as described above in which the endogenous β-galactosidase gene is inactivated or deleted and in which a functional lactose permease gene is present.

Accordingly, in a preferred embodiment of the method and the host cell of the invention, a genetically modified host cell is provided, in which, where applicable, an endogenous β-galactosidase gene and a glucosamine-6-phosphate deaminase gene are inactivated or deleted, and wherein said genetically modified host cell comprises a nucleic acid sequence coding for a functional lactose permease protein, preferably LacY.

In a preferred embodiment, the genetically modified host cell comprises an increased UDP-N-acetylglucosamine and UDP-galactose, GDP-fucose or CMP-N-acetylneuraminic acid production capability as compared to a genetically unmodified host cell.

In a refinement of this embodiment of he method of and of the host cell of the invention, said increased UDP-N-acetylglucosamine and UDP-galactose production capability comprises the overexpression of one or more genes encoding for proteins comprising the following activities for a: L-glutamine: D-fructose-6-phosphate aminotransferase, N-acetyl glucosamine-1-phosphate uridyltransferase/glucosamine-1-phosphate acetyl transferase, phosphoglucosamine mutase, UDP-galactose-4-epimerase, phosphoglucomutase, glucose-1-phosphate uridylyltransferase.

For the synthesis of, e.g. LNT, UDP-galactose and UDP-N-acetylglucosamine are required. UDP-galactose can be obtained by feeding galactose to the HMO producing bacterial host cell via the fermentation medium. The galactose is then taken up by the cell, phosphorylated to galactose-1-phosphate and then converted to UDP-galactose. Genes encoding these enzymatic activities are well known in the literature (Grossiord et al., J. Bacteriol 2003 185 (3) 870-878). The supply for UDP-galactose can be also obtained from the cells own metabolism, and the metabolism can be improved by further genetic modification, such as the overexpression of the UDP-galactose-4′-epimerase, or the UDP-galactose-4′-epimerase in combination with the glucose-1-phosphate-1-uridinyltransferase. UDP-N-acetlyglucosamine can be also obtained from the bacterial host cell's own UDP-N-acetylglucosamine metabolism. The provision of UDP-N-acetylglucosamine for the synthesis of N-aectylglucosamine containing oligosaccharides can be improved by the inactivation of the N-acetylglucosamine catabolism within the producing cell.

According to one aspect of the invention, the genetically modified host cell is cultivated in the presence of glucose, sucrose, glycerol or a combination thereof, but neither by addition or in the presence of N-acetylglucosamine or galactose nor in a combination thereof.

In a preferred embodiment of the method and of the host cell of the invention, the desired oligosaccharide is lacto-N-triose II, which is produced by total fermentation from a simple carbon source in the host cell by the action of the heterologous expressed glycosyltransferases β-1,4-galactosyltransferase and β-1,3-N-acetylglucosaminyltransferase.

The present invention, as already mentioned above, also concerns a genetically modified host cell for the production of a desired oligosaccharide, the oligosaccharide comprising a lacto-N-triose II (LNT-II; GlcNAc (β1-3) Gal (β1-4) Gluc) as a core trisaccharid, wherein the host cell comprises at least one recombinant glycosyltransferase, the glycosyltransferase being preferably selected from a galactosyltransferase, a sialyltransferase, and an N-acetylglucosaminyltransferase, and has the expression or activity of at least endogenous sugar transport protein modified such, that the expression or activity of the endogenous sugar transport protein is functionally inactivated for the export of a precursor of the desired oligosaccharide.

A preferred embodiment concerns a host cell as described above, comprising (i) a heterologous expressed β-1,3-N-acetylglucosaminyltransferase, and (ii) a heterologous expressed β-1,3-galactosyltransferase or a heterologous expressed β-1,4-galactosyltransferase as glycosyltransferases, wherein the host cell further preferably comprises at least one homologous or heterologous nucleic acid sequence coding for a protein enabling the export of the oligosaccharide into a culture medium the host cell is cultivated in, wherein said protein enabling the export of the desired oligosaccharide belongs to the class of secondary active transporters, wherein said host cell has been modified such, that the expression of the homologous or heterologous nucleic acid sequence is overexpressed or under control of a promoter enabling the overexpression of the nucleic acid sequence. In preferred embodiments of the host cell, said at least one nucleic acid sequence coding for a protein enabling the export of the desired oligosaccharide is an endogenous or a recombinant nucleic acid sequence.

As already outlined for the method according to the invention, it is also preferred in the host cell of the invention, if said nucleic acid sequence coding for a protein enabling the export of a desired oligosaccharide is of bacterial, archeal, plant, yeast or animal origin.

According to another aspect of the invention, the host cell as described above further comprises: the deletion, disruption, diminishment or inactivation of at least one endogenous nucleic acid sequence coding for an exporter protein that exports precursors of the desired oligosaccharide outside the host cell; and/or at least one homologous or heterologous nucleic acid sequence coding for a protein enabling the import of a precursor of a desired oligosaccharide into said host cell, wherein preferably the nucleic acid sequence is overexpressed, and wherein preferably the precursor is larger than a disaccharide.

With the overexpression of at least one homologous or heterologous nucleic acid sequence coding for a protein enabling the import of a precursor of a desired oligosaccharide into said host cell, it is possible to feed precursors of a desired oligosaccharide to the culture medium, which get imported into the host cell, such as, e.g., LNT-II.

According to one aspect of the invention, in the host cell said at least one nucleic acid sequence coding for a protein enabling the export of a desired oligosaccharide is a gene selected from the group consisting of yebQ and yjhB from Escherichia coli, proP from Mannheimia succiniciproducens and setA from Cedecea neteri or functional fragments thereof.

According to yet another preferred embodiment, the desired oligosaccharide is lacto-N-triose II, and the protein enabling the export of the oligosaccharide into a culture medium the host cell is cultivated in, is YjhB from Escherichia coli, ProP from Mannheimia succiniciproducens and SetA from Cedecea neteri or functional fragments thereof.

According to a preferred embodiment, the microbial host according to the invention is further modified not to express proteins exporting precursors of a desired oligosaccharide.

In a preferred embodiment of the host cell, the desired oligosaccharide is lacto-N-tetraose, the precursor is lacto-N-triose II, and the host cell has deleted, disrupted or inactivated at least one nucleic acid sequence coding for an exporter protein that is able to export lacto-N-triose II outside the host cell.

In this connection it is preferred, if the protein enabling the export of lacto-N-tetraose is selected from YebQ from Escherichia coli BL21 (DE3), SpoVB of Bacillus amyloliquefaciens, YabM of Erwinia pyrilfolia, Bcr of E. coli MG1655, YdeA of E. coli MG1655, ProP2 of Haemophilus parainfluenzae, SetA of Pectobacterium carotovorum, FucP of E. coli MG1655, MdeA of Staphylococcus aureus Bmb9393, ImrA of Lactococcus lactis, SetA of Pseudomonas sp. MT-1 and SetA of Beauveria bassiana D1-5.

Preferably, the oligosaccharide exporter is a protein selected from at least one of the following: SetA, SetB, SetC, YdeA, Cmr, YnfM, MdtD, YfcJ, YhhS, EmrD, YdhC, YbdA, YdeE, MhpT, YebQ, YjhB, Bcr and YdeA of E. coli, or ProP from Mannheimia succiniciproducens and SetA from Cedecea neteri or variants or homologs thereof.

Presently, the term “nucleic acid” refers to a single-or double-stranded deoxyribonucleotide or ribonucleotide macromolecule and encompasses known analogues or natural or synthetically produced nucleotides that hybridize with the desired nucleic acid and that encode a certain polypeptide.

The term “recombinant” or “genetically modified”, as used herein with reference to a microbial host cell indicates that the microbial host cell replicates a heterologous nucleic acid, or expresses a peptide or protein encoded by a heterologous nucleic acid (i.e., a sequence “foreign to said cell”). Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques. Accordingly, a “recombinant polypeptide” is one which has been produced by a recombinant cell. A “heterologous sequence” or a “heterologous nucleic acid”, as used herein, is one that originates from a source foreign to the particular host cell (e.g. from a different species), or, if from the same source, is modified from its original form. Thus, a heterologous nucleic acid operably linked to a promoter is from a source different from that from which the promoter was derived, or, if from the same source, is modified from its original form. The heterologous sequence may be stably introduced, e.g. by transfection, transformation, conjugation or transduction, into the genome of the host microbial host cell, thus representing a genetically modified host cell. Techniques may be applied which will depend on the host cell the sequence is to be introduced. Various techniques are known to a person skilled in the art and are, e.g., disclosed in Sambrook et al., Molecular Cloning: A Laboratory Manual, 2nd Ed., Cold Spring Harbor Laboratory Press, Cold Spring Harbor, N.Y. (1989).

Accordingly, a “microbial host cell” is presently understood as a microbial, preferably bacterial, cell which has been transformed or transfected, or is capable of transformation or transfection by an exogenous polynucleotide sequence.

Thus, the nucleic acid sequences as used in the present invention, may, e.g., be comprised in a vector which is to be stably transformed/transfected or otherwise introduced into host microorganism cells.

Presently, the term “operably linked” as used herein, shall mean a functional linkage between a nucleic acid expression control sequence (such as a promoter, signal sequence, or array of transcription factor binding sites) and a second nucleic acid sequence, wherein the expression control sequence affects transcription and/or translation of the nucleic acid corresponding to the second sequence. Accordingly, the term “Promoter” designates DNA sequences which usually “precede” a gene in a DNA polymer and provide a site for initiation of the transcription into mRNA. “Regulator” DNA sequences, also usually “upstream” of (i.e., preceding) a gene in a given DNA polymer, bind proteins that determine the frequency (or rate) of transcriptional initiation. Collectively referred to as “promoter/regulator” or “control” DNA sequence, these sequences which precede a selected gene (or series of genes) in a functional DNA polymer cooperate to determine whether the transcription (αnd eventual expression) of a gene will occur. DNA sequences which “follow” a gene in a DNA polymer and provide a signal for termination of the transcription into mRNA are referred to as transcription “terminator” sequences.

A great variety of expression systems can be used to produce the polypeptides of the invention. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses, and vectors derived from combinations thereof, such as those derived from plasmid and bacteriophage genetic elements, such as cosmids and phagemids. The expression system constructs may contain control regions that regulate as well as engender expression. Generally, any system or vector suitable to maintain, propagate or express polynucleotides and to synthesize a polypeptide in a host may be used for expression in this regard. The appropriate DNA sequence may be inserted into the expression system by any of a variety of well-known and routine techniques, such as, for example, those set forth in Sambrook et al., supra.

The art is rich in patent and literature publications relating to “recombinant DNA” methodologies for the isolation, synthesis, purification and amplification of genetic materials for use in the transformation of selected host organisms. Thus, it is common knowledge to transform host organisms with “hybrid” viral or circular plasmid DNA which includes selected exogenous (i.e. foreign or “heterologous”) DNA sequences. The procedures known in the art first involve generation of a transformation vector by enzymatically cleaving circular viral or plasmid DNA to form linear DNA strands. Selected foreign DNA strands usually including sequences coding for desired protein product are prepared in linear form through use of the same/similar enzymes. The linear viral or plasmid DNA is incubated with the foreign DNA in the presence of ligating enzymes capable of effecting a restoration process and “hybrid” vectors are formed which include the selected exogenous DNA segment “spliced” into the viral or circular DNA plasmid.

As used herein, the term “cultivating” means growing a bacterial cell in a medium and under conditions permissive and suitable for the production of the desired oligosaccharide(s). A couple of suitable bacterial host cells as well as mediums and conditions for their cultivation will be readily available for one skilled in the art upon reading the disclosure of this invention in connection with the skilled person's technical and expert background.

As used herein, the term “recovering” or “obtaining” means isolating, harvesting, purifying, collecting or otherwise separating from the host cell culture the oligosaccharide produced by the host cell according to the invention.

A “microbial” host cell according to the invention, and as generally understood, means any microorganism, including bacteria, fungi and archaea, which is generally suitable for cultivation in large amounts, and which can be genetically modified according to the invention in order to produce a desired oligosaccharide. Preferred microorganisms are bacteria, e.g. Escherichia coli, Corynebacterium glutamicum and the yeast Saccharomyces sp., which have the advantage that these microorganisms can be grown easily and inexpensively in laboratory settings, and the bacteria and yeast have been intensively investigated for over many years

Generally, and throughout the present invention, the term “glycosyltransferase activity” or “glycosyltransferase” designates and encompasses enzymes that are responsible for the biosynthesis of disaccharides, oligosaccharides and polysaccharides, and they catalyze the transfer of monosaccharide moieties from an activated nucleotide monosaccharide/sugar (the “glycosyl donor”) to a glycosyl acceptor molecule.

Generally, and throughout the present invention, the terms “exporter” or “exporter protein” or “protein enabling the export of a desired oligosaccharide”, which terms are presently being used synonymously, designates one or more polypeptides that solely or as part of a multi-protein complex transfers an oligosaccharide from the intracellular milieu of a bacterial cell into the periplasm of said cell or the culture supernatant, thus, enabling the oligosaccharide to pass the cellular membrane and/or the cell wall of said cell.

Within the scope of the present invention, also nucleic acid/polynucleotide and polypeptide polymorphic variants, alleles, mutants, and interspecies homologs are comprised by those terms, that have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 25, 50, 100, 200, 500, 1000, or more amino acids, to a polypeptide encoded by a wild type glycosyltransferase activity or oligosaccharide export displaying protein.

“Variant(s)” as the term is used herein, is a polynucleotide or polypeptide that differs from a reference polynucleotide or polypeptide, respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a poly-peptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to the persons skilled in the art.

Accordingly, a “functional fragment” of any of the genes/proteins disclosed therein, is meant to designate sequence variants of the genes/proteins still retaining the same or somewhat lesser activity of the gene or protein the respective fragment is derived from.

In this connection, the term “nucleic acid sequence encoding . . . ” generally refers to any polyribonucleotide or polydeoxyribonucleotide, which may be unmodified RNA or DNA or modified RNA or DNA, and generally represents a gene which encodes a certain polypeptide or protein.

In this context, the term “polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as “proteins”. Polypeptides may contain amino acids other than the 20 gene encoded amino acids. “Polypeptide(s)” include those modified either by natural processes, such as processing and other post-translational modifications, but also by chemical modification techniques. It will be appreciated that the same type of modification may be present in the same or varying degree at several sites in a given polypeptide, without essentially altering the activity of the polypeptide. Also, a given polypeptide may contain many types of modifications. Modifications can occur anywhere in a polypeptide, including the peptide backbone, the amino acid side-chains, and the amino or carboxyl termini.

Further, with the expression “precursor” compounds are encompassed which are involved in the biosynthetic pathway of the oligosaccharide according to the invention or which are produced and naturally present in the host cell.

A “precursor that is larger than a disaccharide” is presently understood as a sugar moiety that comprises more than two monosaccharide residues.

The term “desired oligosaccharide” refers to a sugar polymer consisting of at least three moieties, thus, comprising trisaccharides, tetrasaccharides, pentasaccharides etc., preferably an oligosaccharide selected from at least one of the following: lacto-N-triose II, lacto-N-tetraose, lacto-N-neotetraose, lacto-N-fucopentaose I, lacto-N-fucopentaose II, lacto-N-fucopentaose III, lacto-N-fucopentaose V, lacto-N-difucosylhexose I, lacto-N-difucosylhexaose II, lacto-N-sialylpentaose LSTa, LSTb, LSTc, disialyllacto-N-tetraose, disialyllacto-N-neotetraose.

Presently, and as generally understood in the relevant field, the expression “homologous” refers to a nucleic acid sequence that encodes for a specific product or products and is derived from the same species, in which said nucleic acid sequence is inserted. Accordingly, the term “heterologous” refers to a nucleic acid sequence encoding for a specific product or products and being derived from a species other than those in which said nucleic acid sequence is inserted.

The term “endogenous” herein and generally within the field means that the nucleic acid encoding for an enzyme of interest is originating from the bacterial host cell and has not been introduced into said host cell, whereas a “recombinant” nucleic acid has been introduced into said host cell and does not originates from said host cell.

The expression “overexpressed”, or “overexpressing” or “under control of a promoter sequence enabling the overexpression of said nucleic acid sequence” presently, and generally in the art, means the expression of a gene in greater-than-normal amounts, i.e. in increased quantity thus leading to an increased amount of the protein the nucleic acid sequence is coding for.

In some embodiments, the nucleic acid sequence is placed under the control of an inducible promoter, which is a promoter that directs expression of a gene where the level of expression is alterable by environmental or developmental factors such as, for example, temperature, pH, anaerobic or aerobic conditions, light, transcription factors and chemicals. Such promoters are referred to herein as “inducible” promoters, which allow one to control the timing of expression of the proteins used in the present invention. For E. coli, and other microbial host cells, inducible promoters are known to those of skill in the art.

Further advantages are evident from the description and the drawings.

It is understood that the features mentioned above and those yet to be explained below can be used not only in the respective combinations indicated, but also in other combinations or in isolation, without leaving the context of the present invention.

The invention will be described in more detail in the examples and the attached figures, in which

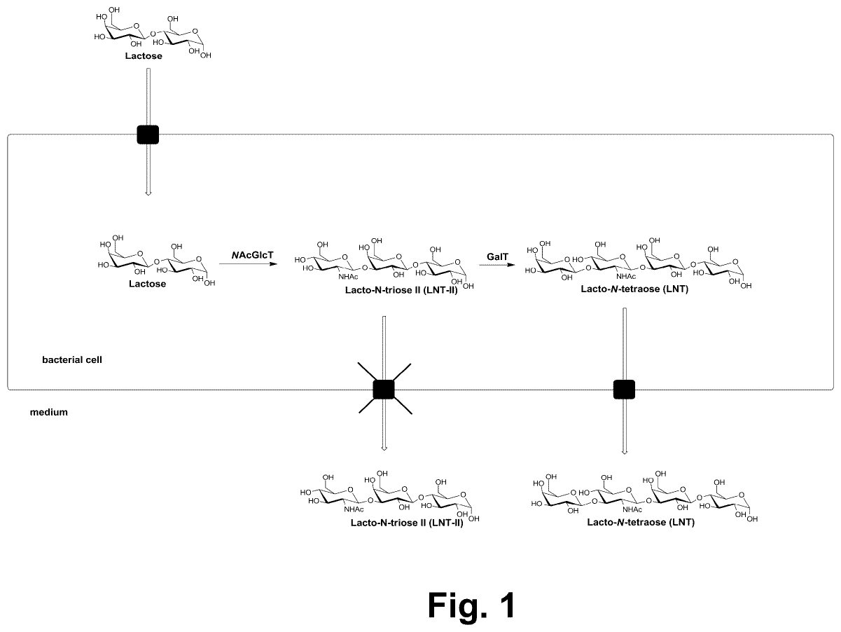

FIG. 1 shows a schematic illustration for the production of either lacto-N-triose II or lacto-N-tetraose in a host cell cultivated in a medium;.

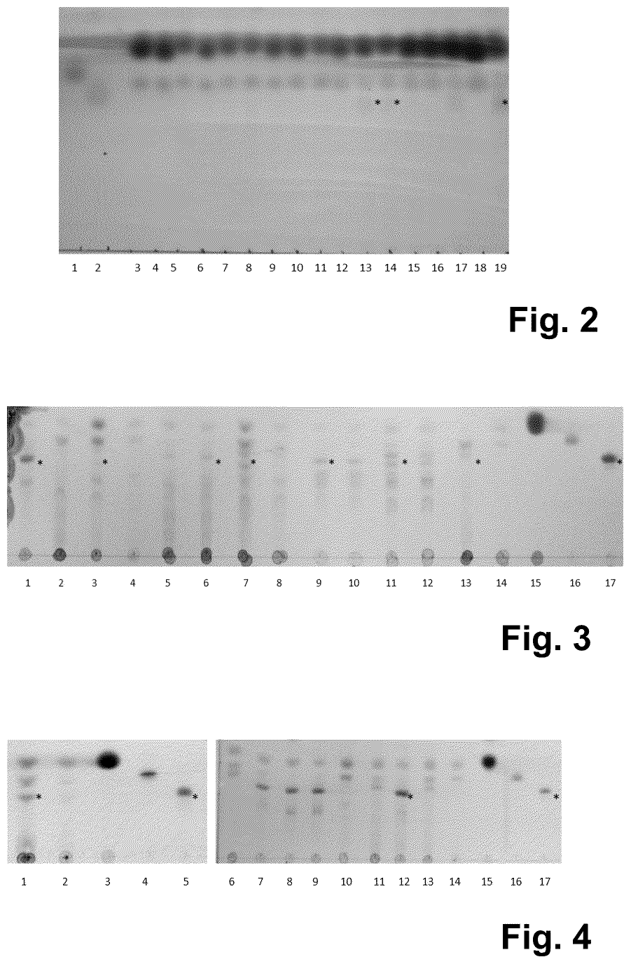

FIG. 2 shows the results of the TLC analysis of culture extracts of lacto-N-triose II (LNT II) producing E. coli BL21 (DE3) strains overexpressing the β-1,3-N-acetyl glucosaminyltransferase gene PmnagT (13, 14);

FIG. 3 shows the results of the TLC analysis of culture extracts of lacto-N-tetraose (LNT) producing E. coli BL21 (DE3) strains overexpressing the β-1,4-galactosyltransferase encoding genes BfgalT2 (1), PmgalT7 (3), MsgalT8 (6), gatD (7), lex1 (9), lgtB (11) or IsgD (13);

FIG. 4 shows the results of the TLC analysis of culture extracts of lacto-N-tetraose (LNT) producing E. coli BL21 (DE3) strains overexpressing the β-1,4-galactosyltransferase encoding genes KdgalT10 (1), cpsl14J (7), cpslaJ (8, 9), HpgalT (12);

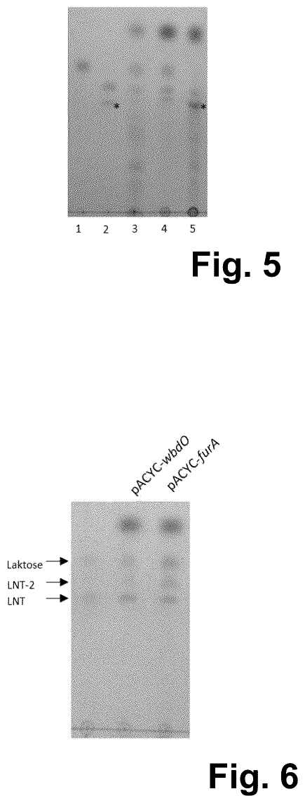

FIG. 5 shows the results of the TLC analysis of culture extracts of lacto-N-tetraose producing E. coli BL21 (DE3) strains overexpressing the β-1,4-galactosyltransferase encoding gene waaX (5);

FIG. 6 shows the results of the TLC analysis of culture extracts of lacto-N-tetraose producing E. coli BL21 (DE3) strains overexpressing the β-1,3-galactosyltransferase encoding genes wbdO or furA;

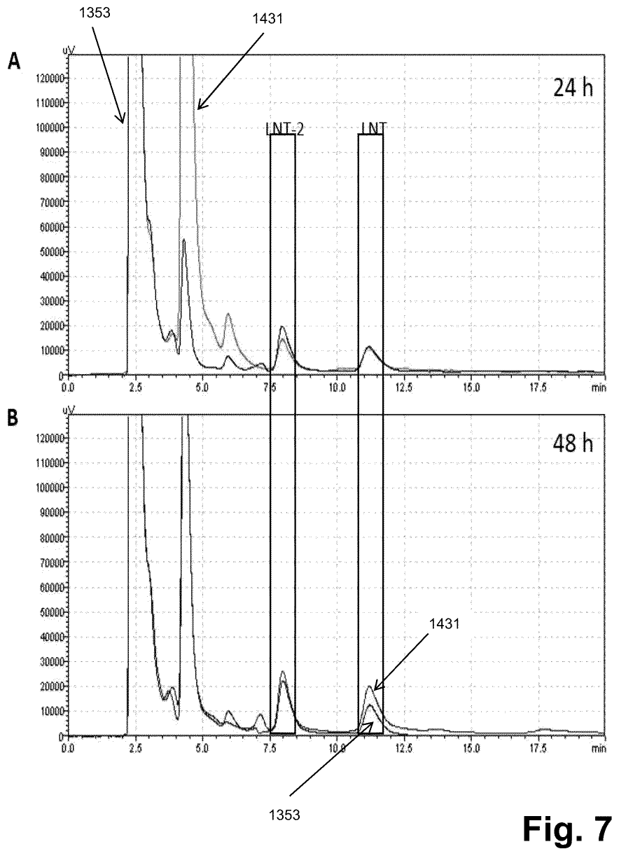

FIG. 7 shows the results of HPLC analyses of the culture supernatant of lacto-N-tetraose producing E. coli BL21 (DE3) strain. (A) Supernatant of E. coli BL21 (DE3) 1353 and 1431 grown in the presence of glucose and lactose after 24 h of incubation. (β) Supernatant of E. coli BL21 (DE3) 1353 and 1431 grown in the presence of glucose and lactose after 48 h of incubation;

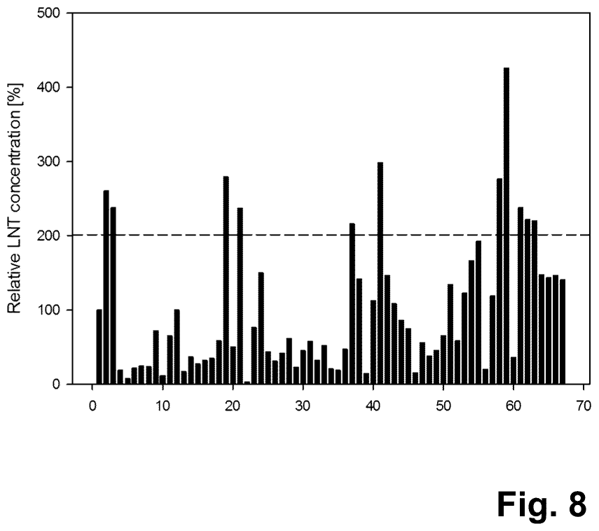

FIG. 8 shows a diagram depicting the relative concentration of lacto-N-tetraose in the supernatant of E. coli BL21 (DE3) strains overexpressing sugar efflux transporters compared to the control strain 1353; and

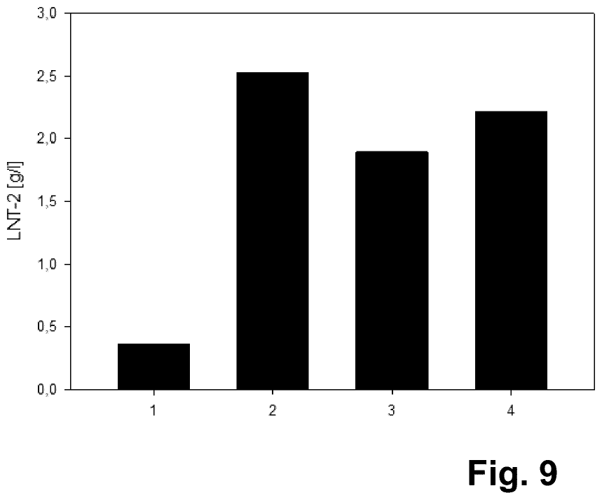

FIG. 9 shows a diagram depicting concentrations of lacto-N-triose II in the supernatant of E. coli BL21 (DE3) strains overexpressing the sugar efflux transporters TP11 (2), YjhB (3) or TP70 (4).

### EXAMPLES

FIG. 1 shows a schematic drawing of an exemplary host cell 10 according to the invention, importing lactose and synthesizing lacto-N-triose II (LNT II) and lacto-N-tetraose (LNT). Lactose is imported from the medium the host cell is cultivated in into the cell via transporter 1. The enzyme N-acetylglucosaminyltransferase NacGlcT ligates N-acetylglucosamine to the acceptor substrate lactose, thus generating LNT-II. LNT-Il is exported from the cell via exporter protein 20. Since LNT-II is a precursor of LNT or LNnT, the exporter exporting LNT-II represents an exporter protein exporting precursors of the latter oligosaccharides. As can further be seen from FIG. 1, the cell comprises a protein having β-1,3-galactosyltransferase activity enabling the galactosylation of LNT-II to intracellularly generate LNT; the cell may also and/or alternatively comprise or β-1,4-galactosyltransferase activity enabling the galactosylation of LNT-II to intracellularly generate lacto-N-neotetraose LNnt. LNT-or as the case may be LNnt-is then exported, via a oligosaccharide exporter from the cell into the culture medium the cell is cultivated in.

The exporters are membrane-bound, and their expression can be either overexpressed, which-in case of overexpression of the LNT-II exporter leads to an increased LNT-II export and to a decreased LNT export, whereas when the LNT-II exporting exporter protein is deleted or otherwise inactivated, this leads to an improved LNT-export. The LNT-II exporter preferably is an endogenous exporter protein, whereas the LNT-exporter protein preferably is a heterologous exporter protein.

### Example 1

Development of an E. coli Lacto-N-triose II Production Strain

Escherichia coli BL21 (DE3) was used to construct a lacto-N-triose II (LNT-2) producing strain. Metabolic engineering included mutagenesis and deletions of specific genes, respectively, and genomic integrations of heterologous genes. The genes lacZ and araA were inactivated by mutagenesis using mismatch-oligonucleotides as described by Ellis et al., “High efficiency mutagenesis, repair, and engineering of chromosomal DNA using single-stranded oligonucleotides”, Proc. Natl. Acad. Sci. USA 98:6742-6746 (2001).

Genomic deletions were performed according to the method of Datsenko and Warner (Proc. Natl. Acad. Sci. USA 97:6640-6645 (2000)). To prevent intracellular degradation of N-acetylglucosamine, genes encoding N-acetylglucosamine-6-phosphate deacetylase (nagA) and glucosamine-6-phosphate deaminase (nagB) were deleted from the genome of the E. coli strain BL21 (DE3) strain. Also genes wzxC-wcaJ were deleted. WcaJ encodes an UDP-glucose: undecaprenyl phosphate glucose-1-phosphate transferase catalysing the first step in colanic acid synthesis (Stevenson et al., J. Bacteriol. 1996, 178:4885-4893). In addition the genes fucI and fucK, coding for L-fucose isomerase and L-fuculose kinase, respectively, were removed.

Genomic integration of heterologous genes was performed by transposition. Either the EZ-Tn5™ transposase (Epicentre, USA) was used to integrate linear DNA-fragments or the hyperactive C9-mutant of the mariner transposase Himar1 (Lampe et al., Proc. Natl. Acad. Sci. 1999, USA 96:11428-11433) was employed for transposition. To produce EZ-Tn5 transposomes the gene of interest together with a FRT-site flanked antibiotic resistance marker was amplified with primer 1119 and 1120 (αll primer used are listed in table 3 below); the resulting PCR-product carried on both sites the 19-bp Mosaic End recognition sites for the EZ-Tn5 transposase. For integration using Himar1 transposase expression constructs (operons) of interest were similarly cloned together with a FRT-site flanked antibiotic resistance marker into the pEcomar vector. The pEcomar vector encodes the hyperactive C9-mutant of the mariner transposase Himar1 under the control of the arabinose inducible promoter ParaB. The expression fragment <Ptet-lacY-FRT-aadA-FRT> (SeqID1) was integrated by using the EZ-Tn5 transposase. After successful integration of the gene for the lactose importer LacY from E. coli K12 TG1 acc. no. ABN72583) the resistance gene was eliminated from streptomycin resistant clones by the FLP recombinase encoded on plasmid pCP20 (Datsenko and Warner, Proc. Natl. Acad. Sci. 2000, USA 97:6640-6645). The N-acetylglucosaminyltransferase gene IgtA from Neisseria meningitidis MC58 (αcc. no. NP_274923) was codon-optimized for expression in E. coli and prepared synthetically by gene synthesis. Together with the gene galT, encoding a galactose-1-phosphate uridylyltransferase from E. coli K-12 substr. MG1655 (acc. no. NP_415279) that was similarly obtained by gene synthesis, IgtA was inserted by transposition (SeqID2) using plasmid pEcomar-IgtA-galT. To enhance de novo synthesis of UDP-N-acetylglucosamine, genes encoding L-glutamine: D-fuctose-6-phosphate aminotransferase (glmS), phosphoglucosamine mutase from E. coli K-12 substr. MG1655 (glmM) and N-acetylglucosamine-1-phosphate uridyltransferase/glucosamine-1-phosphate acetyltransferase (glmU) from E. coli K-12 substr. MG1655 (αcc. no. NP_418185, NP_417643, NP_418186, respectively) were codon-optimized and obtained by gene synthesis. The operon glmUM was cloned under the control of constitutive tetracyclin promoter Pₜₑₜ while glmS was cloned under the constitutive P_{T5} promoter. The transposon cassette <Pₜₑₜ-glmUM-P_{T5}-gImS-FRT-dhfr-FRT> (SeqID3), flanked by the inverted terminal repeats specifically recognized by the mariner-like element Himar1 transposase was inserted from pEcomar-glmUM-glmS revealing a lacto-N-triose II production strain. Additionally, the expression fragment <Pₜₑₜ-lacY (6HIS)-FRT-aadA-FRT> (SeqID4) was integrated by using the EZ-Tn5 transposase.

The gal-operon (galETKM) was amplified from E. coli K12 TG1 (SeqID6) using primer 605 and 606 and inserted into the galM ybhJ locus of E. coli BL21 (DE3) strain by homologous recombination facilitated by using the red recombinase helper plasmid pKD46 (Datsenko and Warner, Proc. Natl. Acad. Sci. 2000, USA 97:6640-6645). Sequences of the heterologous genes and gene clusters are deposit in appendix 1.

### Example 2

Batch Fermentation of E. coli BL21 (DE3) 707 Screening Various β-1,3-N-acetyl-glycosaminyltransferases

The gene for the B-1,3-N-acetyl-glucosaminyltransferase Pmnag T from Pasteurella multocida subsp. multocida str. HN06 (αcc. no. PMCN06_0022) was codon-optimized and synthetically synthesized by GenScript Cooperation (Piscataway, USA). Cloning of the gene occurred by sequence and ligation-independent cloning into the plasmid pET-DUET (Merck KGaA, Darmstadt, Germany). All primer used for cloning are listed in table 3 below.

E. coli BL21 (DE3) 707 (table 2 below) harbouring plamid pET-PmnagT coding for a 3-1,3-N-acetyl glucosaminyltransferase was grown at 30° C. in mineral salts medium (Samain et al., J. Biotech. 1999, 72:33-47) supplemented with 2% (wt/vol) glucose and ampicillin 100 μg ml⁻¹. When the cultures reached an OD660nm of 0.1, gene expression was induced by addition of 0.3 mM IPTG. After four hours of incubation 1.5 mM lactose was added. After an additional incubation for 24 hours at 30° C. in shaking flasks cells were harvested. LNT-2 was detected by thin layer chromatography. Therefore, cells were mechanically disrupted in a defined volume using glass beads. Subsequently, samples were applied on TLC Silica Gel 60 F₂₅₄ (Merck KGaA, Darmstadt, Germany). The mobile phase was composed of acetone: butanol: acetic acid: water (35:35:7:23).

The result of the TLC analysis is shown in FIG. 2. The formation of a compound showing the same migration rate as the trisaccharide standard LNT-II could be observed when the gene PmnagT was overexpressed. The LNT-II production strain 724 served as a control (19). Standards for lactose (1) and LNT-II (2) are depicted. LNT-II product formation in the samples is marked by asterisks.

### Example 3

Generation of an E. coli Lacto-N-triose II Production Strain Overexpressing a Homologous Sugar Efflux Transporter

The export of oligosaccharides produced in E. coli was proven to be a limiting factor during the fermentation process. However, trisaccharides like 2′-fucosyllactose and LNT-2 are translocated into the culture supernatant to some extent, thus probably encoding a working sugar efflux transporter. In order to improve the efflux of lacto-N-triose II (LNT-II; GluNAc (β1-3) Gal (β1-4) Glc), the E. coli BL21 (DE3) strain 1326 (table 2 below) was used for the screening of a library of sugar efflux transporters (SET). Putative SET proteins from E. coli were amplified from genomic DNA of E. coli BL21 (DE3) and integrated into vector pINT by sequence and ligation-independent cloning. Using the example of the gene yjhB, the primer 2567, 2568, 2526 and 2443 were used, generating the plasmid pINT-yjhB. The primer sequences used for cloning are listed in table 3 below.

E. coli BL21 (DE3) 1326 harbouring plamids encoding for 20 different E. coli transporters were grown at 30° C. in mineral salts medium (Samain et al., J. Biotech. 1999, 72:33-47) supplemented with 2% (wt/vol) glucose, ampicillin 100 μg ml⁻¹ and zeocin 40 μg ml⁻¹. When the cultures reached an OD660nm of 0.1, gene expression of the genes was induced by addition of 200 ng/ml anhydrotetracycline. After four hours of incubation 2.5 mM lactose was added. After an additional incubation for 24 and 48 hours at 30° C. in shaking flasks the LNT-II concentration in the supernatant was determined by LC-MS.

Mass analysis was performed by characteristic fragment ion detection using an LC Triple-Quadrupole MS detection system. Precursor ions are selected and analyzed in quadrupole 1, fragmentation takes place in the collision cell using nitrogen as CID gas, selection of fragment ions is performed in quadrupole 3.

Lacto-N-tetraose (LNT (Gal (β1-3) GlcNAc (β1-3) Gal (β1-4) Glc)), LNT-II and Maltotriose (internal standard for quantification) were analyzed in ESI positive ionization mode. LNT forms an ion of m/z 708.3 [M+H⁺], LNT-II an ion of m/z 546.1 [M+H⁺] and Maltotriose an ion of m/z 522.0 [M+NH₄⁺]. Adduct formation of this carbohydrate [m/z 504.0] takes place with an ammonium ion (NH4⁺), resulting in mass shift of +18. Thus for Maltotriose a precursor ion of m/z 522.0 was selected. The precursor ion was further fragmented in the collision cell into the characteristic fragment ions m/z 487.1, m/z 325.0 and m/z 163.2. The molecular ion of LNT (m/z 708.3) was fragmented into m/z 546.3, m/z 528.3, m/z 366.2 and m/z 204.0. LNT-II (m/z 546.1) was fragmented into m/z 204.2, 186.0, 138.0 and 126.0 (see method description).

Chromatographic separation of LNT and LNT-II was performed on a Luna NH2 HPLC column (Phenomenex, Aschaffenburg, Germany). This was necessary due to partial fragmentation of LNT during ionization resulting in LNT-II signals affecting quantification results of the individual carbohydrates.

Only for the strain expressing the gene yjhB, an increased amount of LNT-2 in the culture supernatant was observed (see table 1 below).

TABLE 1
Calculated concentrations of LNT-II in the culture
supernatant of an E. coli BL21 (DE3) strain
overexpressing yjhB and the reference strain.

| | Calc. conc. | Calc. conc. | |
|---|---|---|---|
| | after 24 h of | after 48 h of | |
| | incubation | incubation | Analyte |
| Sample | [μM] | [μM] | RT |
| 1326 | 751 | 1265 | 0.616 |
| 1326 pINT-yjhB | 413 | 1975 | 0.609 |

### Example 4

Batch fermentations of E. coli BL21 (DE3) 724 Screening Various β-1,4-galactosyltransferases

The genes for the-1,4-galactosyltransferases lex1 from Aggregatibacter aphrophilus NJ8700 (acc. no. YP_003008647), PmgalT7 from Pasteurella multocida subsp. multocida str. HN06 (acc. No. PMCN06_0021), MsgalT8 from Myxococcus stipitatus DSM14675 (acc. no. MYSTI_04346), KdgalT10 from Kingella denitrificans ATCC 33394 (acc. no. HMPREF9098_2407), gatD from Pasteurella multocida M1404 (acc. no. GQ444331), BfgalT2 from Bacterioidis fragilis NCTC9343 (acc. no. BF9343_0585), IsgD from Haemophilus influenza (acc. no. AAA24981) and HpgalT from Helicobacter pylori (acc. no. AB035971) were codon-optimized and synthetically synthesized by GenScript Cooperation (Piscataway, USA). Cloning of the genes occurred by sequence and ligation-independent cloning (Li and Elledge, Nat Methods. 2007 March;4 (3): 251-6.). Therefore, the plasmid pINT, harbouring the malE gene under control of an anhydrotetracyline-inducible promoter, was used, enabling the generation of a N-terminal fusion of the β-1,4-galactosyltransferase genes with malE. Solely, the β-1,4-galactosyltransferase encoding gene waaX from Pectobacterium atrosepticum JG10-08 (acc. no. ECA0154) was cloned into plasmid pACYC-Duet (Merck KGaA, Darmstadt, Germany). All primer used for cloning are listed in table 3 below.

E. coli BL21 (DE3) 724 (table 2 below) harbouring plamid pCDF-galE and a plasmid coding for the gene fusion of malE with a β-1,4-galactosyltransferase was grown at 30° C. in mineral salts medium (Samain et al., J. Biotech. 1999, 72:33-47) supplemented with 2% (wt/vol) glucose, ampicilin 100 μg ml⁻¹ and zeocin 40 μg ml⁻¹. When the cultures reached an OD660nm of 0.1, gene expression of the galE gene and the β-1,4-galactosyltransferase was induced by addition of 0.3 mM IPTG and 200 ng/ml anhydrotetracycline. E. coli BL21 (DE3) 534 (table 2 below) harbouring plamids pET-IgtA, pCOLA-glmUM-glmS, pCDF-galT-galE and pACYC-waaX was grown at 30° C. in mineral salts medium supplemented with 2% (wt/vol) glucose, ampicillin 100 μg ml⁻¹, chloramphenicol 34 μg ml⁻¹, streptomycin 50 μg ml⁻¹ and kanamycin 30 μg ml⁻¹. When the cultures reached an OD660nm of 0.1, gene expression was induced by addition of 0.3 mM IPTG. Four hours after induction of gene expression 2 mM lactose were added. After an additional incubation for 48 hours at 30° C. in shaking flasks, cells were harvested and mechanically disrupted. Lacto-N-neotetraose (LNnT (Gal (β1-4) GlcNAc (β1-3) Gal (β1-4) Glc)) was detected by thin layer chromatography. Therefore, cells were mechanically disrupted using glass beads. Subsequently, samples were applied on TLC Silica Gel 60 F254 (Merck KGaA, Darmstadt, Germany). The mobile phase was composed of acetone: butanol: acetic acid: water (35:35:7:23).

The results of the TLC analyses are shown in FIGS. 3-5. FIG. 3 shows the TLC analysis of culture extracts of lacto-N-tetraose (LNT) producing E. coli BL21 (DE3) strains overexpressing the β-1,4-galactosyltransferase encoding genes BfgalT2 (1), PmgalT7 (3), MsgalT8 (6), gatD (7), lex1 (9), lgtB (11) or IsgD (13). Standards for lactose (15), LNT-II (16) and LNnT (17) are depicted. LNnT product formation in the samples is marked by asterisks.

FIG. 4 shows the TLC analysis of culture extracts of lacto-N-tetraose (LNT) producing E. coli BL21 (DE3) strains overexpressing the β-1,4-galactosyltransferase encoding genes KdgalT10 (1), cpsl14J (7), cpslaJ (8, 9), HpgalT (12). Standards for lactose (3, 15), LNT-II (4, 16) and LNnT (5, 17) are depicted. LNnT product formation in the samples is marked by asterisks.

FIG. 5 shows the TLC analysis of culture extracts of lacto-N-tetraose producing E. coli BL21 (DE3) strains overexpressing the β-1,4-galactosyltransferase encoding gene waaX (5). Standards for lactose (1), LNT-II and LNnT (2) are depicted. Again, LNnT product formation in the samples is marked by asterisks.

The formation of a compound showing the same migration rate as the tetrasaccharide standard LNnT could be observed when the following genes were overexpressed: lex1, PmgalT7, MsgalT8, BfgalT2, gatD, IsgD, KdgalT10, HpgalT, wax.

The 3-1,4-galactosyltransferases cpslaJ and cpsl14J, known from literature to produce LNnT (Watanabe et al., J Biochem. 2002 February;131 (2): 183-91; Kolkman et al., J Bacteriol. 1996 July;178 (13): 3736-41), were also included in the activity screening and served as positive control. Using the described expression system, the formation of LNnT could be observed by CpslaJ and Cpsl14J (FIG. 3). In total, 11 out of 30 tested genes were observed to produce LNnT from LNT-Il and UDP-galactose.

### Example 5

Batch fermentations of E. coli BL21 (DE3) 534 screening different β-1,3-galactosyltransferases

Using genomic DNA of E. coli K12 DH5a as template, galE was amplified using primer 1163 and 1162. The PCR product was purified, restricted with restriction endonucleases Ndel and Xhol and ligated into the second multiple cloning site of vector pCDFDuet (Merck KGaA, Darmstadt, Germany), which was cut with the same enzymes. GalE is expressed from the IPTG inducible T7 promoter. The E. coli K12 gene galT was amplified from genomic DNA and integrated into plasmid pCDF-galE by sequence and ligation-independent cloning using primer 991-994, producing the plasmid pCDF-galT-galE.

Using the codon-optimized gene of IgtA as template, amplification occurred using primer 688 and 689. The PCR product was purified, restricted with restriction endonucleases Ndel and Aatll and ligated into the multiple cloning site of vector pETDuet (Merck KGaA, Darmstadt, Germany), which was cut with the same enzymes, producing the plasmid pET-IgtA.

Cloning of the codon-optimized gene construct of glmUM occurred by sequence and ligation-independent cloning into the plasmid pCOLA-Duet (Merck KGaA, Darmstadt, Germany) using primer 848-851. The codon-optimized form of glmS was amplified using primer 852 and 853. The PCR product was purified, restricted with restriction endonucleases Ndel and Aatll and ligated into the second multiple cloning site of vector pCOLA-glmUM, which was cut with the same enzymes, producing the plasmid pCOLA-gimUM-glmS.

The genes for the β-1,3-galactosyltransferases wbdO from Salmonella enterica subsp. salamae serovar Greenside (acc. no. AY730594) and furA from Lutiella nitroferrum 2002 (FuraDRAFT_0419) were also codon-optimized and synthetically synthesized by GenScript Cooperation (Piscataway, USA). Cloning of the genes occurred by sequence and ligation-independent cloning into the plasmid pACYC-Duet (Merck KGaA, Darmstadt, Germany). All primer used for cloning are listed in table 3 below.

E. coli BL21 (DE3) 534 harbouring plasmids pET-IgtA, pCOLA-glmUM-glmS, pCDF-galT-galE and a plasmid coding for a B-1,3-galactosyltransferase pACYC-furA or pACYC-wbdO was grown at 30° C. in mineral salts medium (Samain et al., J. Biotech. 1999, 72:33-47) supplemented with 2% (w/v) glucose, ampicillin 100 μg ml⁻¹, chloramphenicol 34 μg ml⁻¹, streptomycin 50 μg ml⁻¹ and kanamycin 30 μg ml⁻¹. When the cultures reached an OD660nm of 0.1, gene expression was induced by addition of 0.3 mM IPTG. After four hours of incubation 2 mM lactose was added. After an additional incubation for 48 hours at 30° C. in shaking flasks, cells were harvested. LNT was detected by thin layer chromatography. Therefore, cells were mechanically disrupted using glass beads. Subsequently, samples were applied on TLC Silica Gel 60 F254 (Merck KGaA, Darmstadt, Germany). The mobile phase was composed of acetone: butanol: acetic acid: water (35:35:7:23).

The results of the TLC analyses are shown in FIG. 6, showing TLC analysis of culture extracts of lacto-N-tetraose producing E. coli BL21 (DE3) strains overexpressing the β-1,3-galactosyltransferase encoding genes wbdO or furA. LNT product formation in the samples is marked. Out of 12 tested putative β-1,3-galactosyltransferases, the formation of a compound showing the same migration rate as the tetrasaccharide standard LNT could only be observed when genes wbdO and furA were overexpressed.

### Example 6

Development of an Improved Plasmid-free E. coli Lacto-N-tetraose Production Strain

Escherichia coli BL21 (DE3) strain 724 was used to construct a lacto-N-tetraose (LNT) producing strain. Metabolic engineering included the genomic integration of the transposon cassettes <Pₜₑₜ-wbdO-P_{T5}-galE-FRT-cat-FRT> (SeqID5), flanked by the inverted terminal repeats specifically recognized by the mariner-like element Himar1 transposase, which was inserted from pEcomar-wbdO-galE. The resulting strain 1353 was further metabolically engineered to exhibit an increased intracellular LNT-II pool resulting in the elevated production of LNT. Therefore, the mayor facilitator superfamily transporter yjhB (acc. no. YP_003001824) was deleted from the genome of the E. coli strain, generating strain 1431 (table 2 below).

Batch fermentation of the E. coli BL21 (DE3) strains 1353 and 1431 was conducted for 48 hours at 30° C. in mineral salts medium (Samain et al., J. Biotech. 1999, 72:33-47) containing 2% (wt/vol) glucose as sole carbon and energy source. When the cultures reached an OD660nm of 0.5, 2.5 mM lactose was added. The presence of LNT-II and LNT in the culture supernatant was detected by high performance liquid chromatography (HPLC).

Analysis by HPLC was performed using a refractive index detector (RID-10A) (Shimadzu, Duisburg, Germany) and a ReproSil Carbohydrate, 5 μm (250 mm×4,6 mm) (Dr. Maisch GmbH, Germany) connected to an HPLC system (Shimadzu, Duisburg, Germany). Elution was performed isocratically with acetonitril: H₂O (68/32 (v/v)) as eluent at 35° C. and a flow rate of 1.4 ml/min. 40 μl of the sample were applied to the column. Samples were filtered (0,22 μm pore size) and cleared by solid phase extraction on an ion exchange matrix (Strata ABW, Phenomenex, Aschaffenburg, Germany).

The results of the HPLC analyses are shown in FIG. 7, showing HPLC analyses of the culture supernatant of lacto-N-tetraose producing E. coli BL21 (DE3) strain. (A) Supernatant of E. coli BL21 (DE3) 1353 (black graph) and 1431 (pink graph) grown in the presence of glucose and lactose after 24 h of incubation. (β) Supernatant of E. coli BL21 (DE3) 1353 (blue graph) and 1431 (brown graph) grown in the presence of glucose and lactose after 48 h of incubation. As can be seen from the HPLC analyses, the deletion of yjhB in a LNT producing strain resulted in an elevated accumulation of LNT in the culture supernatant.

### Example 7

Generation of an E. coli Lacto-N-tetraose Production Strain Overexpressing a Sugar Efflux Transporter

Since an export of lacto-N-tetraose into the medium is only moderate for production strains, a screening of a sugar efflux transporter library was conducted. In accordance to example 3 putative SET proteins were either amplified from E. coli genomic DNA or were codon-optimized and synthetically synthesized by GenScript Cooperation (Piscataway, USA). Following amplification genes were integrated into vector pINT by sequence and ligation-independent cloning. The primer design for the cloning of E. coli genes was in accordance to example 3. Synthetic genes were synthesized with standardized nucleotide overhangs and likewise integrated into the expression vector using the primer 2527, 2444, 2526 and 2443. The primer sequences used for cloning are listed in table 3 below.

E. coli BL21 (DE3) 1353 (table 2 below) harbouring plasmids encoding for 66 different transporters were grown at 30°° C. in mineral salts medium (Samain et al., J. Biotech. 1999, 72:33-47) supplemented with 3% (w/v) glucose, 5 g l⁻¹ NH₄Cl₂, ampicillin 100 μg ml⁻¹ and kanamycin 15 μg ml⁻¹. Precultivation appeared in 96-well plates harbouring a total volume of 200 μl. After 24 h of incubation at 30° C. by continuous shaking, 50 μl per well was transferred into 96-well deep well plates harbouring a total volume of 400 μl mineral salts medium additionally supplemented with 200 ng ml⁻¹ anhydrotetracycline and 10 mM lactose. After a sustained incubation for 24 to 48 hours the LNT concentrations in the supernatant were determined by LC-MS. Mass analysis was performed as described in example 3.

FIG. 8 shows the relative concentration of lacto-N-tetraose in the supernatant of E. coli BL21 (DE3) strains overexpressing sugar efflux transporters compared to the control strain 1353. The LNT titer of strain 1353 was set to 100%. As shown in FIG. 8, the overexpression of 11 out of 66 genes resulted in a doubled LNT production. Among these, also a protein encoded in the genome of E. coli BL21 (DE3) proved to enhance the LNT export (TP37, yebQ, acc. no. NC_012971). YebQ is a predicted MFS transporter, putatively involved in multi drug efflux, which might represent a responsible transporter protein that realizes the observed basal efflux of LNT during fermentation of strain 1353.

Furthermore, the exporters encoded by the genes spoVB of Bacillus amyloliquefaciens (TP1, acc. no. AFJ60154), yabM of Erwinia pyrilfoliae (TP2, acc. no. CAY73138), bcr of E. coli MG1655 (TP18, acc. no. AAC75243), ydeA of E. coli MG1655 (TP20, acc. no. AAC74601), proP2 of Haemophilus parainfluenzae (TP54, acc. no. EGC72107), setA of Pectobacterium carotovorum (TP55, acc. no. ZP_03829909), fucP of E. coli MG1655 (TP59, acc. no. AIZ90162), mdeA of Staphylococcus aureus Bmb9393 (TP61, acc. no. SABB_01261), ImrA of Lactococcus lactis (TP62, acc. no. L116532), setA of Pseudomonas sp. MT-1 (TP72, acc. no. BAP78849) and setA of Beauveria bassiana D1-5 (TP73, acc. no. KGQ13398) resulted in an increased LNT production when overexpressed in the E. coli production strain 1353.

### Example 8

Generation of an E. coli Lacto-N-triose II Production Strain by Overexpression of Heterologous Sugar Efflux Transporters

The LNT exporter screening described in example 6 interestingly disclosed two proteins-TP11 from Mannheimia succiniciproducens MBEL55E (proP, acc. no. AAU37785) and TP70 from Cedecea neteri M006 (setA, acc. no. WP_039290253)-whose overexpression resulted in a significantly increased production of LNT-II and consequently in a decreased LNT production (data not shown). This observation was confirmed in an experimental setup as described in example 3. The overexpression of the sugar efflux transporter YjhB served as a positive control. The overexpression of TP11 as well as TP70 resulted in an approximately 4-fold increase in LNT-II production which was even slightly more than for YjhB: FIG. 9 shows a diagram displaying the concentrations of lacto-N-triose II in the supernatant of E. coli BL21 (DE3) strains overexpressing the sugar efflux transporters TP11 (2), YjhB (3) or TP70 (4). Strain 1326 harbouring an empty control plasmid served as a control (1). Thus, 3 sugar efflux transporters were identified which target LNT-II for export and whose overexpression might be useful to engineer a LNT-II production strain.

TABLE 2
Strains and plasmids

| Strain | Genotype | Ref. |
|---|---|---|
| E. coli BL21(DE3) | F-ompT hsdSB(rB-, mB-) gal dcm (DE3) | Merck KGaA, |
| | | Darmstadt, |
| | | Germany |
| E. coli BL21(DE3) 534 | E. coli BL21(DE3) ΔlacZ Δara ΔwcaJ | This study |
| | ΔfucIK ΔnagAB harbouring genomic | |
| | integrations of: galETKM, lacy | |
| E. coli BL21(DE3) 724 | E. coli BL21(DE3) ΔlacZ Δara ΔwcaJ | This study |
| | ΔfucIK ΔnagAB harbouring genomic | |
| | integrations of: galETKM, lacY, IgtA-galT- | |
| | kanR, glmUM-glmS-dhfr | |
| E. coli BL21(DE3) 1326 | E. coli BL21(DE3) ΔlacZ Δara ΔwcaJ | This study |
| | ΔfucIK ΔnagAB harbouring genomic | |
| | integrations of: galETKM, lacY, IgtA-galT- | |
| | kanR, glmUM-glmS-dhfr, lacy(6HIS)-aadA | |
| E. coli BL21(DE3) 707 | E. coli BL21(DE3) ΔlacZ Δara ΔwcaJ | This study |
| | ΔfucIK ΔnagAB harbouring genomic | |
| | integrations of: galETKM, lacY, glmUM- | |
| | glmS-dhfr | |
| E. coli BL21(DE3) 1353 | E. coli BL21(DE3) ΔlacZ Δara ΔwcaJ | This study |
| | ΔfucIK ΔnagAB harbouring genomic | |
| | integrations of: galETKM, lacY, IgtA-galT- | |
| | kanR, glmUM-glmS-dhfr, wbdO-galE-cat | |
| E. coli BL21(DE3) 1431 | E. coli BL21(DE3) ΔlacZ Δara ΔwcaJ | This study |
| | ΔfucIK ΔnagAB harbouring genomic | |
| | integrations of: galETKM, lacY, IgtA-galT- | |
| | kanR, glmUM-glmS-dhfr, wbdO-galE-cat, | |
| | ΔyjhB-aacC1 | |
| pCDF-galE | galE of E. coli K12 integrated into vector | EP 14 162 869.3 |
| | pCDFDuet | |
| pET-IgtA (SeqID7) | IgtA of Neisseria meningitidis integrated | This study |
| | into vector pETDuet | |
| pCDF-galT-galE (SeqID8) | galT and galE of Escherichia coli K12 | This study |
| | integrated into vector pCDFDuet | |
| pCOLA-glmUM-glmS | glmU, glmM and glmS of Escherichia coli | This study |
| (SeqID9) | K12 integrated into vector pCOLADuet | |
| pINT-malE-lex1 | Gene fusion of malE with lex-1 of | EP 14 162 869.3 |
| | Aggregatibacter aphrophilus NJ8700 | |
| | integrated into vector pINT | |
| pINT-malE-PmgalT7 | Gene fusion of PmgalT7 of Pasteurella | This study |
| (SeqID10) | multocida subsp. multocida str. HN06 | |
| | integrated into vector pINT | |
| pINT-malE-MsgalT8 | Gene fusion of MsgalT8 of Myxococcus | This study |
| | stipitatus DSM14675 integrated into vector | |
| SeqID11) | pINT | |
| pINT-malE-KdgalT10 | Gene fusion of KdgalT10 of Kingella | This study |
| (SeqID12) | denitrificans ATCC 33394 integrated into | |
| | vector pINT | |
| pINT-malE-gatD (SeqID13) | Gene fusion of gatD of Pasteurella | This study |
| | multocida M1404 integrated into | |
| | vector pINT | |
| pINT-malE-BFgalT2 | Gene fusion of BfgalT2 of Bacterioidis | This study |
| (SeqID14) | fragilis NCTC9343 integrated into | |
| | vector pINT | |
| pINT-malE-IsgD (SeqID15) | Gene fusion of IsgD of Haemophilus | This study |
| | influenza integrated into vector pINT | |
| pINT-malE-HPgalT | Gene fusion of HpgalT of Helicobacter | This study |
| (SeqID16) | pylori integrated into vector pINT | |
| pACYC-waaX (SeqID17) | waaX of Pectobacterium atrosepticum | This study |
| | JG10-08 integrated into vector pACYCDuet | |
| pACYC-wbdO (SeqID18) | wbdO of Salmonella enterica subsp. | This study |
| | salamae serovar Greenside integrated into | |
| | vector pACYCDuet | |
| pACYC-furA (SeqID19) | furA of Lutiella nitroferrum 2002 integrated | This study |
| | into vector pACYCDuet | |
| pET-PmnagT (SeqID20) | PmnagT of Pasteurella multocida subsp. | This study |
| | multocida str. HN06 integrated into vector | |
| | pETDuet | |
| pINT-yjhB (SeqID21) | yjhB of E. coli BL21 DE3 integrated into | This study |
| | vector pINT | |
| pINT-yebQ (SeqID22) | yebQ of E. coli BL21 DE3 integrated into | This study |
| | vector pINT | |
| pINT-proP (SeqID23) | proP of Mannheimia succiniciproducens | This study |
| | MBEL55E integrated into vector pINT | |
| pINT-Cn-setA (SeqID24) | setA of Cedecea neteri M006 integrated | This study |
| | into vector pINT | |
| pINT-spoVB (SeqID25) | spoVB of Bacillus amyloliquefaciens | This study |
| | integrated into vector pINT | |
| pINT-yabM (SeqID26) | yabM of Erwinia pyrifoliae integrated into | This study |
| | vector pINT | |
| pINT-ydeA (SeqID27) | ydeA of E. coli MG1655 integrated into | This study |
| | vector pINT | |
| pINT-proP2 (SeqID28) | proP2 of Haemophilus parainfluenzae | This study |
| | integrated into vector pINT | |
| pINT-Pc-setA (SeqID29) | setA of Pectobacterium carotovorum | This study |
| | integrated into vector pINT | |
| pINT-fucP (SeqID30) | fucP of Escherichia coli BL21 (DE3) | This study |
| | integrated into vector pINT | |
| pINT-mdeA (SeqID31) | mdeA of Staphylococcus aureus Bmb9393 | This study |
| | integrated into vector pINT | |
| pINT-ImrA (SeqID32) | ImrA of Lactococcus lactis integrated into | This study |
| | vector pINT | |
| pINT-Ps-setA (SeqID33) | setA of Pseudomonas sp. MT-1 integrated | This study |
| | into vector pINT | |
| pINT-Bb-setA (SeqID34) | setA of Beauveria bassiana D1-5 | This study |
| | integrated into vector pINT | |

TABLE 3
Oligonucleotides used for PCR

| Primer | Sequence 5′-3′ |
|---|---|
| 605 KI gal fwd | TTACTCAGCAATAAACTGATATTCCGTCAGGCTGG (SeqID35) |
| 606 KI gal rev | TTGTAATCTCGCGCTCTTCACATCAGACTTTCCATATAGAGCGTAATTTC |
| | CGTTAACGTCGGTAGTGCTGACCTTGCCGGAGG (SeqID36) |
| 1119 ME-for | CTGTCTCTTATCACATCTCCTGAAATGGCCAGATGTAATTCCTAATTTTT |
| | GTTG (SeqID37) |
| 1120 ME rev | CTGTCTCTTATCACATCTCACATTACATCTGAGCGATTGTTAGG |
| | (SeqID38) |
| 1163 galE_NdeI-for | GATCACATATGAGAGTTCTGGTTACCGGTG (SeqID39) |
| 1164 galE_XhoI-rev | GATCACTCGAGTCATTAATCGGGATATCCCTGTGGATGGC (SeqID40) |
| 5176 lex1 pINT-f | GTCGATGAAGCCCTGAAAGACGCGCAGACTATGCACTTCATTGAAAAC |
| | AAAAACTTCGTC (SeqID41) |
| 5177 lex1 pINT-r | GATGGCCTTTTTGCGTGTCGACGCGGCCGCCTAGATAAACAGGATGAT |
| | ATTTTTGCCTIG (SeqID42) |
| 5178 pINT lexl-f | CAAGGCAAAAATATCATCCTGTTTATCTAGGCGGCCGCGTCGACACGC |
| | AAAAAGGCCATC (SeqID43) |
| 5179 pINT lexl-r | GACGAAGTTTTTGTTTTCAATGAAGTGCATAGTCTGCGCGTCTTTCAGG |
| | GCTTCATCGAC (SeqID44) |
| 5192 waaX pINT for | GTCGATGAAGCCCTGAAAGACGCGCAGACTATGATTGATAACCTGATTA |
| | AGCGTACCCCG (SeqID45) |
| 5193 waaX pINT rev | ATGGCCTTTTTGCGTGTCGACGCGGCCGCTTAATTCGAGCGGGTAAAG |
| | ATCTTCATCAGG (SeqID46) |
| 5194 pINT waaX for | CTGATGAAGATCTTTACCCGCTCGAATTAAGCGGCCGCGTCGACACGC |
| | AAAAAGGCCATC (SeqID47) |
| 5195 pINT waaX rev | CGGGGTACGCTTAATCAGGTTATCAATCATAGTCTGCGCGTCTTTCAGG |
| | GCTTCATCGAC (SeqID48) |
| 5164 PmgalT7 pINT | GTCGATGAAGCCCTGAAAGACGCGCAGACTATGAGCGGTGAACACTAT |
| for | GTCATTAGCCTG (SeqID49) |
| 5165 PmgalT7 pINT | GATGGCCTTTTTGCGTGTCGACGCGGCCGCTCATTTAAATTCGATGATC |
| rev | ATCTTGTCGTT (SeqID50) |
| 5166 pINT PmgalT7 | AACGACAAGATGATCATCGAATTTAAATGAGCGGCCGCGTCGACACGC |
| for | AAAAAGGCCATC (SeqID51) |
| 5167 pINT PmgalT7 | CAGGCTAATGACATAGTGTTCACCGCTCATAGTCTGCGCGTCTTTCAGG |
| rev | GCTTCATCGAC (SeqID52) |
| 5168 MsgalT8 pINT | GTCGATGAAGCCCTGAAAGACGCGCAGACTATGGATGAAATCAAACTG |
| for | TCGGTGGTTATG (SeqID53) |
| 5169 MsgalT8 pINT | GATGGCCTTTTTGCGTGTCGACGCGGCCGCTCATTGGCGACGCCAATC |
| rev | GAACGCAACGCG (SeqID54) |
| 5170 pINT MsgalT8 | CGCGTTGCGTTCGATTGGCGTCGCCAATGAGCGGCCGCGTCGACACG |
| for | CAAAAAGGCCATC (SeqID55) |
| 5171 pINT MsgalT8 | CATAACCACCGACAGTTTGATTTCATCCATAGTCTGCGCGTCTTTCAGG |
| rev | GCTTCATCGAC (SeqID56) |
| 5561 KdgalT10 | GTCGATGAAGCCCTGAAAGACGCGCAGACTATGGAAAACTATGTCGTC |
| pINT for | TCTATCCGCACC (SeqID57) |
| 5562 KdgalT10 | GATGGCCTTTTTGCGTGTCGACGCGGCCGCTCATTTGAACGGAACAAT |
| pINT-rev | CTTTTTGTCATC (SeqID58) |
| 5563 pINT- | GATGACAAAAAGATTGTTCCGTTCAAATGAGCGGCCGCGTCGACACGC |
| KdgalT10 for | AAAAAGGCCATC (SeqID59) |
| 5564 pINT- | GGTGCGGATAGAGACGACATAGTTTTCCATAGTCTGCGCGTCTTTCAG |
| KdgalT10 rev | GGCTTCATCGAC (SeqID60) |
| 5172 gatD pINT for | GTCGATGAAGCCCTGAAAGACGCGCAGACTATGTCCTCAGCTTTCCATT |
| | ACGTCATTAGC (SeqID61) |
| 5173 gatD pINT rev | GATGGCCTTTTTGCGTGTCGACGCGGCCGCTCATTCAAATTCGATAATC |
| | ATGGTGATTTT (SeqID62) |
| 5174 pINT gatD for | AAAATCACCATGATTATCGAATTTGAATGAGCGGCCGCGTCGACACGCA |
| | AAAAGGCCATC (SeqID63) |
| 5175 pINT gatD rev | GCTAATGACGTAATGGAAAGCTGAGGACATAGTCTGCGCGTCTTTCAG |
| | GGCTTCATCGAC (SeqID64) |
| 5160 BfglaT2 pINT | GTCGATGAAGCCCTGAAAGACGCGCAGACTATGAACGTGAATAAGCCG |
| for | ACCACCGAAAAG (SeqID65) |
| 5161 BfgalT2 pINT | GATGGCCTTTTTGCGTGTCGACGCGGCCGCTCAGTATTCTTCAATTTTG |
| rev | TCCAGTTGATA (SeqID66) |
| 5162 pINT BfgalT2 | TATCAACTGGACAAAATTGAAGAATACTGAGCGGCCGCGTCGACACGC |
| for | AAAAAGGCCATC (SeqID67) |
| 5163 pINT BfgalT2 | CTTTTCGGTGGTCGGCTTATTCACGTTCATAGTCTGCGCGTCTTTCAGG |
| rev | GCTTCATCGAC (SeqID68) |
| 5746 | GTGATCAACGCCGCCAGCGGTCGTCAGACTGTCGATGAAGCCCTGAAA |
| | GACGCGCAGACT (SeqID69) |
| 5747 | GCGGCCGCGTCGACACGCAAAAAGGCCATCCATCCGTCAGGATGGCC |
| | TTCTGCTTAATTT (SeqID70) |
| 5748 | AAATTAAGCAGAAGGCCATCCTGACGGATGGATGGCCTTTTTGCGTGT |
| | CGACGCGGCCGC (SeqID71) |
| 5749 | AGTCTGCGCGTCTTTCAGGGCTTCATCGACAGTCTGACGACCGCTGGC |
| | GGCGTTGATCAC (SeqID72) |
| 1886 SLIC wbdO | GTTTAACTTTAATAAGGAGATATACCATGCTGACGGAAGTGCGCCCGGT |
| pACYC for | CTCTACGACGAAACCGC (SeqID73) |
| 1887 SLIC wbdO | CGACCTGCAGGCGCGCCGAGCTCGAATTCATTTGATGTATTTGCAATA |
| pACYC rev | GAACACAGAAAAGACCGT (SeqID74) |
| 1888 SLIC pACYC | GTGTTCTATTGCAAATACATCAAATGAATTCGAGCTCGGCGCGCCTGCA |
| wbdo rev | GGTCGACAAGCTTGCGG (SeqID75) |
| 1889 SLIC pACYC | GAGACCGGGCGCACTTCCGTCAGCATGGTATATCTCCTTATTAAAGTTA |
| Wbd0 For | AACAAAATTATTTCTACAGG (SeqID76) |
| 1890 SLIC pACYC | GTATGGTGACCCTGTGGCGCAAATGAGAATTCGAGCTCGGCGCGCCTG |
| furA rev | CAGGTCGACAAGCT (SeqID77) |
| 1891 SLIC pACYC | GCGCTGCCCTGTTTGATTTTATCCATGGTATATCTCCTTATTAAAGTTAA |
| furA for | ACAAAATTATTTCT (SeqID78) |
| 1892 SLIC furA | CCTGCAGGCGCGCCGAGCTCGAATTCTCATTTGCGCCACAGGGTCACC |
| pACYC rev | ATACGTGCCGGCAGG (SeqID79) |
| 1893 SLIC furA | GITTAACTTTAATAAGGAGATATACCATGGATAAAATCAAACAGGGCAG |
| pACYC for | CGCCTCTCTGGTTGTCG (SeqID80) |
| 3055 SLIC PmnagT | CAGACTCGAGGGTACCGACGTCCTAATAAGTAGATGAATATTTATCAGG |
| pET rev | ACGAAGAT (SeqID81) |
| 3056 SLIC pET | AACTAAAGGTTTATTTTCCATATGTATATCTCCTTCTTATACTTAACTAAT |
| PmnagT for | ATAC (SeqID82) |
| 3057 SLIC pET | TAAATATTCATCTACTTATTAGGACGTCGGTACCCTCGAGTCTGGTAAA |
| PmnagT rev | GAAACCGCTGCTGCG (SeqID83) |
| 3058 SLIC PmnagT | GTATAAGAAGGAGATATACATATGGAAAATAAACCTTTAGTTTCAGTTTT |
| pET for | GATTTGTGC (SeqID84) |
| 2567_SLIC_yjhB-for | TAACTTTAAGAAGGAGATATACAAGAGCTCGAGTCGAAGGAGATAGAAC |
| | CATGGCAACAGCATGGTATAAACAAG (SeqID85) |
| 2568_SLIC_yjhB- | GCGTGTCGACGCGTTTAGAGGCCCCAAGGGGTTATGCTAGTATCGATT |
| rev | TATCATTTAGCCACGGATAGTTTATAAATTTTAC (SeqID86) |
| 2526_SLIC_pINT_ | GGTTCTATCTCCTTCGACTCGAGCTCTTGTATATCTCCTTCTTAAAGTTA |
| TP-rev | AACAAAATTATTTCTAGATTTTTGTCGAAC (SeqID87) |
| 2443_SLIC_pINT_ | TAAATCGATACTAGCATAACCCCTTGGGGCCTCTAAACGCGTCGACAC |
| TP-forw | GCAAAAAGGCCATCC (SeqID88) |
| 2527_SLIC_TP_pINT- | GTTCGACAAAAATCTAGAAATAATTTTGTTTAACTTTAAGAAGGAGATAT |
| forw | ACAAGAGCTCGAGTCGAAGGAGATAGAACC (SeqID89) |
| 2444_SLIC_TP_pINT- | GGATGGCCTTTTTGCGTGTCGACGCGTTTAGAGGCCCCAAGGGGTTAT |
| rev | GCTAGTATCGATTTA (SeqID90) |
| 688 IgtA AatII rev | ATATGACGTCTCATTAGCGGTTTTTCAGGAGACG (SeqID91) |
| 689 IgtA NdeI for | ATATCATATGCCGTCCGAAGCATTCCGTCGTCACC (SeqID92) |
| 991 galT-pCDF for | TAACTTTAATAAGGAGATATACCATGACGCAATTTAATCCCGTTGATCAT |
| | CCACATCGCCGC (SeqID93) |
| 992 pCDF-galT for | ATTTTCGCGAATCCGGAGTGTAAAAGCTTGCGGCCGCATAATGCTTAAG |
| | TCGAACAGAAAGTAATCG (SeqID94) |
| 993 galT-pCDF rev | AAGCATTATGCGGCCGCAAGCTTTTACACTCCGGATTCGCGAAAATGG |
| | ATATCGCTGACTGCGCGCAAACGC (SeqID95) |
| 994 pCDF-galT rev | TCAACGGGATTAAATTGCGTCATGGTATATCTCCTTATTAAAGTTAAACA |
| | AAATTATTTCTACAGGGG (SeqID96) |
| 848 gImM pCOLA | ATGGTGATGGCTGCTGCCCATTTAAACCGCTTTGACTGCGTCGGCAATA |
| SLIC rev | CGGTGCGC (SeqID97) |
| 849 glmU pCOLA | GTTTAACTTTAATAAGGAGATATACCATGCTGAACAACGCGATGTCTGTT |
| SLIC for | GTTATCCTGG (SeqID98) |
| 850 pCOLA glmM | CGCAGTCAAAGCGGTTTAAATGGGCAGCAGCCATCACCATCATCACCA |
| SLIC rev | CAGCC (SeqID99) |
| 851 pCOLA glmU | TCGCGTTGTTCAGCATGGTATATCTCCTTATTAAAGTTAAACAAAATTAT |
| SLIC for | TTCTACAGG (SeqID100) |
| 852 glmSco pCOLA | ATATATCATATGTGCGGTATCGTTGGTGCTATCGC (SeqID101) |
| for Ndel | |
| 853 glmSco pCOLA | ATATATGACGTCTTATTCCACGGTCACGGATTTCGC (SeqID102) |
| rev AatII | |

1-24. (canceled)

25. A method for the production of lacto-N-triose II by a genetically modified microbial host cell, comprising
providing a genetically modified microbial host cell that comprises:
at least one recombinant β-1,3-N-acetylglucosaminyltransferase, wherein the at least one recombinant B-1,3-N-acetylglucosaminyltransferase belongs to the class of lgtA of Neisseria meningitidis, and
increased expression or activity of at least one sugar export protein capable of exporting the lacto-N-triose II, wherein the at least one sugar export protein is YjhB from E. coli or ProP from Mannheimia succiniciproducens;
cultivating the microbial host cell in a medium under conditions permissive for the production of the lacto-N-triose II, whereby the lacto-N-triose II is exported into the medium at an increased level compared to the unmodified microbial host cell, and
obtaining the lacto-N-triose II from the medium.

26. The method of claim 1, wherein, where applicable, in the genetically modified microbial host cell the endogenous β-galactosidase gene and the glucosamine-6-phosphate deaminase gene are inactivated or deleted, and wherein said genetically modified microbial host cell comprises a nucleic acid sequence coding for a functional lactose permease protein.

27. The method of claim 1, wherein the genetically modified host cell comprises an increased UDP-N-acetylglucosamine and UDP-galactose or GDP-fucose or CMP-N-acetylneuraminic acid production capability as compared to a genetically unmodified host cell, wherein optionally said increased UDP-N-acetylglucosamine and UDP-galactose production capability comprises the overexpression of one or more genes encoding for proteins comprising the following activities for a: L-glutamine: D-fructose-6-phosphate aminotransferase, N-acetyl glucosamine-1-phosphate uridyltransferase/glucosamine-1-phosphate acetyl transferase, phosphoglucosamine mutase, UDP-galactose-4-epimerase, phosphoglucomutase, glucose-1-phosphate uridylyltransferase.

28. The method of claim 1, wherein said genetically modified microbial host cell is cultivated in the presence of glucose, sucrose, glycerol or a combination thereof, but neither by addition or in the presence of N-acetylglucosamine or galactose nor in a combination thereof.

29. A genetically modified microbial host cell for the production of lacto-N-triose II, wherein the microbial host cell comprises:
a. at least one recombinant β-1,3-N-acetylglucosaminyltransferase, wherein the at least one recombinant B-1,3-N-acetylglucosaminyltransferase belongs to the class of lgtA of Neisseria meningitidis, and
b. increased expression or activity of at least one sugar export protein capable of exporting the lacto-N-triose II, wherein the at least one sugar export protein is YjhB from E. coli or ProP from Mannheimia succiniciproducens.

30. The genetically modified microbial host cell of claim 5, wherein, where applicable, in the genetically modified microbial host cell the endogenous β-galactosidase gene and the glucosamine-6-phosphate deaminase gene are inactivated or deleted, and wherein said genetically modified microbial host cell comprises a nucleic acid sequence coding for a functional lactose permease protein.

31. The genetically modified microbial host cell of claim 5, wherein the genetically modified host cell comprises an increased UDP-N-acetylglucosamine and UDP-galactose or GDP-fucose or CMP-N-acetylneuraminic acid production capability as compared to a genetically unmodified host cell, wherein optionally said increased UDP-N-acetylglucosamine and UDP-galactose production capability comprises the overexpression of one or more genes encoding for proteins comprising the following activities for a: L-glutamine: D-fructose-6-phosphate aminotransferase, N-acetyl glucosamine-1-phosphate uridyltransferase/glucosamine-1-phosphate acetyl transferase, phosphoglucosamine mutase, UDP-galactose-4-epimerase, phosphoglucomutase, glucose-1-phosphate uridylyltransferase.


SEQUENCE LISTING (102 sequences)

SEQ ID NO: 1 - DNA, 2851 bp, synthetic construct
  misc_feature 1..2851: note = construct Ptet-lacY-FRT-add1-FRT
  source 1..2851: mol_type = other DNA
tggccagatgattaattcctaatttttgttgacactctatcattgatagagttattttaccactccctatcagtgatagagaaaagtgaaatgaatagttcgacaaaaatctagaaataattttgtttaactttaagaaggagatatacaaatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcgggctgcgcaaatacctgctgtggattattaccggcatgttagtgatgtttgcgccgttctttatttttatcttcgggccactgttacaatacaacattttagtaggatcgattgttggtggtatttatctaggcttttgttttaacgccggtgcgccagcagtagaggcatttattgagaaagtcagccgtcgcagtaatttcgaatttggtcgcgcgcggatgtttggctgtgttggctgggcgctgtgtgcctcgattgtcggcatcatgttcaccatcaataatcagtttgttttctggctgggctctggctgtgcactcatcctcgccgttttactctttttcgccaaaacggatgcgccctcttctgccacggttgccaatgcggtaggtgccaaccattcggcatttagccttaagctggcactggaactgttcagacagccaaaactgtggtttttgtcactgtatgttattggcgtttcctgcacctacgatgtttttgaccaacagtttgctaatttctttacttcgttctttgctaccggtgaacagggtacgcgggtatttggctacgtaacgacaatgggcgaattacttaacgcctcgattatgttctttgcgccactgatcattaatcgcatcggtgggaaaaacgccctgctgctggctggcactattatgtctgtacgtattattggctcatcgttcgccacctcagcgctggaagtggttattctgaaaacgctgcatatgtttgaagtaccgttcctgctggtgggctgctttaaatatattaccagccagtttgaagtgcgtttttcagcgacgatttatctggtctgtttctgcttctttaagcaactggcgatgatttttatgtctgtactggcgggcaatatgtatgaaagcatcggtttccagggcgcttatctggtgctgggtctggtggcgctgggcttcaccttaatttccgtgttcacgcttagcggccccggcccgctttccctgctgcgtcgtcaggtgaatgaagtcgctgggagctaagcggccgcgtcgacacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcatgtatgaatatcctccttagttcctattccgaagttcctattctctagaaagtataggaacttcggcgcgtcctacctgtgacacgcgtgccgcagtctcacgcccggagcgtagcgaccgagtgagctagctatttgtttatttttctaaatacattcaaatatgtatccgctcatgagacaataaccctgataaatgcttcaataatattgaaaaaggaagagtatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgaggggccgcaagatccggccacgatgacccggtcgtcgggtaccggcagggcggggcgtaaggcgcgccatttaaatgaagttcctattccgaagttcctattctctagaaagtataggaactt

SEQ ID NO: 2 - DNA, 4568 bp, synthetic construct
  misc_feature 1..4568: note = construct Ptet-lgtA-PT5-galT-FRT-kanR-FRT
  source 1..4568: mol_type = other DNA
acaggttggctgataagtccccggtctagcttgcatgcagattgcagcattacacgtcttgagcgattgtgtaggctggagctgcttcgaagttcctatactttctagagaataggaacttcggaataggaacttcatttaaatggcgcgccttacgccccgccctgccggtaccgagagcgcttttgaagctggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaggggatcttgacgcgtgtcacaggtaggacgcgccgaagttcctatactttctagagaataggaacttcggaataggaactaaggaggatattcatacatgatggtagtgttcgaaattaatacgactcactataggggaattgattctggtaccaaatgagtcgaccggccagatgattaattcctaatttttgttgacactctatcattgatagagttattttaccactccctatcagtgatagagaaaagtgaaatgaatagttcgacaaaaatctagaaataattttgtttaactttaagaaggagatatacaaatgccgtccgaagcattccgtcgtcaccgtgcttatcgcgaaaacaaactgcagccactggtctctgtcctgatctgcgcatacaacgttgagaaatacttcgcacagtctctggcagctgtagttaaccagacctggcgtaacctggatatcctgatcgtagatgacggctctacggatggtacgctggcgatcgcacagcgtttccaggaacaggacggtcgtatccgcattctcgctcagccgcgtaactctggtctgatcccgtctctgaacatcggtctggacgaactggccaaatctggtggtggtggcgaatacatcgcccgtactgacgccgacgacattgcggccccggattggatcgaaaaaatcgtaggtgaaatggagaaagaccgctctatcatcgcgatgggtgcttggctggaagttctgtccgaagagaaagacggtaaccgtctggcccgtcaccatgaacacggcaaaatctggaaaaaaccgacccgtcacgaagatatcgcggacttcttcccgttcggtaacccgatccataacaacaccatgatcatgcgtcgtagcgtaatcgacggtggtctgcgttacaacaccgaacgtgattgggcagaagactaccagttttggtatgacgtgtctaaactgggtcgtctggcttactacccagaagcgctggttaaataccgtctgcacgccaaccaggttagctccaaatactccatccgtcagcacgaaatcgcacagggtatccagaaaacggctcgtaacgacttcctgcagtccatgggtttcaaaacccgtttcgactctctggagtaccgtcagatcaaagcggttgcgtatgagctgctggagaaacacctgccggaagaggactttgaacgtgcgcgtcgtttcctgtaccagtgcttcaaacgtaccgacactctgccggcgggtgcatggctcgactttgcagcggatggtcgtatgcgtcgtctgtttaccctgcgtcagtacttcggtatcctgcatcgtctcctgaaaaaccgctaatgatttcgtcgacacacaggaaacatattaaaaattaaaacctgcaggagtttaaacgcggccgcgatatcgttgtaaaacgacggccagtgcaagaatcataaaaaatttatttgctttcaggaaaatttttctgtataatagattcataaatttgagagaggagtttttgtgagcggataacaattccccatcttagtatattagttaagtataaatacacaaggagatataccatgacgcaatttaatcccgttgatcatccacatcgccgctacaacccgctcaccgggcaatggattctggtttcaccgcaccgcgctaagcgcccctggcagggggcgcaggaaacgccagccaaacaggtgttacctgcgcacgatccagattgcttcctctgcgcaggtaatgtgcgggtgacaggcgataaaaaccccgattacaccgggacttacgttttcactaatgactttgcggctttgatgtctgacacgccagatgcgccagaaagtcacgatccgctgatgcgttgccagagcgcgcgcggcaccagccgggtgatctgcttttcaccggatcacagtaaaacgctgccagagctcagcgttgcagcattgacggaaatcgtcaaaacctggcaggagcaaaccgcagaactggggaaaacgtacccatgggtgcaggtttttgaaaacaaaggcgcggcgatgggctgctctaacccgcatccgcacggtcagatttgggcaaatagcttcctgcctaacgaagctgagcgcgaagaccgcctgcaaaaagaatattttgccgaacagaaatcaccaatgctggtggattatgttcagcgcgagctggcagacggtagccgtaccgttgtcgaaaccgaacactggttagccgtcgtgccttactgggctgcctggccgttcgaaacgctactgctgcccaaagcccacgttttacggatcaccgatttgaccgacgcccagcgcagcgatctggcgctggcgttgaaaaagctgaccagtcgttatgacaacctcttccagtgctccttcccctactctatgggctggcacggcgcgccatttaatggcgaagagaatcaacactggcagctgcacgcgcacttttatccgcctctgctgcgctccgccaccgtacgtaaatttatggttggttatgaaatgctggcagagacccagcgagacctgaccgcagaacaggcagcagagcgtttgcgcgcagtcagcgatatccattttcgcgaatccggagtgtaacgcggaggcgcgccatttaaatcaacctcagcggtcatagctgtttcctgtgactgagcaataactagcataaccccttggggcctctaaacgggtcttgaggggttttttgctgaaaccaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgggatccaggccggcctgttaacgaattaatcttccgcggcaacaaaaattaggaattaatcatctggccaatttcaggtggcacttttcgggcagaccggggacttatcagccaacctgt

SEQ ID NO: 3 - DNA, 6521 bp, synthetic construct
  misc_feature 1..6521: note = construct Ptet-glmUM-PT5-glmS-FRT-dhfr-FRT
  source 1..6521: mol_type = other DNA
acaggttggctgataagtccccggtctagcttgcatgcagattgcagcattacacgtcttgagcgattgtgtaggctggagctgcttcgaaattaatacgactcactataggggaattgattctggtaccaaatgagtcgaccggccagatgattaattcctaatttttgttgacactctatcattgatagagttattttaccactccctatcagtgatagagaaaagtgaaatgaatagttcgacaaaaatctagaaataattttgtttaactttaagaaggagatatacaaatgctgaacaacgcgatgtctgttgttatcctggcggcgggtaaaggtacccgtatgtactctgacctgccgaaagttctgcacaccctggcgggtaaagcgatggttcagcacgttatcgacgcggcgaacgaactgggtgcggcgcacgttcacctggtttacggtcacggtggtgacctgctgaaacaggcgctgaaagacgacaacctgaactgggttctgcaggcggaacagctgggtaccggtcacgcgatgcagcaggcggcgccgttcttcgcggacgacgaagacatcctgatgctgtacggtgacgttccgctgatctctgttgaaaccctgcagcgtctgcgtgacgcgaaaccgcagggtggtatcggtctgctgaccgttaaactggacgacccgaccggttacggtcgtatcacccgtgaaaacggtaaagtaaccggtatcgttgaacacaaagacgcgaccgacgaacagcgtcagatccaggagatcaacaccggtatcctgatcgcgaacggtgcagacatgaaacgttggctggcgaaactgaccaacaacaacgcgcagggtgaatactacatcaccgacatcatcgcgctggcgtaccaggaaggtcgtgaaatcgttgcggttcacccgcagcgtctgtctgaagttgaaggtgttaacaaccgtctgcagctgtctcgtctggaacgtgtttaccagtctgaacaggcggaaaaactgctgctggcgggtgttatgctgcgtgacccggcgcgtttcgacctgcgtggtaccctgacccacggtcgtgacgttgaaatcgacaccaacgttatcatcgaaggtaacgttaccctgggtcaccgtgtaaaaatcggcaccggttgcgttatcaaaaactctgttatcggtgacgactgcgaaatctctccgtacaccgttgttgaagacgcgaacctggcggcggcgtgcaccatcggtccgttcgcgcgtctgcgtccgggtgcggaactgctggaaggtgcgcacgttggtaacttcgttgaaatgaaaaaagcgcgtctgggtaaaggttctaaagcgggtcacctgacctacctgggtgacgcggaaatcggtgacaacgttaacatcggtgcgggtaccatcacctgcaactacgacggtgcgaacaaattcaaaaccatcatcggtgacgacgttttcgttggttctgacacccagctggttgcgccggttaccgttggtaaaggtgcgaccatcgcggcgggtaccaccgttacccgtaacgttggtgaaaacgcgctggcgatctctcgtgttccgcagacccagaaagaaggttggcgtcgtccggttaaaaaaaaataacgaaggagatagaaccatgtccaaccgtaaatacttcggtacggacggtatccgtggtcgtgtaggtgatgctccgattacgccggatttcgtcctgaaactcggttgggcagcgggtaaagttctcgcacgtcacggctctcgtaaaatcatcatcggtaaagacacccgtatctctggttacatgctcgaatctgcactggaagcgggtctggctgcagctggtctgtctgcactgttcacgggtccgatgccaaccccagctgtagcgtacctgactcgcactttccgtgcagaagcaggtatcgtgatctctgcctctcacaacccgttctacgacaacggtatcaaattcttcagcatcgatggtaccaaactcccagacgcggttgaagaggctatcgaagcggaaatggagaaagaaatctcttgtgtagactctgccgaactcggtaaagcgtctcgtatcgttgatgcagcgggtcgttacatcgagttctgcaaagccacctttccgaacgaactgagcctgtctgagctgaaaatcgtcgtagactgtgccaacggtgcgacttaccacattgccccaaacgtactgcgtgagctgggtgctaacgtcatcgcgatcggttgtgaaccgaacggtgtcaacatcaacgcggaagtaggtgcgaccgatgttcgtgcactgcaggctcgtgtactcgcggagaaagcggatctcggtatcgcctttgacggtgatggtgaccgtgttatcatggttgaccacgaaggtaacaaagtggatggtgaccagatcatgtacatcattgcccgtgaaggtctgcgtcagggtcagctgcgtggtggtgcagtaggtaccctcatgagcaacatgggtctggaactggccctgaaacagctgggtatcccattcgctcgtgctaaagtaggcgaccgttacgttctggagaaaatgcaggagaaaggttggcgtatcggtgccgaaaactctggtcacgtcatcctgctggacaaaaccactaccggtgacggtatcgtagcaggtctgcaggtactcgccgctatggcccgtaaccacatgtccctccatgacctctgctctggtatgaaaatgttcccgcagatcctggttaacgttcgttacaccgcaggttctggtgatccgctggaacacgagtctgtgaaagccgttaccgcagaagtggaagcggccctgggtaaccgtggtcgtgtactgctgcgtaaatccggtactgagccactgatccgtgttatggttgagggcgaagatgaagcccaggtcaccgaatttgcgcaccgtattgccgacgcagtcaaagcggtttaatttcgtcgacacacaggaaacatattaaaaattaaaacctgcaggagtttaaacgcggccgcgatatcgttgtaaaacgacggccagtgcaagaatcataaaaaatttatttgctttcaggaaaatttttctgtataatagattcataaatttgagagaggagtttttgtgagcggataacaattccccatcttagtatattagttaagtataaatacacaaggagatatacatatgtgcggtatcgttggtgctatcgcacagcgtgatgtagcggagatcctcctggaaggtctgcgtcgtctcgaataccgtggttacgactctgccggtctggcagtagtggatgcagaaggtcacatgactcgtctgcgtcgtctgggtaaagtgcagatgctcgcgcaggcggcggaagaacacccactccacggtggtacgggtatcgcacacactcgttgggcaacccacggtgaaccgtctgaggtcaacgcacacccgcatgttagcgagcacatcgtagtcgttcacaacggtatcatcgagaaccacgaaccactccgtgaggaactcaaagcccgtggttacaccttcgtaagcgaaaccgacacggaagttatcgcccacctcgttaactgggaactcaaacagggtggtactctgcgtgaagcagttctgcgtgccattccacagctgcgtggtgcatacggtaccgtgatcatggactctcgtcatccggataccctgctcgccgcacgttctggttctccactcgttatcggtctgggtatgggtgagaacttcatcgcctctgatcagctggccctgctcccagttacccgtcgcttcatcttcctggaagagggtgacatcgccgaaatcacccgtcgttccgttaacatcttcgacaaaacgggtgcggaagttaaacgtcaggacatcgagtctaacctgcagtatgacgctggtgacaaaggcatctaccgtcactacatgcagaaagagatctacgaacagccgaacgcgatcaaaaacaccctgaccggtcgtatctctcacggtcaggttgacctgtctgagctgggtccaaacgcggacgaactcctgtccaaagtcgagcacatccagatcctggcttgtggtacctcttacaactccggtatggtttctcgttactggttcgaatctctggcaggtatcccatgcgacgttgaaatcgcctccgaattccgttatcgtaaatctgcggtacgtcgtaactccctcatgatcaccctgtctcagtctggtgaaaccgctgatactctggcaggtctgcgtctcagcaaagaactgggttacctgggttctctggccatctgcaacgttccgggttctagcctggttcgtgagtctgacctggctctgatgaccaacgcgggtacggagatcggtgttgcctctaccaaagcgttcactacccagctcactgtcctgctgatgctggttgccaaactgtctcgtctcaaaggcctcgacgctagcatcgaacacgacatcgtacacggtctgcaggccctcccatctcgtatcgagcagatgctgtctcaggacaaacgtatcgaagcactggcagaagacttcagcgacaaacaccacgcgctgtttctgggtcgtggtgaccagtacccaattgcgctggaaggtgccctgaaactgaaagagatcagctacatccatgcagaggcatacgcagcgggtgagctgaaacatggtccactggccctgatcgacgcagatatgccggttattgtggttgctccgaacaacgaactgctggagaaactgaaatccaacatcgaggaagtacgtgcgcgtggtggtcagctgtacgtgtttgctgaccaggacgcgggtttcgtttccagcgacaacatgcacatcatcgaaatgccgcatgttgaagaggtaatcgcgccaatcttctacaccgtaccgctgcagctgctggcgtaccatgtagccctgatcaaaggtacggacgttgaccagccgcgtaacctggcgaaatccgtgaccgtggaataacgcggaggcgcgccatttaaatcaacctcagcggtcatagctgtttcctgtgactgagcaataactagcataaccccttggggcctctaaacgggtcttgaggggttttttgctgaaaccaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgggatccaggccggcctgttaacgaattaatcttccgcggcggtatcgataagcttgatatcgaattccgaagttcctattctctagaaagtataggaacttcaggtctgaagaggagtttacgtccagccaagctagcttggctgcaggtcgtcgaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcctgttgacaattaatcatcggcatagtatatcggcatagtataatacgacaaggtgaggaactaaaccatgggtcaaagtagcgatgaagccaacgctcccgttgcagggcagtttgcgcttcccctgagtgccacctttggcttaggggatcgcgtacgcaagaaatctggtgccgcttggcagggtcaagtcgtcggttggtattgcacaaaactcactcctgaaggctatgcggtcgagtccgaatcccacccaggctcagtgcaaatttatcctgtggctgcacttgaacgtgtggcctaatgaggggatcaattctctagagctcgctgatcagaagttcctattctctagaaagtataggaacttcgatggcgcctcatccctgaagccaatacaacaaaaattaggaattaatcatctggccaatttcaggtggcacttttcgggcagaccggggacttatcagccaacctgt

SEQ ID NO: 4 - DNA, 2937 bp, synthetic construct
  source 1..2937: mol_type = other DNA
  misc_feature 1..2937: note = construct <Ptet-lacY(6HIS)-FRT-aadA-FRT
ggccagatgattaattcctaatttttgttgacactctatcattgatagagttattttaccactccctatcagtgatagagaaaagtgaaatgaatagttcgacaaaaatctagaaataattttgtttaactttaagaaggagatatacaaatgggctactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcgggctgcgcaaatacctgctgtggattattaccggcatgttagtgatgtttgcgccgttctttatttttatcttcgggccactgttacaatacaacattttagtaggatcgattgttggtggtatttatctaggcttttgttttaacgccggtgcgccagcagtagaggcatttattgagaaagtcagccgtcgcagtaatttcgaatttggtcgcgcgcggatgtttggctgtgttggctgggcgctgtgtgcctcgattgtcggcatcatgttcaccatcaataatcagtttgttttctggctgggctctggctgtgcactcatcctcgccgttttactctttttcgccaaaacggatgcgccctcttctcatcaccatcaccatcacgccacggttgccaatgcggtaggtgccaaccattcggcatttagccttaagctggcactggaactgttcagacagccaaaactgtggtttttgtcactgtatgttattggcgtttcctgcacctacgatgtttttgaccaacagtttgctaatttctttacttcgttctttgctaccggtgaacagggtacgcgggtatttggctacgtaacgacaatgggcgaattacttaacgcctcgattatgttctttgcgccactgatcattaatcgcatcggtgggaaaaacgccctgctgctggctggcactattatgtctgtacgtattattggctcatcgttcgccacctcagcgctggaagtggttattctgaaaacgctgcatatgtttgaagtaccgttcctgctggtgggctgctttaaatatattaccagccagtttgaagtgcgtttttcagcgacgatttatctggtctgtttctgcttctttaagcaactggcgatgatttttatgtctgtactggcgggcaatatgtatgaaagcatcggtttccagggcgcttatctggtgctgggtctggtggcgctgggcttcaccttaatttccgtgttcacgcttagcggccccggcccgctttccctgctgcgtcgtcaggtgaatgaagtcgcttaagcggccgcgtcgacacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcatgtatgaatatcctccttagttcctattccgaagttcctattctctagaaagtataggaacttcggcgcgtcctacctgtgacacgcgtgccgcagtctcacgcccggagcgtagcgaccgagtgagctagctatttgtttatttttctaaatacattcaaatatgtatccgctcatgagacaataaccctgataaatgcttcaataatattgaaaaaggaagagtatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgaggggccgcaagatccggccacgatgacccggtcgtcgggtaccggcagggcggggcgtaaggcgcgccatttaaatgaagttcctattccgaagttcctattctctagaaagtataggaacttcgaagcagctccagcctacacaatcgctcaagacgtgtaatgctgcaatctgcatgcaagcttggcactggc

SEQ ID NO: 5 - DNA, 3856 bp, synthetic construct
  source 1..3856: mol_type = other DNA
  misc_feature 1..3856: note = Ptet-wbdO-PT5-galE-FRT-cat-FRT
acaggttggctgataagtccccggtctgcccgaaaagtgccacctgaaattggccagatgattaattcctaatttttgttgattctggtaccaaatgagtcgaccggccagatgattaattcctaatttttgttgacactctatcattgatagagttattttaccactccctatcagtgatagagaaaagtgaaatgaatagttcgacaaaaatctagaaataattttgtttaactttaagaaggagatatacaaatgctgacggaagtgcgcccggtctctacgacgaaaccgctggtgtctgtgattctgccggtgaacaaattcaacccgtatctggatcgtgcaattcattcaatcctgagtcagtcctatccgtcgattgaactgattatcattgcaaacaattgcaccaatgactttttcgatgctctgaaaaaacgtgaatgtgaaaccattaaagtgctgcgcacgaacatcgcgtatctgccgtactgcctgaataaaggcctggatctgtgtaacggtgactttgttgcccgcatggattcagatgacatttcgcacccggaacgtatcgatcgccaggtcgacttcctgattaacaatccggacatcgatgtggttggcaccaatgcagtctatattgatgaagatgacatcgaactggaaaaaagcaacctgccggtgaacaataacgctattcgtaaaatgctgccgtataaatgctgtctggtgcatccgtctgttatgtttcgcaaaaatgtcgtgatcaccagcggcggttacatgttcgcgaattattctgaagattacgaactgtggaaccgtctggccgttgaaggccgcaatttttataacctgagcgaatacctgctgtattaccgtctgcacaataaccaatcaacgtcgaaaaataacctgtttatggtgatggcgaacgatgtcgccattaaagtgaaatatttcctgctgaccaagaaaattagctacctgctgggtatcattcgcacggtcttttctgtgttctattgcaaatacatcaaatgatttcgtcgacacacaggaaacatattaaaaattaaaacctgcaggagtttaaacgcggccgcgatatcgttgtaaaacgacggccagtgcaagaatcataaaaaatttatttgctttcaggaaaatttttctgtataatagattcataaatttgagagaggagtttttgtgagcggataacaattccccatcttagtatattagttaagtataaatacaccgcggaggcgtcgaaggagatacaaccatgagagttctggttaccggtggtagcggttacattggaagtcatacctgtgtgcaattactgcaaaacggtcatgatgtcatcattcttgataacctctgtaacagtaagcgcagcgtactgcctgttatcgagcgtttaggcggcaaacatccaacgtttgttgaaggcgatattcgtaacgaagcgttgatgaccgagatcctgcacgatcacgctatcgacaccgtgatccacttcgccgggctgaaagccgtgggcgaatcggtacaaaaaccgctggaatattacgacaacaatgtcaacggcactctgcgcctgattagcgccatgcgcgccgctaacgtcaaaaactttatttttagctcctccgccaccgtttatggcgatcagcccaaaattccatacgttgaaagcttcccgaccggcacaccgcaaagcccttacggcaaaagcaagctgatggtggaacagatcctcaccgatctgcaaaaagcccagccggactggagcattgccctgctgcgctacttcaacccggttggcgcgcatccgtcgggcgatatgggcgaagatccgcaaggcattccgaataacctgatgccatacatcgcccaggttgctgtaggccgtcgcgactcgctggcgatttttggtaacgattatccgaccgaagatggtactggcgtacgcgattacatccacgtaatggatctggcggacggtcacgtcgtggcgatggaaaaactggcgaacaagccaggcgtacacatctacaacctcggcgctggcgtaggcaacagcgtgctggacgtggttaatgccttcagcaaagcctgcggcaaaccggttaattatcattttgcaccgcgtcgcgagggcgaccttccggcctactgggcggacgccagcaaagccgaccgtgaactgaactggcgcgtaacgcgcacactcgatgaaatggcgcaggacacctggcactggcagtcacgccatccacagggatatcccgattaacgccatttaaatcaacctcagcggtcatagctgtttcctgtgactgagcaataactagcataaccccttggggcctctaaacgggtcttgaggggttttttgctgaaaccaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgggatccaggccggcctgttaacgaattaatcttccgcggcggtatcgataagcttgatatcgaggctgacatgggaattagccatggtccatatgaatatcctccttagttcctattccgaagttcctattctctagaaagtataggaacttcggcgcgcctacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagatgcttaatgaatacaacagtactgcgatgagtggcagggcggggcgtaaggcgcgccatttaaatgaagttcctattccgaagttcctattctctagaaagtataggaacttcgaagcagctccagcctacacaatcgctcaagacgtgtaatgctgcaatctgcatgcaagcttggcactggcgatggcgcctcatccctgaagccaataagcagctccagcctacacaatcgctcaagacgtgtaatgctgcaatctgcatgcaagctagaccggggacttatcagccaacctgt

SEQ ID NO: 6 - DNA, 4259 bp, synthetic construct
  misc_feature 1..4259: note = construct galMKTE
  source 1..4259: mol_type = other DNA
ttactcagcaataaactgatattccgtcaggctggaatactcttcgccaggacgcaggaagcagtccggttgcggccattcagggtggttcgggctgtccggtagaaactcgctttccagagccagcccttgccagtcggcgtaaggttcggttccccgcgacggtgtgccgccgaggaagttgccggagtagaattgcagagccggagcggtggtgtagaccttcagctgcaatttttcatctgctgaccagacatgcgccgccactttcttgccatcgcctttggcctgtaacaagaatgcgtgatcgtaacctttcactttgcgctgatcgtcgtcggcaagaaactcactggcgatgattttggcgctgcggaaatcaaaagacgttccggcgacagatttcaggccgtcgtgcggaatgccgccttcatcaaccggcagatattcgtccgccagaatctgcaacttgtgattgcgcacgtcagactgctcgccgtcaagattgaaatagacgtgattagtcatattcaccgggcaaggtttatcaactgtggcgcgataagtaatggagatacggttatcgtcggtcagacgatattgcaccgtcgcgccgagattacccgggaagccctgatcaccatcatctgaactcagggcaaacagcacctgacgatcgttctggttcacaatctgccagcgacgtttgtcgaacccttccggcccgccgtgcagctggttaacgccctgacttggcgaaagcgtcacggtttcaccgtcaaaggtataacggctattggcgatacggttggcataacgaccaatagaggcccccagaaacgcggcctgatcctgatagcattccgggctggcacagccgagcagcgcctcgcggacgctgccatcggaaagcggaatacgggcggaaagtaaagtcgcaccccagtccatcagcgtgactaccatccctgcgttgttacgcaaagttaacagtcggtacggctgaccatcgggtgccagtgcgggagtttcgttcagcactgtcctgctccttgtgatggtttacaaacgtaaaaagtctctttaatacctgtttttgcttcatattgttcagcgacagcttgctgtacggcaggcaccagctcttccgggatcagcgcgacgatacagccgccaaatccgccgccggtcatgcgtacgccacctttgtcgccaatcacagctttgacgatttctaccagagtgtcaatttgcggcacggtgatttcgaaatcatcgcgcatagaggcatgagactccgccatcaactcgcccatacgtttcaggtcgccttgctccagcgcgctggcagcttcaacggtgcgggcgttttcagtcagtatatgacgcacgcgttttgccacgatcgggtccagttcatgcgcaacagcgttgaactcttcaatggtgacatcacgcagggctggctgctggaagaaacgcgcaccggtttcgcactgttcacgacgggtgttgtattcgctgccaaccagggtacgtttgaagttactgttgatgatgacgacagccacacctttgggcatggaaactgctttggtccccagtgagcggcaatcgatcagcaaggcatgatctttcttgccgagcgcggaaattagctgatccatgatcccgcagttacagcctacaaactggttttctgcttcctgaccgttaagcgcgatttgtgcgccgtccagcggcagatgataaagctgctgcaatacggttccgaccgcgacttccagtgaagcggaagaacttaacccggcaccctgcggcacattgccgctgatcaccatgtccacgccgccgaagctgttgttacgcagttgcagatgtttcaccacgccacgaacgtagttagcccattgatagttttcatgtgcgacaatgggcgcatcgagggaaaactcgtcgagctgattttcataatcggctgccatcacgcgaactttacggtcatcgcgtggtgcacaactgatcacggtttgataatcaatcgcgcagggcagaacgaaaccgtcgttgtagtcggtgtgttcaccaatcaaattcacgcggccaggcgcctgaatggtgtgagtggcagggtagccaaatgcgttggcaaacagagattgtgttttttctttcagactcatttcttacactccggattcgcgaaaatggatatcgctgactgcgcgcaaacgctctgctgcctgttctgcggtcaggtctcgctgggtctctgccagcatttcataaccaaccataaatttacgtacggtggcggagcgcagcagaggcggataaaagtgcgcgtgcagctgccagtgttgattctcttcgccattaaatggcgcgccgtgccagcccatagagtaggggaaggagcactggaagaggttgtcataacgactggtcagctttttcaacgccagcgccagatcgctgcgctgggcgtcggtcaaatcggtgatccgtaaaacgtgggctttgggcagcagtagcgtttcgaacggccaggcagcccagtaaggcacgacggctaaccagtgttcggtttcgacaacggtacggctaccgtctgccagctcgcgctgaacataatccaccagcattggtgatttctgttcggcaaaatattctttttgcaggcggtcttcgcgctcagcttcgttaggcaggaagctatttgcccaaatctgaccgtgcggatgcgggttagagcagcccatcgccgcgcctttgttttcaaaaacctgcacccatgggtacgttttccccagttctgcggtttgctcctgccaggttttgacgatttccgtcaatgctgcaacgctgagctctggcagcgttttactgtgatccggtgaaaagcagatcacccggctggtgccgcgcgcgctctggcaacgcatcagcggatcgtgactttctggcgcatctggcgtgtcagacatcaaagccgcaaagtcattagtgaaaacgtaagtcccggtgtaatcggggtttttatcgcctgtcacccgcacattacctgcgcagaggaagcaatctggatcgtgcgcaggtaacacctgtttggctggcgtttcctgcgccccctgccaggggcgcttagcgcggtgcggtgaaaccagaatccattgcccggtgagcgggttgtagcggcgatgtggatgatcaacgggattaaattgcgtcatggtcgttccttaatcgggatatccctgtggatggcgtgactgccagtgccaggtgtcctgcgccatttcatcgagtgtgcgcgttacgcgccagttcagttcacggtcggctttgctggcgtccgcccagtaggccggaaggtcgccctcgcgacgcggtgcaaaatgataattaaccggtttgccgcaggctttgctgaaggcattaaccacgtccagcacgctgttgcctacgccagcgccgaggttgtagatgtgtacgcctggcttgttcgccagtttttccatcgccacgacgtgaccgtccgccagatccattacgtggatgtaatcgcgtacgccagtaccatcttcggtcggataatcgttaccaaaaatcgccagcgagtcgcgacggcctacagcaacctgggcgatgtatggcatcaggttattcggaatgccttgcggatcttcgcccatatcgcccgacggatgcgcgccaaccgggttgaagtagcgcagcagggcaatgctccagtccggctgggctttttgcagatcggtgaggatctgttccaccatcagcttgcttttgccgtaagggctttgcggtgtgccggtcgggaagctttcaacgtatggaattttgggctgatcgccataaacggtggcggaggagctaaaaataaagtttttgacgttagcggcgcgcatggcgctaatcaggcgcagagtgccgttgacattgttgtcgtaatattccagcggtttttgtaccgattcgcccacggctttcagcccggcgaagtggatcacggtgtcgatagcgtgatcgtgcaggatctcggtcatcaacgcttcgttacgaatatcgccttcaacaaacgttggatgtttgccgcctaaacgctcgataacaggcagtacgctgcgcttactgttacagaggttatcaagaatgatgacatcatgaccgttttgcagtaattgcacacaggtatgacttccaatgtaaccgctaccaccggtaaccagaactctcat

SEQ ID NO: 7 - DNA, 6431 bp, synthetic construct
  misc_feature 1..6431: note = construct pET-lgtA
  source 1..6431: mol_type = other DNA
ggggaattgtgagcggataacaattcccctctagaaataattttgtttaactttaagaaggagatataccatgggcagcagccatcaccatcatcaccacagccaggatccgaattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgccgtccgaagcattccgtcgtcaccgtgcttatcgcgaaaacaaactgcagccactggtctctgtcctgatctgcgcatacaacgttgagaaatacttcgcacagtctctggcagctgtagttaaccagacctggcgtaacctggatatcctgatcgtagatgacggctctacggatggtacgctggcgatcgcacagcgtttccaggaacaggacggtcgtatccgcattctcgctcagccgcgtaactctggtctgatcccgtctctgaacatcggtctggacgaactggccaaatctggtggtggtggcgaatacatcgcccgtactgacgccgacgacattgcggccccggattggatcgaaaaaatcgtaggtgaaatggagaaagaccgctctatcatcgcgatgggtgcttggctggaagttctgtccgaagagaaagacggtaaccgtctggcccgtcaccatgaacacggcaaaatctggaaaaaaccgacccgtcacgaagatatcgcggacttcttcccgttcggtaacccgatccataacaacaccatgatcatgcgtcgtagcgtaatcgacggtggtctgcgttacaacaccgaacgtgattgggcagaagactaccagttttggtatgacgtgtctaaactgggtcgtctggcttactacccagaagcgctggttaaataccgtctgcacgccaaccaggttagctccaaatactccatccgtcagcacgaaatcgcacagggtatccagaaaacggctcgtaacgacttcctgcagtccatgggtttcaaaacccgtttcgactctctggagtaccgtcagatcaaagcggttgcgtatgagctgctggagaaacacctgccggaagaggactttgaacgtgcgcgtcgtttcctgtaccagtgcttcaaacgtaccgacactctgccggcgggtgcatggctcgactttgcagcggatggtcgtatgcgtcgtctgtttaccctgcgtcagtacttcggtatcctgcatcgtctcctgaaaaaccgctaatgagacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 8 - DNA, 5739 bp, synthetic construct
  misc_feature 1..5739: note = construct pCDF-galT-galE
  source 1..5739: mol_type = other DNA
ggggaattgtgagcggataacaattcccctgtagaaataattttgtttaactttaataaggagatataccatgacgcaatttaatcccgttgatcatccacatcgccgctacaacccgctcaccgggcaatggattctggtttcaccgcaccgcgctaagcgcccctggcagggggcgcaggaaacgccagccaaacaggtgttacctgcgcacgatccagattgcttcctctgcgcaggtaatgtgcgggtgacaggcgataaaaaccccgattacaccgggacttacgttttcactaatgactttgcggctttgatgtctgacacgccagatgcgccagaaagtcacgatccgctgatgcgttgccagagcgcgcgcggcaccagccgggtgatctgcttttcaccggatcacagtaaaacgctgccagagctcagcgttgcagcattgacggaaatcgtcaaaacctggcaggagcaaaccgcagaactggggaaaacgtacccatgggtgcaggtttttgaaaacaaaggcgcggcgatgggctgctctaacccgcatccgcacggtcagatttgggcaaatagcttcctgcctaacgaagctgagcgcgaagaccgcctgcaaaaagaatattttgccgaacagaaatcaccaatgctggtggattatgttcagcgcgagctggcagacggtagccgtaccgttgtcgaaaccgaacactggttagccgtcgtgccttactgggctgcctggccgttcgaaacgctactgctgcccaaagcccacgttttacggatcaccgatttgaccgacgcccagcgcagcgatctggcgctggcgttgaaaaagctgaccagtcgttatgacaacctcttccagtgctccttcccctactctatgggctggcacggcgcgccatttaatggcgaagagaatcaacactggcagctgcacgcgcacttttatccgcctctgctgcgctccgccaccgtacgtaaatttatggttggttatgaaatgctggcagagacccagcgagacctgaccgcagaacaggcagcagagcgtttgcgcgcagtcagcgatatccattttcgcgaatccggagtgtaaaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacagatcacatatgagagttctggttaccggtggtagcggttacattggaagtcatacctgtgtgcaattactgcaaaacggtcatgatgtcatcattcttgataacctctgtaacagtaagcgcagcgtactgcctgttatcgagcgtttaggcggcaaacatccaacgtttgttgaaggcgatattcgtaacgaagcgttgatgaccgagatcctgcacgatcacgctatcgacaccgtgatccacttcgccgggctgaaagccgtgggcgaatcggtacaaaaaccgctggaatattacgacaacaatgtcaacggcactctgcgcctgattagcgccatgcgcgccgctaacgtcaaaaactttatttttagctcctccgccaccgtttatggcgatcagcccaaaattccatacgttgaaagcttcccgaccggcacaccgcaaagcccttacggcaaaagcaagctgatggtggaacagatcctcaccgatctgcaaaaagcccagccggactggagcattgccctgctgcgctacttcaacccggttggcgcgcatccgtcgggcgatatgggcgaagatccgcaaggcattccgaataacctgatgccatacatcgcccaggttgctgtaggccgtcgcgactcgctggcgatttttggtaacgattatccgaccgaagatggtactggcgtacgcgattacatccacgtaatggatctggcggacggtcacgtcgtggcgatggaaaaactggcgaacaagccaggcgtacacatctacaacctcggcgctggcgtaggcaacagcgtgctggacgtggttaatgccttcagcaaagcctgcggcaaaccggttaattatcattttgcaccgcgtcgcgagggcgaccttccggcctactgggcggacgccagcaaagccgaccgtgaactgaactggcgcgtaacgcgcacactcgatgaaatggcgcaggacacctggcactggcagtcacgccatccacagggatatcccgattaatgactcgagtgatctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 9 - DNA, 8232 bp, synthetic construct
  misc_feature 1..8232: note = construct pCOLA-glmUM-glmS
  source 1..8232: mol_type = other DNA
cagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactataggggaattgtgagcggataacaattcccctgtagaaataattttgtttaactttaataaggagatataccatgctgaacaacgcgatgtctgttgttatcctggcggcgggtaaaggtacccgtatgtactctgacctgccgaaagttctgcacaccctggcgggtaaagcgatggttcagcacgttatcgacgcggcgaacgaactgggtgcggcgcacgttcacctggtttacggtcacggtggtgacctgctgaaacaggcgctgaaagacgacaacctgaactgggttctgcaggcggaacagctgggtaccggtcacgcgatgcagcaggcggcgccgttcttcgcggacgacgaagacatcctgatgctgtacggtgacgttccgctgatctctgttgaaaccctgcagcgtctgcgtgacgcgaaaccgcagggtggtatcggtctgctgaccgttaaactggacgacccgaccggttacggtcgtatcacccgtgaaaacggtaaagtaaccggtatcgttgaacacaaagacgcgaccgacgaacagcgtcagatccaggagatcaacaccggtatcctgatcgcgaacggtgcagacatgaaacgttggctggcgaaactgaccaacaacaacgcgcagggtgaatactacatcaccgacatcatcgcgctggcgtaccaggaaggtcgtgaaatcgttgcggttcacccgcagcgtctgtctgaagttgaaggtgttaacaaccgtctgcagctgtctcgtctggaacgtgtttaccagtctgaacaggcggaaaaactgctgctggcgggtgttatgctgcgtgacccggcgcgtttcgacctgcgtggtaccctgacccacggtcgtgacgttgaaatcgacaccaacgttatcatcgaaggtaacgttaccctgggtcaccgtgtaaaaatcggcaccggttgcgttatcaaaaactctgttatcggtgacgactgcgaaatctctccgtacaccgttgttgaagacgcgaacctggcggcggcgtgcaccatcggtccgttcgcgcgtctgcgtccgggtgcggaactgctggaaggtgcgcacgttggtaacttcgttgaaatgaaaaaagcgcgtctgggtaaaggttctaaagcgggtcacctgacctacctgggtgacgcggaaatcggtgacaacgttaacatcggtgcgggtaccatcacctgcaactacgacggtgcgaacaaattcaaaaccatcatcggtgacgacgttttcgttggttctgacacccagctggttgcgccggttaccgttggtaaaggtgcgaccatcgcggcgggtaccaccgttacccgtaacgttggtgaaaacgcgctggcgatctctcgtgttccgcagacccagaaagaaggttggcgtcgtccggttaaaaaaaaataacgaaggagatagaaccatgtccaaccgtaaatacttcggtacggacggtatccgtggtcgtgtaggtgatgctccgattacgccggatttcgtcctgaaactcggttgggcagcgggtaaagttctcgcacgtcacggctctcgtaaaatcatcatcggtaaagacacccgtatctctggttacatgctcgaatctgcactggaagcgggtctggctgcagctggtctgtctgcactgttcacgggtccgatgccaaccccagctgtagcgtacctgactcgcactttccgtgcagaagcaggtatcgtgatctctgcctctcacaacccgttctacgacaacggtatcaaattcttcagcatcgatggtaccaaactcccagacgcggttgaagaggctatcgaagcggaaatggagaaagaaatctcttgtgtagactctgccgaactcggtaaagcgtctcgtatcgttgatgcagcgggtcgttacatcgagttctgcaaagccacctttccgaacgaactgagcctgtctgagctgaaaatcgtcgtagactgtgccaacggtgcgacttaccacattgccccaaacgtactgcgtgagctgggtgctaacgtcatcgcgatcggttgtgaaccgaacggtgtcaacatcaacgcggaagtaggtgcgaccgatgttcgtgcactgcaggctcgtgtactcgcggagaaagcggatctcggtatcgcctttgacggtgatggtgaccgtgttatcatggttgaccacgaaggtaacaaagtggatggtgaccagatcatgtacatcattgcccgtgaaggtctgcgtcagggtcagctgcgtggtggtgcagtaggtaccctcatgagcaacatgggtctggaactggccctgaaacagctgggtatcccattcgctcgtgctaaagtaggcgaccgttacgttctggagaaaatgcaggagaaaggttggcgtatcggtgccgaaaactctggtcacgtcatcctgctggacaaaaccactaccggtgacggtatcgtagcaggtctgcaggtactcgccgctatggcccgtaaccacatgtccctccatgacctctgctctggtatgaaaatgttcccgcagatcctggttaacgttcgttacaccgcaggttctggtgatccgctggaacacgagtctgtgaaagccgttaccgcagaagtggaagcggccctgggtaaccgtggtcgtgtactgctgcgtaaatccggtactgagccactgatccgtgttatggttgagggcgaagatgaagcccaggtcaccgaatttgcgcaccgtattgccgacgcagtcaaagcggtttaaatgggcagcagccatcaccatcatcaccacagccaggatccgaattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgtgcggtatcgttggtgctatcgcacagcgtgatgtagcggagatcctcctggaaggtctgcgtcgtctcgaataccgtggttacgactctgccggtctggcagtagtggatgcagaaggtcacatgactcgtctgcgtcgtctgggtaaagtgcagatgctcgcgcaggcggcggaagaacacccactccacggtggtacgggtatcgcacacactcgttgggcaacccacggtgaaccgtctgaggtcaacgcacacccgcatgttagcgagcacatcgtagtcgttcacaacggtatcatcgagaaccacgaaccactccgtgaggaactcaaagcccgtggttacaccttcgtaagcgaaaccgacacggaagttatcgcccacctcgttaactgggaactcaaacagggtggtactctgcgtgaagcagttctgcgtgccattccacagctgcgtggtgcatacggtaccgtgatcatggactctcgtcatccggataccctgctcgccgcacgttctggttctccactcgttatcggtctgggtatgggtgagaacttcatcgcctctgatcagctggccctgctcccagttacccgtcgcttcatcttcctggaagagggtgacatcgccgaaatcacccgtcgttccgttaacatcttcgacaaaacgggtgcggaagttaaacgtcaggacatcgagtctaacctgcagtatgacgctggtgacaaaggcatctaccgtcactacatgcagaaagagatctacgaacagccgaacgcgatcaaaaacaccctgaccggtcgtatctctcacggtcaggttgacctgtctgagctgggtccaaacgcggacgaactcctgtccaaagtcgagcacatccagatcctggcttgtggtacctcttacaactccggtatggtttctcgttactggttcgaatctctggcaggtatcccatgcgacgttgaaatcgcctccgaattccgttatcgtaaatctgcggtacgtcgtaactccctcatgatcaccctgtctcagtctggtgaaaccgctgatactctggcaggtctgcgtctcagcaaagaactgggttacctgggttctctggccatctgcaacgttccgggttctagcctggttcgtgagtctgacctggctctgatgaccaacgcgggtacggagatcggtgttgcctctaccaaagcgttcactacccagctcactgtcctgctgatgctggttgccaaactgtctcgtctcaaaggcctcgacgctagcatcgaacacgacatcgtacacggtctgcaggccctcccatctcgtatcgagcagatgctgtctcaggacaaacgtatcgaagcactggcagaagacttcagcgacaaacaccacgcgctgtttctgggtcgtggtgaccagtacccaattgcgctggaaggtgccctgaaactgaaagagatcagctacatccatgcagaggcatacgcagcgggtgagctgaaacatggtccactggccctgatcgacgcagatatgccggttattgtggttgctccgaacaacgaactgctggagaaactgaaatccaacatcgaggaagtacgtgcgcgtggtggtcagctgtacgtgtttgctgaccaggacgcgggtttcgtttccagcgacaacatgcacatcatcgaaatgccgcatgttgaagaggtaatcgcgccaatcttctacaccgtaccgctgcagctgctggcgtaccatgtagccctgatcaaaggtacggacgttgaccagccgcgtaacctggcgaaatccgtgaccgtggaataagacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaagtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggcatgctagcgcagaaacgtcctagaagatgccaggaggatacttagcagagagacaataaggccggagcgaagccgtttttccataggctccgcccccctgacgaacatcacgaaatctgacgctcaaatcagtggtggcgaaacccgacaggactataaagataccaggcgtttccccctgatggctccctcttgcgctctcctgttcccgtcctgcggcgtccgtgttgtggtggaggctttacccaaatcaccacgtcccgttccgtgtagacagttcgctccaagctgggctgtgtgcaagaaccccccgttcagcccgactgctgcgccttatccggtaactatcatcttgagtccaacccggaaagacacgacaaaacgccactggcagcagccattggtaactgagaattagtggatttagatatcgagagtcttgaagtggtggcctaacagaggctacactgaaaggacagtatttggtatctgcgctccactaaagccagttaccaggttaagcagttccccaactgacttaaccttcgatcaaaccgcctccccaggcggttttttcgtttacagagcaggagattacgacgatcgtaaaaggatctcaagaagatcctttacggattcccgacaccatcactctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgat

SEQ ID NO: 10 - DNA, 6792 bp, synthetic construct
  misc_feature 1..6792: note = construct pINT-malE-PmgalT7
  source 1..6792: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgaagatcctttgatcttttctacggggtctgacgctcagtggaacgaaaactcacgttaagggattttggtcatgagattatcaaaaaggatcttcacctagatccttttaaactagtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccactgcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaaacgagacatactcttcctttttcaatattattgaagcatttatcagggttattgtctcatgagcggatacatatttgaatgtatttagaaaaataaacaaataggggttccgcgcacatttccccgaaaagtgccacctgaaattggccagatgattaattcctaatttttgttgacactctatcattgatagagttattttaccactccctatcagtgatagagaaaagtgaaatgaatagttcgacaaaaatctagaaataattttgtttaactttaagaaggagatatacaaatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactatgagcggtgaacactatgtcattagcctgtcgtcggcagttgaacgtcgccagcacattcgtaaccagttttcgcagaagaacatcccgtttcagtttttcgatgcaatttcaccgtcgccgctgctggaccagctggtgctgcaatttttcccgcgtctggcggatagctctctgaccggcggtgaaaaagcctgctttatgagccatctgtctctgtggcacaaatgtgtggaagaaaacctgccgtatattgtggtttttgaagatgacatcgttctgggcaaagatgcggacaagttcctgattggtgatgaatggctgttttctcgtttcgacccggaagaaatctttattatccgcctggaaaccttcctgcagaaagtcgtgtgcgaaagcacccatattgccccgtatacgcaccgcgattttctgagtctgaaatccgcacatttcggcacggctggttacgtcatcagtcagggcgcggccaaatttctgctggatattttcaagaacatctccaatgaacacattgcgccgatcgacgaactgatttttaaccagttcctggttaagaactcattcaacgtctaccaactgtcgccggcaatctgtgttcaggaactgcaactgaacaatgaaagttccgctctgcagagccaactggaactggaacgtaacaaattccgcaataaaaagtctgaagaactgaagcgtaaccgcaagaacttcatcgaaaagttcatctacatcctgaaaaagccgaagcgtatgctggataacaataagcgtaagcgcgaagagagtaagatcgaaaacgacaagatgatcatcgaatttaaatgagcggccgcgtcgacacgcaaaaaggccatccgtcaggatggccttctgcttaattatctagatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcatgtatgaatatcctccttagttcctattccgaagggtaatggcatcagggaatggcgaacgcgctccccacactaccatcatgtatgaatatcctccttagttcctattccgaagttcctattctctagaaagtataggaacttcggtggaacgacgcgtaactcacgttaagggattttggtcatgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgagtggcagggcggggcgtaaggcgcgccatttaaatgaagttcctattccgaagttcctattctctagaaagtataggaacttcgaagcagctccagcctacacaatcgctcaagacggaacccgcgcttggcaggaaagtaatagggatagcagctccagcctacacaatcgctcaagacgtgtaatgctgcacaataaccctgctgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 11 - DNA, 6702 bp, synthetic construct
  misc_feature 1..6702: note = construct pINT-malE-MsgalT8
  source 1..6702: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgaagatcctttgatcttttctacggggtctgacgctcagtggaacgaaaactcacgttaagggattttggtcatgagattatcaaaaaggatcttcacctagatccttttaaactagtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccactgcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaaacgagacatactcttcctttttcaatattattgaagcatttatcagggttattgtctcatgagcggatacatatttgaatgtatttagaaaaataaacaaataggggttccgcgcacatttccccgaaaagtgccacctgaaattggccagatgattaattcctaatttttgttgacactctatcattgatagagttattttaccactccctatcagtgatagagaaaagtgaaatgaatagttcgacaaaaatctagaaataattttgtttaactttaagaaggagatatacaaatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactatggatgaaatcaaactgtcggtggttatgccgtattacaaacgtctgcgtgaatttatgcgtgtcctgccgctgaatgcccgcttctttagccgtcatgaatatgaagtggttctgagtctggacgaaccgtccgaagaagccgatctgctgcgtgtcctgcgcgacttcccgtctattcgttggcgcgttctggtcaatgacctggatcacccgtggcgtccgccgtgccgtgcactgaacgttggcatccgtaatgctctgggtgaaaacgtcctggtcgtgagcccggaatctgcgtttgtgaccgatgttccggcacgcgctctggatcatattgcagcaaacccgggtaccgcagctctgggtcacgtttgttttgcaacgttcgatgcgctggaagcccgtcagggcagcctggaaaaaacgtgcgctccgccgtggaatctgtatggttctatctgtgtcccgcgtgaacgtctggcacgtgtgcatggctacgacgaaagcttcgatcgctggggcggtgatgacgataacctgcgtattcgcctgatgcagaccgaaacgtatctgcatccgctggacgatatgcgcatcctgcacctgagttttgaagcccgtaaagtgcgtcaagcagcagaaccgccgtccccggaatacgcagaacgtattttccagccggtgtcaccgcaagcaaatccgggcggttggggtgaatcgtttcagcgcgttgcgttcgattggcgtcgccaatgagcggccgcgtcgacacgcaaaaaggccatccgtcaggatggccttctgcttaattatctagatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcatgtatgaatatcctccttagttcctattccgaagggtaatggcatcagggaatggcgaacgcgctccccacactaccatcatgtatgaatatcctccttagttcctattccgaagttcctattctctagaaagtataggaacttcggtggaacgacgcgtaactcacgttaagggattttggtcatgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgagtggcagggcggggcgtaaggcgcgccatttaaatgaagttcctattccgaagttcctattctctagaaagtataggaacttcgaagcagctccagcctacacaatcgctcaagacggaacccgcgcttggcaggaaagtaatagggatagcagctccagcctacacaatcgctcaagacgtgtaatgctgcacaataaccctgctgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 12 - DNA, 6777 bp, synthetic construct
  misc_feature 1..6777: note = construct pINT-malE-KdgalT10
  source 1..6777: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgaagatcctttgatcttttctacggggtctgacgctcagtggaacgaaaactcacgttaagggattttggtcatgagattatcaaaaaggatcttcacctagatccttttaaactagtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccactgcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaaacgagacatactcttcctttttcaatattattgaagcatttatcagggttattgtctcatgagcggatacatatttgaatgtatttagaaaaataaacaaataggggttccgcgcacatttccccgaaaagtgccacctgaaattggccagatgattaattcctaatttttgttgacactctatcattgatagagttattttaccactccctatcagtgatagagaaaagtgaaatgaatagttcgacaaaaatctagaaataattttgtttaactttaagaaggagatatacaaatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactatggaaaactatgtcgtctctatccgcaccgcagcccaacgccgccagcatgtcgccgccgaattcaataagcaccaaatcgcctttcatttctttgatgcggtgaccccggaaacgctggcggaaagcatcgcagaacactgcccgaacctggcagacgcctttctgaccggcggtgaaaagggctgtttcatgtctcatgtctgcctgtgggcaaaatgtgtggctgatgacctgccgtatattggcatctttgaagatgacgttattttcggtcagaacagctctcgttttctgaatgataccaaatggctggacgaacgttttcagaaccaatcattcattatccgcatggaaacgtttctgaaggcgaacccggttgccctgagcaaatctggcgtccgtccgttcaatggtcgtaagatcctgcgcctgcagagttttggcttcggtaccgcggcctatctgatttcccagcaaaccgcaatcacgctgctgaattggattcgcgaagtcgctccggaaaaactggaaccgattgataacatgctgtttaatgcagcttcagaaattccggaaatccagatgtaccaaatctcgccggccctgtgcattcaggaactgcaactgaaccgcgcagatagttccctgtcatcgaccctggaagacggtcgtctggcacgtcaccagcaactggatggcggtaaaacccagccggaacagacgcaagaaaaccgtaacatcttcgcatgggctaagaacaagatcgtgaaggaatacaagcgcgttaaacgtcgctggacggatgacaaaaagattgttccgttcaaatgagcggccgcgtcgacacgcaaaaaggccatccgtcaggatggccttctgcttaattatctagatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcatgtatgaatatcctccttagttcctattccgaagggtaatggcatcagggaatggcgaacgcgctccccacactaccatcatgtatgaatatcctccttagttcctattccgaagttcctattctctagaaagtataggaacttcggtggaacgacgcgtaactcacgttaagggattttggtcatgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgagtggcagggcggggcgtaaggcgcgccatttaaatgaagttcctattccgaagttcctattctctagaaagtataggaacttcgaagcagctccagcctacacaatcgctcaagacggaacccgcgcttggcaggaaagtaatagggatagcagctccagcctacacaatcgctcaagacgtgtaatgctgcacaataaccctgctgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 13 - DNA, 6786 bp, synthetic construct
  misc_feature 1..6786: note = construct pINT-malE-gatD
  source 1..6786: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgaagatcctttgatcttttctacggggtctgacgctcagtggaacgaaaactcacgttaagggattttggtcatgagattatcaaaaaggatcttcacctagatccttttaaactagtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccactgcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaaacgagacatactcttcctttttcaatattattgaagcatttatcagggttattgtctcatgagcggatacatatttgaatgtatttagaaaaataaacaaataggggttccgcgcacatttccccgaaaagtgccacctgaaattggccagatgattaattcctaatttttgttgacactctatcattgatagagttattttaccactccctatcagtgatagagaaaagtgaaatgaatagttcgacaaaaatctagaaataattttgtttaactttaagaaggagatatacaaatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactatgtcctcagctttccattacgtcattagcctggcatcggcagttgaacgccgtcagcacattagcgaacagttttcccaatacgacattccgtttcagtttttcgatgcgatcagtccgtccccgctgctgaaccagctggtgtctcaatttttcccgtccctggccgatagctctctgaccgacggcgaaaaaggttgctttatttcacatctgtcgctgtggcacaagtgtgttgaaaagaacctgccgtatattgtggtttttgaagatgacatcctgctgggcaagaatgcagataaattcctgattgaagacgaatggtttttctctcgttttaacacgaatgatgtcttcatcgtgcgcctggaaacctttctgcagaaagtgtattgccaaccgagctacatcaagtcttactacaaccgtgaactgctgaccctgaaaagcacgcatttcggcaccgcaggttatattatcagtctgggtgcggccaagtttctgctgtccctgttcaacaaaatgcacattgaagaagttgctccgatcgatgaactgctgtttaataagttcctggaacgcaaagactttacggtctaccagttcagtccggcactgtgcattcaggaactgcaactgaacaaatcagatgctgtcctgctgtcgcaactggaactggaacgtagcaaatgtcgcattatgaccgaatctcgtatcggccgcgaaaagaaaaaactgaaggataagatcatccatgttctgacgaagccgaaacgtatgctggaaaagaaacgtcagcgcaatgaagacaagaaaatcaccatgattatcgaatttgaatgagcggccgcgtcgacacgcaaaaaggccatccgtcaggatggccttctgcttaattatctagatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcatgtatgaatatcctccttagttcctattccgaagggtaatggcatcagggaatggcgaacgcgctccccacactaccatcatgtatgaatatcctccttagttcctattccgaagttcctattctctagaaagtataggaacttcggtggaacgacgcgtaactcacgttaagggattttggtcatgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgagtggcagggcggggcgtaaggcgcgccatttaaatgaagttcctattccgaagttcctattctctagaaagtataggaacttcgaagcagctccagcctacacaatcgctcaagacggaacccgcgcttggcaggaaagtaatagggatagcagctccagcctacacaatcgctcaagacgtgtaatgctgcacaataaccctgctgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 14 - DNA, 7506 bp, synthetic construct
  misc_feature 1..7506: note = construct pINT-malE-BFgalT2
  source 1..7506: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgaagatcctttgatcttttctacggggtctgacgctcagtggaacgaaaactcacgttaagggattttggtcatgagattatcaaaaaggatcttcacctagatccttttaaactagtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccactgcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaaacgagacatactcttcctttttcaatattattgaagcatttatcagggttattgtctcatgagcggatacatatttgaatgtatttagaaaaataaacaaataggggttccgcgcacatttccccgaaaagtgccacctgaaattggccagatgattaattcctaatttttgttgacactctatcattgatagagttattttaccactccctatcagtgatagagaaaagtgaaatgaatagttcgacaaaaatctagaaataattttgtttaactttaagaaggagatatacaaatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactatgaacgtgaataagccgaccaccgaaaagaaactgattgacctgaacaacgacattatccataactttgatgtgagcattgtgatgagcttctataagcgttacaccgaatttcgcaaagtgctgccgcataacgcgccgtatctgcagcgtaatggcattgaagtcattatcgtgctggatgacccggatgaaaaaagcgaactgctgatgctgctgcaaaactatccgttcatcaattggaagctgattatcaacgaacgtaaacatgcaccgcgcaaccacgcttctgttctgaatgtcggtctgaaacatgcgaccaaaaagtatattctgcagatcgatccggaagttgaatttctgacggatattatctggcaaatgcgtgacgccattgaaaaatatccgatgcactacatcctggcgatgatggcctatgtcccgtacgaacaggaactgaccgaaaacaacatcaaggaactggatttcatcccgtggggcaacctgatggtggaacgcaatcatctgtataaactgcacggttacgatgaaaccttcattacgtggggcggtgaagataacaatatgcgtgcgcgcctggacatgtcaggcattaaaaagtttatcctgccggaagccaagaccatccatcgtgaaaagaactatgatccgaatgaacgttcgaagcgcattaataaacacagtatctccgactggcgcaaaatgaactacccgtcagaagcaattgctaataaggatatctggggctcggaattcaacaaagttatttatgattggcaggacaatcaatacgccaaagatctgtgctatacctacctgcagcaatttattggtttcgaaatccgtcatccggcggcctttcgtaaacgccacaaaaagattgtcctgtgtcaggcatataacgaagaaaaactgatcgaaggcttcctgacgaacatggctaattactttgatggtattatcctgctggatgacgaaagtaccgatcgcacgtgggacctggcaatccatgataagatcatcctgaaggtgaaaaagaaacgttccggttttaatgatctggaaaaccgcaatattctgctggacctgtcagcgtttttccagtcggaatggttttgcttcatggatatcgacgaacgtttcgatgaacgctttaccaacttcagcgaattcgaaaacaacaaggaaatccacgtggtttcttttcgtggcgtgtatctgtggaatgatgaacagagctacaagggcgacattccgaactctaataaaggtatcctgaccgtttatcgtatgttccgcccgattggtcatacccacatcaacacgcataagaaactgcacttcattgcgacgccgtattttaccaacacgtggcagagtaatatcctgtttaaggattacggctccatgaaagaaaatgaccgtattcgcaagtatgaacgctacatccaggaagatcagcaaaaagacatgagctctggttatgattacctgctgaacagcgaaaatctgtatcaactggacaaaattgaagaatactgagcggccgcgtcgacacgcaaaaaggccatccgtcaggatggccttctgcttaattatctagatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcatgtatgaatatcctccttagttcctattccgaagggtaatggcatcagggaatggcgaacgcgctccccacactaccatcatgtatgaatatcctccttagttcctattccgaagttcctattctctagaaagtataggaacttcggtggaacgacgcgtaactcacgttaagggattttggtcatgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgagtggcagggcggggcgtaaggcgcgccatttaaatgaagttcctattccgaagttcctattctctagaaagtataggaacttcgaagcagctccagcctacacaatcgctcaagacggaacccgcgcttggcaggaaagtaatagggatagcagctccagcctacacaatcgctcaagacgtgtaatgctgcacaataaccctgctgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 15 - DNA, 6717 bp, synthetic construct
  misc_feature 1..6717: note = construct pINT-malE-lsgD
  source 1..6717: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgaagatcctttgatcttttctacggggtctgacgctcagtggaacgaaaactcacgttaagggattttggtcatgagattatcaaaaaggatcttcacctagatccttttaaactagtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccactgcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaaacgagacatactcttcctttttcaatattattgaagcatttatcagggttattgtctcatgagcggatacatatttgaatgtatttagaaaaataaacaaataggggttccgcgcacatttccccgaaaagtgccacctgaaattggccagatgattaattcctaatttttgttgacactctatcattgatagagttattttaccactccctatcagtgatagagaaaagtgaaatgaatagttcgacaaaaatctagaaataattttgtttaactttaagaaggagatatacaaatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactatgctgaagaagtacctgattagcctggataaggacattcaacgccgcaagctgtttttctcgcagaagaacacggaagattttcaaattttctcagcgatcaacaccatgcagaaagattgggacgaactggcatcgatcttcaacatcgaacaattcaaggctcattacttccgtaacgtcaccaagggcgaaattggttgcacgctgagtcacctgtccgtctatcagaaaattgtggaagataacgacatcgcagaagattcatacgctctggtttgtgaagatgacgccctgtttcatctggatttccagcaaaatctgaccgcactgctgagtgaaaaactggaagctgaaattatcctgctgggccagtccaacattaacaattttaatgatacggacctggaaatcaattacccgaccacgtttagcttcctgtgcaaaaagaccggtaacgtgaattatgcgttcccgtataaatcttactttgccggcacggttggttacctgattaaaaagagcgcggcccgtcgcttcattcagcaaatctctcagaacaaaccgttttggctggcggatgactttctgctgttcgaacaaaacttcaatatccgtaataaggtggttcgcccgctgatggttattgaaaacccggtcctgatctcaaatctggaatcggtgcgcggcagcctgtctaacaatctgctgaaaaagctgatgaaatatccgctgaaaaagatttttgcgatcaaaaagaacctggccaattaagcggccgcgtcgacacgcaaaaaggccatccgtcaggatggccttctgcttaattatctagatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcatgtatgaatatcctccttagttcctattccgaagggtaatggcatcagggaatggcgaacgcgctccccacactaccatcatgtatgaatatcctccttagttcctattccgaagttcctattctctagaaagtataggaacttcggtggaacgacgcgtaactcacgttaagggattttggtcatgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgagtggcagggcggggcgtaaggcgcgccatttaaatgaagttcctattccgaagttcctattctctagaaagtataggaacttcgaagcagctccagcctacacaatcgctcaagacggaacccgcgcttggcaggaaagtaatagggatagcagctccagcctacacaatcgctcaagacgtgtaatgctgcacaataaccctgctgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 16 - DNA, 6769 bp, synthetic construct
  misc_feature 1..6769: note = construct pINT-malE-HPgalT
  source 1..6769: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgaagatcctttgatcttttctacggggtctgacgctcagtggaacgaaaactcacgttaagggattttggtcatgagattatcaaaaaggatcttcacctagatccttttaaactagtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccactgcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaaacgagacatactcttcctttttcaatattattgaagcatttatcagggttattgtctcatgagcggatacatatttgaatgtatttagaaaaataaacaaataggggttccgcgcacatttccccgaaaagtgccacctgaaattggccagatgattaattcctaatttttgttgacactctatcattgatagagttattttaccactccctatcagtgatagagaaaagtgaaatgaatagttcgacaaaaatctagaaataattttgtttaactttaagaaggagatatacaaatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactatgcgtgtgtttattatttccctgaatcaaaaagtgtgtgataccttcggtctggtgttccgtgatacgacgaccctgctgaacaacattaacgcgacccatcaccaggcccaaatttttgatgcaatctactccaaaacgttcgaaggcggtctgcatccgctggttaaaaaacatctgcacccgtactttattacccagaacatcaaagacatgggcattaccacgaatctgatcagcgaagtctctaaattctactacgctctgaaataccatgcgaaattcatgagcctgggcgaactgggttgctatgctagtcactactccctgtgggaaaaatgcattgaactgaacgaagcgatttgtatcctggaagatgacatcacgctgaaagaagattttaaagaaggcctggacttcctggaaaaacatattcaggaactgggttatgtgcgtctgatgcacctgctgtacgatccgaatgttaaaagcgaaccgctgaaccataaaaatcacgaaatccaggaacgcgtgggcattatcaaagcctattctcatggcgttggcacccaaggttacgtcattacgccgaaaatcgcaaaagtcttcaaaaaacatagtcgtaaatgggtggttccggtggataccattatggacgcgacgtttatccacggtgtcaaaaatctggtgctgcaaccgttcgttattgccgatgacgaacaaatttcaaccatcgcacgcaaagaagaaccgtattcgccgaaaatcgccctgatgcgtgaactgcacttcaaatacctgaaatactggcaattcgtctaagcggccgcgtcgacacgcaaaaaggccatccatccgtcaggatggccttctgcttaattatctagatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcatgtatgaatatcctccttagttcctattccgaagggtaatggcatcagggaatggcgaacgcgctccccacactaccatcatgtatgaatatcctccttagttcctattccgaagttcctattctctagaaagtataggaacttcggtggaacgacgcgtaactcacgttaagggattttggtcatgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgagtggcagggcggggcgtaaggcgcgccatttaaatgaagttcctattccgaagttcctattctctagaaagtataggaacttcgaagcagctccagcctacacaatcgctcaagacggaacccgcgcttggcaggaaagtaatagggatagcagctccagcctacacaatcgctcaagacgtgtaatgctgcacaataaccctgctgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 17 - DNA, 4714 bp, synthetic construct
  misc_feature 1..4714: note = construct pACYC-waaX
  source 1..4714: mol_type = other DNA
ggggaattgtgagcggataacaattcccctgtagaaataattttgtttaactttaataaggagatataccatgaaagtgtttgtggtcaacctggataaggataaggataaaaaagaaaaaatcaagaatgaatgccgcaacgcagaactggactatgaaattatctcagcagttgatggccgtgaactgagctacaacgaactgaaatctaaggtccatccggtgtcactgaattatctgtcgaaaggcgaaattggttgcgtcctgtcccaccagcgtatttacaaacgcatcctggatgacgatattgactatgctctgatcctggaagacgatgtggaactgagtcaagatatcaaggtttttctgaaggaattcctgtccgtcaaagacaagaacaaaggcgatgtgtttctgctgtacccgtcaggtctgcgtttcctgaaccgtcgcatcaacgtgtcgcatgattatttcttttatgaagcgtacaacagctcttgtgcccacggttatattatcagcaacaaagcggccaaaaagctgattcgcatcaataccccgattatcctggttgcagatgcttggctgtggttttaccagatttctctgctgaaagtgtatgttctgaacaaagaactggttcgtgcatatgacgtcgataaaagtctgtccaccatcgaaacggaacgcagcctgctgctggacgaaaaggaaaagcatcagatgcaaatcatcaaaaagcaaccgctgtactacctgatcaagtactaccacaagtacatccgtcgcctgttcatcaataaggataaataagaattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 18 - DNA, 4760 bp, synthetic construct
  misc_feature 1..4760: note = construct pACYC-wbdO
  source 1..4760: mol_type = other DNA
ggggaattgtgagcggataacaattcccctgtagaaataattttgtttaactttaataaggagatataccatgctgacggaagtgcgcccggtctctacgacgaaaccgctggtgtctgtgattctgccggtgaacaaattcaacccgtatctggatcgtgcaattcattcaatcctgagtcagtcctatccgtcgattgaactgattatcattgcaaacaattgcaccaatgactttttcgatgctctgaaaaaacgtgaatgtgaaaccattaaagtgctgcgcacgaacatcgcgtatctgccgtactgcctgaataaaggcctggatctgtgtaacggtgactttgttgcccgcatggattcagatgacatttcgcacccggaacgtatcgatcgccaggtcgacttcctgattaacaatccggacatcgatgtggttggcaccaatgcagtctatattgatgaagatgacatcgaactggaaaaaagcaacctgccggtgaacaataacgctattcgtaaaatgctgccgtataaatgctgtctggtgcatccgtctgttatgtttcgcaaaaatgtcgtgatcaccagcggcggttacatgttcgcgaattattctgaagattacgaactgtggaaccgtctggccgttgaaggccgcaatttttataacctgagcgaatacctgctgtattaccgtctgcacaataaccaatcaacgtcgaaaaataacctgtttatggtgatggcgaacgatgtcgccattaaagtgaaatatttcctgctgaccaagaaaattagctacctgctgggtatcattcgcacggtcttttctgtgttctattgcaaatacatcaaatgaattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 19 - DNA, 4795 bp, synthetic construct
  misc_feature 1..4795: note = construct pACYC-furA
  source 1..4795: mol_type = other DNA
ggggaattgtgagcggataacaattcccctgtagaaataattttgtttaactttaataaggagatataccatggataaaatcaaacagggcagcgcctctctggttgtcggtgaccagcaagaaaaacatccggtggtttcagtgctgctgccggttaatcgtgtcgatcgctttttcattccggcagttgaatcgatcctgacccaaacgctgcaggattttgaactgatcattatcgctaatggctgtagcaccgaacatctgaacaaaattcgtctgacgtatggtgatcacaatcgtgttcgcattctgaacaccgaaatcaaaggcctgccgtttgcgctgaatctgggcgtgcacaacgcccgtggtctgtatattgcacgcatggatgctgatgacatttctatcccggaacgcctggaaaaacaactgaatacgctggaacagaacaagaaaattggcgtcgtgagctctggtgtggactttattgatgaaaatgaccaggcgatccgtgagggtaaattcccggaactgaccgacaaagatcatcgtcgcctgctgccgctgatttgctgtatcgcccacccgacggttatggtccgcaaagaaattatcaacaaactgggcggttatagttttggtagtttctccgaagactacgatctgtggctgcgtattatgcgcgaactgccggaagttgaattttatcgtatcccggaatccctgctgaaataccgtcgccatggcaatcaggccaccagttccaaaaacattaagaaaattcgcgcgtacaactcagccctgaaaattcgtgaactgtttctgtcgcgcaaactgaaattcattatcggtattatcctgccggcacgtatggtgaccctgtggcgcaaatgagaattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 20 - DNA, 6383 bp, synthetic construct
  misc_feature 1..6383: note = construct pET-PmnagT
  source 1..6383: mol_type = other DNA
ggggaattgtgagcggataacaattcccctctagaaataattttgtttaactttaagaaggagatataccatgggcagcagccatcaccatcatcaccacagccaggatccgaattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggaaaataaacctttagtttcagttttgatttgtgcttataatgtcgagaaatatattgaagaatgtattaatgcagtgattaatcagacatataagaacttagaaattattattgtgaatgatggttcttctgataatacttattttcttttaaaaaagttagctgaaaaagataatcgtataaaaatattaaatttcaataatcatattggaataatttctgctttaaatgaaggtttaaaagagatagctggagaatatattgctcgaacagattctgatgatataactaagccagattggattgagaaaatattaacttgtatgcaaaatgatcctaaaatcatcgctatgggatcttatcttactgtcttgtcagaagaaaataatggtagtgtgcttgctaatcatcataaaaataaagttgaatggaaaaatccattagagcacaaagatattgttgagaaaatgttatttggtaatcctattcataataattcaatggttatgagaagtgagatatatacaaagtatcacttaatttatgatccagattatcattatgctgaagattataaattttggctggaagttagtcgaattgggaaattagcaaattatcctgagtcactcgtatattatagacttcaccgaaatcaaacatcttctattcataatagccaacaagaaataaatggtaaaaaattacgtttacaagctcttaattattatttaaaagatcttggtattgattatcagttacctgaaaaatttttattcaaagatatagcgttattgcaagaaatattttatgaacgaggtatgtttagagaaaatataataaggcgtatcatctacgaatgttatctttccttgggagagtataattataaagatatttattattttttaataaataaaaataactttctttctataaaagacaaatttaaaataataaaaaaatatcttcgtcctgataaatattcatctacttattaggacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 21 - DNA, 6711 bp, synthetic construct
  misc_feature 1..6711: note = construct pINT-yjhB
  source 1..6711: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgaagatcctttgatcttttctacggggtctgacgctcagtggaacgaaaactcacgttaagggattttggtcatgagattatcaaaaaggatcttcacctagatccttttaaactagtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccactgcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaaacgagacatactcttcctttttcaatattattgaagcatttatcagggttattgtctcatgagcggatacatatttgaatgtatttagaaaaataaacaaataggggttccgcgcacatttccccgaaaagtgccacctgaaattggccagatgattaattcctaatttttgttgacactctatcattgatagagttattttaccactccctatcagtgatagagaaaagtgaaatgaatagttcgacaaaaatctagaaataattttgtttaactttaagaaggagatatacaagagctcgagtcgaaggagatagaaccatggcaacagcatggtataaacaagttaatccaccacaacggaaagctcttttttccgcatggcttggatatgtatttgatggctttgattttatgatgatattttacattcttcatattataaaagcagatcttggcattacggatattcaggctactttaatagggacagtggccttcatagccagacctattggaggtggtttttttggtgccatggctgataaatatggtcgtaagccaatgatgatgtgggcaattttcatttactcagtcggaacaggccttagcggtattgctacaaacttatatatgctcgcagtttgccgttttattgttggcttagggatgtctggtgaatatgcatgtgcttcaacttatgcggtagaaagttggcctaaaaatcttcaatctaaagctagtgcttttttggtaagtggtttttctgttggaaatattattgcggcacaaataatccctcagtttgctgaagtatatggatggagaaactctttttttataggcctgttaccagttttactagttctttggatcagaaaaagtgctccagaaagtcaggagtggattgaagataaatataaggataaatcaacatttttgtctgtcttcagaaaaccacatctttcaatctctatgatcgttttcctcgtctgtttttgtctatttggtgcaaactggccgataaacggactacttccttcctacctggcagataatggagttaatacagtggtcatttcaactctgatgacaatagcaggtttaggaacactgacaggtacaatattttttggttttgttggtgataagattggtgtaaaaaaagcctttgtagtcggtctaataacttcatttattttcctttgtcctcttttttttatttctgtgaaaaactcttctcttataggattatgtctctttggattaatgtttacaaatttaggtattgcagggttggttccaaaatttatatatgattactttccaacaaaattaagaggattagggaccggtcttatttataacttaggggcaactggaggaatggccgcacctgtattagctacatacatttcaggatattatggcttaggtgtttcattattcattgttacggttgcattctctgccttattaattttgttagttggttttgatattccaggtaaaatttataaactatccgtggctaaatgataaatcgatactagcataaccccttggggcctctaaacgcgtcgacacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcatgtatgaatatcctccttagttcctattccgaagttcctattctctagaaagtataggaacttcggcgcgtcctacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagatgcttaatgaatacaacagtactgcgatgagtggcagggcggggcgtaaggcgcgccatttaaatgaagttcctattccgaagttcctattctctagaaagtataggaacttcgaagcagctccagcctacacaatcgctcaagacgtgtaatgctgcaatctgcatgcaagcttggcactggccacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggggggccatcgatgcaggtggcacttttcggggaaatgtgcgcggaacccctatttgtttatttttctaaatacattcaaatatgtatccgctcatgagacaataaccctgctgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 22 - DNA, 6867 bp, synthetic construct
  misc_feature 1..6867: note = construct pINT-yebQ
  source 1..6867: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgaagatcctttgatcttttctacggggtctgacgctcagtggaacgaaaactcacgttaagggattttggtcatgagattatcaaaaaggatcttcacctagatccttttaaactagtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccactgcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaaacgagacatactcttcctttttcaatattattgaagcatttatcagggttattgtctcatgagcggatacatatttgaatgtatttagaaaaataaacaaataggggttccgcgcacatttccccgaaaagtgccacctgaaattggccagatgattaattcctaatttttgttgacactctatcattgatagagttattttaccactccctatcagtgatagagaaaagtgaaatgaatagttcgacaaaaatctagaaataattttgtttaactttaagaaggagatatacaagagctcgagtcgaaggagatagaaccatgccaaaagttcaggccgacggcctgccattgccccagcgatacggtgcgatattaaccattgtgattggtatttcgatggccgtccttgacggcgcaatcgccaacgtcgccctgccaacaatcgccacggaccttcatgccacgccagccagttccatctgggtagtgaacgcctatcaaatcgccattgtcatctccctgctctcgttttcgtttctgggcgatatgtttggctatcgacgtatttataaatgcggtctggtcgtttttctgttgtcttcactgttctgcgccctttctgattcgctgcaaatgctcacccttgcgcgtgtcatacaaggtttcggcggtgcagcgttgatgagcgttaataccgcacttatccgcctgatctatccacaacgttttctgggtagagggatgggcataaactcgtttattgttgccgtctcttctgctgccgggccgacaattgctgcagcaatcctctccatcgcatcctggaaatggttatttttaatcaacgtaccgttaggtattatcgccctgcttctggcgatgcgttttctgccacccaatggttctcgcgccagtaaaccccgtttcgacctgcccagcgccgtgatgaacgcgttaaccttcggcctgcttatcactgcgttgagtggtttcgctcaggggcaatcgctgacgttaattgctgcggaactggtggtaatggttgttgttggtattttctttattcgccgccagctttctcttcccgtaccgctgctaccggtggatttactgcgtatcccgctgttttcactttctatttgcacatctgtttgctctttctgcgcacaaatgctggcaatggtttccctgcccttttacctgcaaaccgtgctcgggcgtagtgaagtcgaaacaggtttacttctgacaccgtggccgttagcaacgatggtgatggctccgctggcaggctatttgattgaacgcgtacatgcaggattgctgggggctttagggttgttcatcatggctgcggggcttttttccctggttctgctgcccgcgtcacctgcggatatcaatattatctggccgatgatcttatgtggtgctggatttggcttattccagtcacccaataaccacaccattattacctccgcgcctcgcgaacgtagcggtggagccagtggcatgttaggaacggctcgtctactgggtcagagtagcggcgcggcgctggtggcgctgatgctaaatcagtttggagataatggtacacacgtctcgctgatggctgcggctattctggcagtgattgctgcctgtgtcagtggtttacgtatcactcagccacgatccagggcataataaatcgatactagcataaccccttggggcctctaaacgcgtcgacacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcatgtatgaatatcctccttagttcctattccgaagttcctattctctagaaagtataggaacttcggcgcgtcctacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagatgcttaatgaatacaacagtactgcgatgagtggcagggcggggcgtaaggcgcgccatttaaatgaagttcctattccgaagttcctattctctagaaagtataggaacttcgaagcagctccagcctacacaatcgctcaagacgtgtaatgctgcaatctgcatgcaagcttggcactggccacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggggggccatcgatgcaggtggcacttttcggggaaatgtgcgcggaacccctatttgtttatttttctaaatacattcaaatatgtatccgctcatgagacaataaccctgctgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 23 - DNA, 6768 bp, synthetic construct
  misc_feature 1..6768: note = construct pINT-proP
  source 1..6768: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgaagatcctttgatcttttctacggggtctgacgctcagtggaacgaaaactcacgttaagggattttggtcatgagattatcaaaaaggatcttcacctagatccttttaaactagtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccactgcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaaacgagacatactcttcctttttcaatattattgaagcatttatcagggttattgtctcatgagcggatacatatttgaatgtatttagaaaaataaacaaataggggttccgcgcacatttccccgaaaagtgccacctgaaattggccagatgattaattcctaatttttgttgacactctatcattgatagagttattttaccactccctatcagtgatagagaaaagtgaaatgaatagttcgacaaaaatctagaaataattttgtttaactttaagaaggagatatacaagagctcgagtcgaaggagatagaaccatgccgaacaaagctgaaacctccccggcgaaactgcgtctgaaagccttcctgaaacgtatcaagattatgaacaccaccgaaaacagcaaacagaagccggttaacgtggttgcatttgctttcctgctgaccgcgtttctgacgggtatcgccagctctttccaaaccccgacgctgagcctgtttctggcgcaggaaattcaagtctctccgtttatggtgggcatgttctatacctcaaatgcagtgctgggcatcgttctgtcgcagattctggctaaatacagtgattcccaagatgaccgtcgcaagattatcattttctgcagtctgctggcgatcggcggttgtatcaccttcgcctacaaccgtaactactacgtgctgatgtttttcgcgacgttcctgctgtccctgggtagttccgcaaacccgcaggcatttgcactggcacgtgaatatgcagactacaccaaacgcgaagctatcatgtttaccacgattatgcgcacgcagatcagcctggcatggattgttggcccgccgctgtcattctcgattgcgctgggctggggttttgaatatatgtacatggtcgcggcctcagcatttctgctgtgcgctatcattgctaaagcgctgctgccgtatgtgccgcgtaaagccgtcgtgccgctgaccaagccggatgaagttgcgggtctgccggccaaaaataaaaagcagagtgacaagcaatccatccgcctgctgtttattacgtgcttcctgatgtggagttgtaacggcatgtatctgatctccatgccgctgcatgttattaatgaactgcacctgagtgaacgtctggcgggcattctgatgggtaccgcagctggcctggaaatcccggtgatgctgattgccggctatctgaccaaatacctgacgaaaaagtctctgatcctgaccgccctgttcatgggtctgtttttctatattggcatgctgtttgcagaacagacgtggcaactggtcgccctgcaggcatttaacgctatcttcattggtatcattgcgaccctgggcatggtgtactttcaagatctgatgccgggcaaaatgggttcagccaccacgctgttctcgaacgcggccaaatcatcgtggatcgttgcaggtccgtttgtcggcatcattgctcagatttggaattatagctctgtgttctacatcagcattgttctggtcgcggtgtctctgtttagcatgtctaaagttaagagcgtctaataaatcgatactagcataaccccttggggcctctaaacgcgtcgacacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcatgtatgaatatcctccttagttcctattccgaagttcctattctctagaaagtataggaacttcggcgcgtcctacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagatgcttaatgaatacaacagtactgcgatgagtggcagggcggggcgtaaggcgcgccatttaaatgaagttcctattccgaagttcctattctctagaaagtataggaacttcgaagcagctccagcctacacaatcgctcaagacgtgtaatgctgcaatctgcatgcaagcttggcactggccacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggggggccatcgatgcaggtggcacttttcggggaaatgtgcgcggaacccctatttgtttatttttctaaatacattcaaatatgtatccgctcatgagacaataaccctgctgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 24 - DNA, 6672 bp, synthetic construct
  misc_feature 1..6672: note = construct pINT-Cn-setA
  source 1..6672: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgaagatcctttgatcttttctacggggtctgacgctcagtggaacgaaaactcacgttaagggattttggtcatgagattatcaaaaaggatcttcacctagatccttttaaactagtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccactgcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaaacgagacatactcttcctttttcaatattattgaagcatttatcagggttattgtctcatgagcggatacatatttgaatgtatttagaaaaataaacaaataggggttccgcgcacatttccccgaaaagtgccacctgaaattggccagatgattaattcctaatttttgttgacactctatcattgatagagttattttaccactccctatcagtgatagagaaaagtgaaatgaatagttcgacaaaaatctagaaataattttgtttaactttaagaaggagatatacaagagctcgagtcgaaggagatagaaccatgctgtggtttctgacccgtgctcgtcgcttcaatccggtttatgcggcctttatggccgtcagcttcatgattggtgtggccggtgcactgcaggcaccgaccctgtctctgtttctgacgcgtgaagttgaagtccgcccgttttgggttggtctgttctacacggtcaacgcaattgctggcatcggtgtgagtctgctgctggccaaacgtagtgattcccaaggcgaccgtcgcaaactgattatggtgtgctgtgttatggcggtcgccaactgcgtcctgtttgcattcaatcgccattatctgaccctgatcacgctgggtgtgatgtttgcaagcattgctaataccgcgatgccgcagatcttcgcactggctcgtgaatacgccgatcgttctgcacgcgaagtggttatgtttagctctattatgcgcgcccaactgagtctggcatgggttattggcccgccgctgtccttcatgctggccctgaaatatggttttaccacgatgttcctgattgcagctggcatttttgtgatctcactggctctgattatcttcgcgctgccgtcggtgccgcgtgttgaacagccggccgaagtggcaattacccaagttagcggttggaaagattctaacgttcgcatgctgtttatcgcctcaatgctgatgtggacctgtaatacgatgtatattatcgacatgccgctgtggatttcgcaggatctgggtctgccggatgaactggccggtctgctgatgggtaccgccgcaggcattgaaatcccggctatgatcctggcgggttattacgtgaaacgttttggcaaacgcaacatgatggtcgcagctgtggcggccggtattctgttttacgttggcctgatcctgttccatagcaaaacggcgctggtcgtgctgcagctgtttaatgccgtcttcattggtattatcgcaggcatcggtatgctgtggtttcaagatctgatgccgggtcgtccgggtagcgcaaccaccctgttcaccaactcaatttcgacgggcgtgattctggccggtattctgcagggtgccctggcagaaggttttggtcactatagtgtgtactggctgatggcagctctggctgttatcgcgctgttcctgaccagccgcgttaaaaacgtctaataaatcgatactagcataaccccttggggcctctaaacgcgtcgacacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcatgtatgaatatcctccttagttcctattccgaagttcctattctctagaaagtataggaacttcggcgcgtcctacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagatgcttaatgaatacaacagtactgcgatgagtggcagggcggggcgtaaggcgcgccatttaaatgaagttcctattccgaagttcctattctctagaaagtataggaacttcgaagcagctccagcctacacaatcgctcaagacgtgtaatgctgcaatctgcatgcaagcttggcactggccacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggggggccatcgatgcaggtggcacttttcggggaaatgtgcgcggaacccctatttgtttatttttctaaatacattcaaatatgtatccgctcatgagacaataaccctgctgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 25 - DNA, 7074 bp, synthetic construct
  misc_feature 1..7074: note = construct pINT-spoVB
  source 1..7074: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgaagatcctttgatcttttctacggggtctgacgctcagtggaacgaaaactcacgttaagggattttggtcatgagattatcaaaaaggatcttcacctagatccttttaaactagtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccactgcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaaacgagacatactcttcctttttcaatattattgaagcatttatcagggttattgtctcatgagcggatacatatttgaatgtatttagaaaaataaacaaataggggttccgcgcacatttccccgaaaagtgccacctgaaattggccagatgattaattcctaatttttgttgacactctatcattgatagagttattttaccactccctatcagtgatagagaaaagtgaaatgaatagttcgacaaaaatctagaaataattttgtttaactttaagaaggagatatacaagagctcgagtcgaaggagatagaaccatgaatgactcggtcggtgctaagcgtcaaagtgcgtggaagggtgcgttcgtcctggtcgttgcgggcatcgttaccaagatcctgtctgccgtgtatcgtgttccgtttcagaacattgtcggcgatgtgggtttctatatctaccagcaagtttacccgtttctgggcattgcggtcatgctgagtacctccggttttccggtgatcatctcgaagctgatgaacgattacagcgaccataaacagaagattatgaagatcagtgcactgtatgtgacggcagcaggtctggttctgtttgccctgatgtacgcaggtgcagctccgctggcgggcttcatgggtgatgaccgtctggtcatgctgattcgcgtggcggcctttgctttcatcctgtttccgttcaccgcggtttttcgcggctatttccagggtgtgcacgacatgatgccgtctgctctgagtcagattacggaacaactgctgcgtgtggcagttctgctgggcctgtctttttggctgctgaaatccggtcgttcactgtacgcagctggtgcaggtgcagcatcaggttcgattgcaggtagtctggcagctctgtgcgttctggcagtcttctggtataaacgtgaagaaaccaaaaaggatggcggtcatatcgaaacggcggttattatcaaaaagctgctgctgtactccgtgaccatttgtatcagctctgttctgatgctgctgctgcagctggttgatgcgctgaacctgtattcgctgctgagcgacggcaccgaatcacatgcggccaaacaactgaagggcatttacgaccgtggtcagccgctgctgcaactgggtacggtgtttgcggtttccattgcagcttcactggtcccgagcatctctaaagccgtgcacgaaaataagccgttcattatcaaagaaaaggctacctctgcggtcaaactgtgcctggcggtgggcattggtgctagtgcgggcctgttttgtattctggaaccggttaacatcatgctgttccagaattccgaaggtacccagacgctgcaaatctttagtctgtccattttctttgcctcaatcgcactgaccgcagcagcaatcctgcaaggtgcaggtcatacggtgttcccggcagtcagcgtgctggctggcggtgcgctgaaatgggtcctgaacgtgtggctggttccgggttggggtattaccggtgctgcactggctacggttctggcatttgcagcagtcgcatgcctgaacctgcgtcgcatctggtcgaaaggttggctgaccaatattggcggtgtgatcgcacgtctgtgctggtgtagcctgctgatggtgtttttcctgctggtctatatgaaactgtggcagctgtttgttccggtcagccgtgccggcgcagtttgcgaatcactgtcggccagcgtgattggcggtctgctgttcatctactgtatgatccgcatgaagatcttcaccgatgaagaactgagcggcctgccgttcggttctgcgctgagtaaactgaaaaagcgtcgcgaaaagcacggtcgctaataaatcgatactagcataaccccttggggcctctaaacgcgtcgacacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcatgtatgaatatcctccttagttcctattccgaagttcctattctctagaaagtataggaacttcggcgcgtcctacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagatgcttaatgaatacaacagtactgcgatgagtggcagggcggggcgtaaggcgcgccatttaaatgaagttcctattccgaagttcctattctctagaaagtataggaacttcgaagcagctccagcctacacaatcgctcaagacgtgtaatgctgcaatctgcatgcaagcttggcactggccacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggggggccatcgatgcaggtggcacttttcggggaaatgtgcgcggaacccctatttgtttatttttctaaatacattcaaatatgtatccgctcatgagacaataaccctgctgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 26 - DNA, 6699 bp, synthetic construct
  misc_feature 1..6699: note = construct pINT-yabM
  source 1..6699: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgaagatcctttgatcttttctacggggtctgacgctcagtggaacgaaaactcacgttaagggattttggtcatgagattatcaaaaaggatcttcacctagatccttttaaactagtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccactgcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaaacgagacatactcttcctttttcaatattattgaagcatttatcagggttattgtctcatgagcggatacatatttgaatgtatttagaaaaataaacaaataggggttccgcgcacatttccccgaaaagtgccacctgaaattggccagatgattaattcctaatttttgttgacactctatcattgatagagttattttaccactccctatcagtgatagagaaaagtgaaatgaatagttcgacaaaaatctagaaataattttgtttaactttaagaaggagatatacaagagctcgagtcgaaggagatagaaccatgaaggctctgtggtcgcgtcgccgtcgtatccacccggtctatctggcttttatggcagtttcgtttatggttggcatcgcaggtgcgctgcagtcaccgaccctgtcgctgtttctgagccgtgaagtgggtgttcgcccgttttgggtgggcctgttctatacggttaacgcagtcgctggtattatcgtttccctgctgctggccaaacgttcagataatcagggcgaccgtcgcatgctgattctgttctgctgtgttatggcgatcgccaacgcagtcctgtttgccttcaatcgccattatctgaccctggtcattgcaggtgtgctgctgagctctatcgctagcgtggcgatgccgcagatttttgctctggcgcgtgaatacgcagatagttccgcccgcgaagcagtcatgttctcatcggtgatgcgtgcccaactgtcgctggcatgggttatcggtccgccgctgagctttgccattgcactgaactacggctttaccgcgatgttcctggtggcggccctgctgtttttcgtctgcgtggctctgatttggttcaccctgccgagcgttccgcgtgcagaaaacacggcagctgaaccgctgagtgatatctccggttggaaacaccgtgacgtgcgcatgctgtttattgcctctgttttcatgtggacctgtaatacgatgtatgttatcgatatgccgctgtacattagtatcgtcctgggtctgccggacaagctggcaggtctgctgatgggtaccgcagcaggcctggaaattccggtcatgctgctggctggtcattatgtgaaacgttttggcaagcgcccgatgatgctgctggcggttggctgcggtgtcctgttttacctgggtctggtgctgttccacggccgtacggaactgatgctgctgcagctgctgaacgctctgtttatcggcattatcgcgggcattggtatgatctggttccaagatctgatgccgggtcgtccgggttctgcaaccacgctgtttaccaatagcatttctacgggtgtgatcctggcaggtgtgctgcagggcgttatggccgaaacctttggccatcacgcagtctattggctggcttccctgctggcgctgatttctttcgctctgagttggcaagttcgtgaagcgcgcacggtgaagagtgttccgctggcctaataaatcgatactagcataaccccttggggcctctaaacgcgtcgacacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcatgtatgaatatcctccttagttcctattccgaagttcctattctctagaaagtataggaacttcggcgcgtcctacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagatgcttaatgaatacaacagtactgcgatgagtggcagggcggggcgtaaggcgcgccatttaaatgaagttcctattccgaagttcctattctctagaaagtataggaacttcgaagcagctccagcctacacaatcgctcaagacgtgtaatgctgcaatctgcatgcaagcttggcactggccacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggggggccatcgatgcaggtggcacttttcggggaaatgtgcgcggaacccctatttgtttatttttctaaatacattcaaatatgtatccgctcatgagacaataaccctgctgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 27 - DNA, 6684 bp, synthetic construct
  misc_feature 1..6684: note = construct pINT-ydeA
  source 1..6684: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgaagatcctttgatcttttctacggggtctgacgctcagtggaacgaaaactcacgttaagggattttggtcatgagattatcaaaaaggatcttcacctagatccttttaaactagtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccactgcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaaacgagacatactcttcctttttcaatattattgaagcatttatcagggttattgtctcatgagcggatacatatttgaatgtatttagaaaaataaacaaataggggttccgcgcacatttccccgaaaagtgccacctgaaattggccagatgattaattcctaatttttgttgacactctatcattgatagagttattttaccactccctatcagtgatagagaaaagtgaaatgaatagttcgacaaaaatctagaaataattttgtttaactttaagaaggagatatacaagagctcgagtcgaaggagatagaaccatgacaacaaacactgtttcccgcaaagtggcgtggctacgggtcgttacgctggcagtcgccgccttcatcttcaacaccaccgaatttgtccctgttggcctgctctctgacattgcgcaaagttttcacatgcaaaccgctcaggtcggcatcatgttgaccatttacgcatgggtagtagcgctaatgtcattgccttttatgttaatgaccagtcaggttgaacggcgcaaattactgatctgcctgtttgtggtgtttattgccagccacgtactgtcgtttttgtcgtggagctttaccgttctggtgatcagtcgcattggtgtggcttttgcacatgcgattttctggtcgattacggcgtctctggcgatccgtatggctccggccgggaagcgagcacaggcattgagtttaattgccaccggtacagcactggcgatggtcttaggtttacctctcgggcgcattgtgggccagtatttcggttggcgaatgaccttcttcgcgattggtattggggcgcttatcacccttttgtgcctgattaagttacttcccttactgcccagtgagcattccggttcactgaaaagcctcccgctattgttccgccgcccggcattgatgagcatttatttgttaactgtggtggttgtcaccgcccattacacggcatacagctatatcgagccttttgtacaaaacattgcgggattcagcgccaactttgccacggcattactgttattactcggtggtgcgggcattattggcagcgtgattttcggtaaactgggtaatcagtatgcgtctgcgttggtgagtacggcgattgcgctgttgctggtgtgcctggcattgctgttacctgcggcgaacagtgaaatacacctcggggtgctgagtattttctgggggatcgcgatgatgatcatcgggcttggtatgcaggttaaagtgctggcgctggcaccagatgctaccgacgtcgcgatggcgctattctccggcatatttaatattggaatcggggcgggtgcgttggtaggtaatcaggtgagtttgcactggtcaatgtcgatgattggttatgtgggcgcggtgcctgcttttgccgcgttaatttggtcaatcattatatttcgccgctggccagtgacactcgaagaacagacgcaatagtaaatcgatactagcataaccccttggggcctctaaacgcgtcgacacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcatgtatgaatatcctccttagttcctattccgaagttcctattctctagaaagtataggaacttcggcgcgtcctacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagatgcttaatgaatacaacagtactgcgatgagtggcagggcggggcgtaaggcgcgccatttaaatgaagttcctattccgaagttcctattctctagaaagtataggaacttcgaagcagctccagcctacacaatcgctcaagacgtgtaatgctgcaatctgcatgcaagcttggcactggccacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggggggccatcgatgcaggtggcacttttcggggaaatgtgcgcggaacccctatttgtttatttttctaaatacattcaaatatgtatccgctcatgagacaataaccctgctgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 28 - DNA, 6738 bp, synthetic construct
  misc_feature 1..6738: note = construct pINT-propP2
  source 1..6738: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgaagatcctttgatcttttctacggggtctgacgctcagtggaacgaaaactcacgttaagggattttggtcatgagattatcaaaaaggatcttcacctagatccttttaaactagtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccactgcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaaacgagacatactcttcctttttcaatattattgaagcatttatcagggttattgtctcatgagcggatacatatttgaatgtatttagaaaaataaacaaataggggttccgcgcacatttccccgaaaagtgccacctgaaattggccagatgattaattcctaatttttgttgacactctatcattgatagagttattttaccactccctatcagtgatagagaaaagtgaaatgaatagttcgacaaaaatctagaaataattttgtttaactttaagaaggagatatacaagagctcgagtcgaaggagatagaaccatgaatgaatccgccgtgaaaatcaaccgcacctttatctcccactatgctctgctgctgaaactgatgaccatgttcgtccaacaacagaacagtaccccgtccaatattgtggcgtttaacttcctgctgatcgcctttctgacgggtattgcgagcgccttccagaccccgacgctgtcactgtatctgtcgcaagaaatcaatgttagtccgtttttcgttggtctgttttactccgttaacgcgattatcggcattatcctgagccagattctggccaaatattctgataagcaagatgaccgtcgcaaagtcatgattgtgtgctgtctgatcgcagtgctgggttgcctgatctttgcttacagccgtaattattacgttctgattatcattggcaccacgctgctgggcctgggtagctctgcaaacccgcagtcatttgcactggctcgtgaatatgcagaaagttcccatcgcgaagctgttatgttcaccacgattatgcgcacccagatcagtctggcatggattgtcggtccgccgctgtcctttttcattgctctgaattggggctttgattatatgtacctggtcgcaggttcagctttcctgctgtgcgccggcgtgtcgaaactgctgccgaagatcccgcgtcagtctgcagtcaaaaatcaagaaattctggacaacaccccgccgcgtcgcagtgtgatttacctgtttatcgccaatctgctgctgtggacgtgtaattccatgtacctgatcaacatgccgctgttcgtgattaacgaactgcacctgggtaaagaactggcaggtaccctgatgggtacggcagcaggcctggaaattccggtgatgatctttgccggctatctgaccaaatacttctcaaaaaagcgcctgatgatgattgcactggtttcgggtctggctttttattcatcgctgctgttcagcgatcagacctggcaactgatcggcctgcagatgctgaacgcgatctttattggtatcaccgccacgattggcatggtttatttccaagacctgatgccgaccaaaatgggtacggcgaccacgctgtttagtaatgcagctaagagctcttggatcattggcggtccgatcgcgggcatcattgccgaaatctggcattacaactctgtgttttatgtggcggttgccctgattttcatcagcgtcggctgtatgtggaaggttaagtctgtctaataaatcgatactagcataaccccttggggcctctaaacgcgtcgacacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcatgtatgaatatcctccttagttcctattccgaagttcctattctctagaaagtataggaacttcggcgcgtcctacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagatgcttaatgaatacaacagtactgcgatgagtggcagggcggggcgtaaggcgcgccatttaaatgaagttcctattccgaagttcctattctctagaaagtataggaacttcgaagcagctccagcctacacaatcgctcaagacgtgtaatgctgcaatctgcatgcaagcttggcactggccacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggggggccatcgatgcaggtggcacttttcggggaaatgtgcgcggaacccctatttgtttatttttctaaatacattcaaatatgtatccgctcatgagacaataaccctgctgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 29 - DNA, 6513 bp, synthetic construct
  misc_feature 1..6513: note = construct pINT-Pc-setA
  source 1..6513: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgaagatcctttgatcttttctacggggtctgacgctcagtggaacgaaaactcacgttaagggattttggtcatgagattatcaaaaaggatcttcacctagatccttttaaactagtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccactgcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaaacgagacatactcttcctttttcaatattattgaagcatttatcagggttattgtctcatgagcggatacatatttgaatgtatttagaaaaataaacaaataggggttccgcgcacatttccccgaaaagtgccacctgaaattggccagatgattaattcctaatttttgttgacactctatcattgatagagttattttaccactccctatcagtgatagagaaaagtgaaatgaatagttcgacaaaaatctagaaataattttgtttaactttaagaaggagatatacaagagctcgagtcgaaggagatagaaccatgttctacacgggctcggcagttattggtatcgtcgtctcgcagatgctggctacgcgctcggatcgtcagggtgaccgcaagtcgctgatcttcgtttgctgtctgctgggtgcgctggcctgcatgctgtttgcgtggaaccgcaattatttcatcctgctgtttattggtgtgctgctgagctctttcggcagtaccgccaacccgcagctgtttgcactggctcgcgaacatgcagataaaacgggtcgtgaagcggccatgttcagttccatcctgcgtgcccaaatttccctggcatgggtggttggtccgccgattgcgtttgccctggcactgggcttcggttttaccacgatgtacctgaccgcagctgtcgtgttcatcctgtgtggtattctggtgaagctgtttctgccgagcatgccgaaagccgttgaaaagaccacgagcaccctggaatctccgcgtcgcaatcgtcgcgacacgctgctgctgtttgttgcgtgcaccctgatgtggacgtgtaacggcatctatctgattaatatgccgctgtacctggttcatgaactgcacctgccggaaaaactggcaggtatcatgatgggtgtcgcagcaggtctggaaatcccggttatgctgattgccggttatgtcgcaaaacgtttcggcaagcgctttctgatgcgtctggctgtcgcgagcggtctgctgtttttcggcggtctgctggtgctggatggcgaaatcgccctgctggcactgcaggctctgaacgcgattttcatcggcattctggctggcattggtatgctgtactttcaggacctgatgccgggccaagcaggtgcagctaccacgctgtttaccaacaccacgcgcgtgggttggattatctcaggttcgctggctggcatcgtggcggaaatttggaattatcacgctgtgtttttctttgcgctgctgatgatcgtcggctctatttactgcatgtggcgtattaaagatgcgtaataaatcgatactagcataaccccttggggcctctaaacgcgtcgacacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcatgtatgaatatcctccttagttcctattccgaagttcctattctctagaaagtataggaacttcggcgcgtcctacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagatgcttaatgaatacaacagtactgcgatgagtggcagggcggggcgtaaggcgcgccatttaaatgaagttcctattccgaagttcctattctctagaaagtataggaacttcgaagcagctccagcctacacaatcgctcaagacgtgtaatgctgcaatctgcatgcaagcttggcactggccacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggggggccatcgatgcaggtggcacttttcggggaaatgtgcgcggaacccctatttgtttatttttctaaatacattcaaatatgtatccgctcatgagacaataaccctgctgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 30 - DNA, 6810 bp, synthetic construct
  misc_feature 1..6810: note = construct pINT-fucP
  source 1..6810: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgaagatcctttgatcttttctacggggtctgacgctcagtggaacgaaaactcacgttaagggattttggtcatgagattatcaaaaaggatcttcacctagatccttttaaactagtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccactgcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaaacgagacatactcttcctttttcaatattattgaagcatttatcagggttattgtctcatgagcggatacatatttgaatgtatttagaaaaataaacaaataggggttccgcgcacatttccccgaaaagtgccacctgaaattggccagatgattaattcctaatttttgttgacactctatcattgatagagttattttaccactccctatcagtgatagagaaaagtgaaatgaatagttcgacaaaaatctagaaataattttgtttaactttaagaaggagatatacaagagctcgagtcgaaggagatagaaccatgggaaacacatcaatacaaacgcagagttaccgtgcggtagataaagatgcagggcaaagcagaagttacattattccattcgcgctgctgtgctcactgttttttctttgggcggtagccaataaccttaacgacattttattacctcaattccagcaggcttttacgctgacaaatttccaggctggcctgatccaatcggccttttactttggttatttcattatcccaatccctgctgggatattgatgaaaaaactcagttataaagcagggattattaccgggttatttttatatgccttgggtgctgcattattctggcccgccgcagaaataatgaactacaccttgtttttagttggcctatttattattgcagccggattaggttgtctggaaactgccgcaaacccttttgttacggtattagggccggaaagtagtggtcacttccgcttaaatcttgcgcaaacatttaactcgtttggcgcaattatcgcggttgtctttgggcaaagtcttattttgtctaacgtgccacatcaatcgcaagacgttctcgataaaatgtctccagagcaattgagtgcgtataaacacagcctggtattatcggtacagacaccttatatgatcatcgtggctatcgtgttactggtcgccctgctgatcatgctgacgaaattcccggcattgcagagtgataatcacagtgacgccaaacaaggatcgttctccgcatcgctttctcgcctggcgcgtattcgccactggcgctgggcggtattagcgcaattctgctatgtcggcgcacaaacggcctgctggagctatttgattcgctacgctgtagaagaaattccaggtatgactgcaggctttgccgctaactatttaaccggaaccatggtgtgcttctttattggtcgtttcaccggtacctggctcatcagtcgcttcgcaccacacaaagtcctggccgcctacgcattaatcgctatggcactgtgcctgatctcagccttcgctggcggtcatgtgggcttaatagccctgactttatgcagcgcctttatgtcgattcagtacccaacaatcttctcgctgggcattaagaatctcggccaggacaccaaatatggttcgtccttcatcgttatgaccattattggcggcggtattgtcactccggtcatgggttttgtcagtgacgcggcgggcaacatccccactgctgaactgatccccgcactctgcttcgcggtcatctttatctttgcccgtttccgttctcaaacggcaactaactgataaatcgatactagcataaccccttggggcctctaaacgcgtcgacacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcatgtatgaatatcctccttagttcctattccgaagttcctattctctagaaagtataggaacttcggcgcgtcctacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagatgcttaatgaatacaacagtactgcgatgagtggcagggcggggcgtaaggcgcgccatttaaatgaagttcctattccgaagttcctattctctagaaagtataggaacttcgaagcagctccagcctacacaatcgctcaagacgtgtaatgctgcaatctgcatgcaagcttggcactggccacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggggggccatcgatgcaggtggcacttttcggggaaatgtgcgcggaacccctatttgtttatttttctaaatacattcaaatatgtatccgctcatgagacaataaccctgctgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 31 - DNA, 6933 bp, synthetic construct
  misc_feature 1..6933: note = construct pINT-mdeA
  source 1..6933: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgaagatcctttgatcttttctacggggtctgacgctcagtggaacgaaaactcacgttaagggattttggtcatgagattatcaaaaaggatcttcacctagatccttttaaactagtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccactgcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaaacgagacatactcttcctttttcaatattattgaagcatttatcagggttattgtctcatgagcggatacatatttgaatgtatttagaaaaataaacaaataggggttccgcgcacatttccccgaaaagtgccacctgaaattggccagatgattaattcctaatttttgttgacactctatcattgatagagttattttaccactccctatcagtgatagagaaaagtgaaatgaatagttcgacaaaaatctagaaataattttgtttaactttaagaaggagatatacaagagctcgagtcgaaggagatagaaccatgtccaaaaaacaaaaactgacgatgattattacgatgctgatgggtggcttcttcggtctgctgaatgaaacgctgctggtgacggcactgccgagcatcatgaaagacttcgaaatttcttatacgcaggttcaatggctgaccacggcatttctgctgaccaacggcatcgttattccgctgtcagctctggtcattcagcgttacaccacgcgccaagttttcctggtcggtatctctattttctttctgggcacgctgctgtcaggtctgtcgccgcattttgcgaccctgctggttgcgcgtattatccaggcactgggcgctggtatcatgatgccgctgatgatgaccacgattctggatgtcttccaaccgcacgaacgcggcaaatatatgggcatttttggtctggtgatcggtctggcaccggcaatcggtccgaccctgagtggttatctggttgaatacttcaactggcgttccctgtttcatgtggttgcgccgatcgcggccgttacctttctgattggcttcaaaacgatcaaaaatgtgggtaccacgattaaagttccgatcgactttatttcagtcatcttctcggtgctgggctttggcggtctgctgtatggtaccagctctatttcagaaaaaggcttcgataatccgatcgtcctggtgtcgatgattggcggtgtcgtgctggttgcactgtttgtcctgcgtcagtaccgcctgagcaccccgctgctgaacttcgctgtgttcaaaaacaaacaattcaccgttggcattatcattatgggtgtgacgatggttagcatgatcggctctgaaaccattctgccgatctttgttcagaacctgctgcatcgtagtgcactggactccggtctgacgctgctgccgggtgcaattgtgatggccttcatgagcatgacctctggcgccctgtatgaaaaatttggtccgcgcaatctggcactggtgggtatggctattgttgtcatcaccacggcatattttgtggttatggatgaacagaccagtacgattatgctggcaaccgtctacgctattcgcatggtgggcatcgcgctgggtctgattccggttatgacccatacgatgaaccagctgaaaccggaaatgaatgcgcacggcagttccatgaccaacacggtgcagcaaattgccggcagcatcggtaccgcagctctgatcacgattctgagtcacgcctccaaaaacttttcaccgaccatgtcggattacaacggtatgaacaaaatcgacatgatgaaccagatcaaagtcgataccatgctgcatggctaccacgcgggttttctgttcgccctgctgattaccgtggtgtcgttcttctgttcatttatgctgcaaggcaaaaagaaagaagtggattcccgccagtaataaatcgatactagcataaccccttggggcctctaaacgcgtcgacacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcatgtatgaatatcctccttagttcctattccgaagttcctattctctagaaagtataggaacttcggcgcgtcctacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagatgcttaatgaatacaacagtactgcgatgagtggcagggcggggcgtaaggcgcgccatttaaatgaagttcctattccgaagttcctattctctagaaagtataggaacttcgaagcagctccagcctacacaatcgctcaagacgtgtaatgctgcaatctgcatgcaagcttggcactggccacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggggggccatcgatgcaggtggcacttttcggggaaatgtgcgcggaacccctatttgtttatttttctaaatacattcaaatatgtatccgctcatgagacaataaccctgctgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 32 - DNA, 7248 bp, synthetic construct
  misc_feature 1..7248: note = construct pINT-lmrA
  source 1..7248: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgaagatcctttgatcttttctacggggtctgacgctcagtggaacgaaaactcacgttaagggattttggtcatgagattatcaaaaaggatcttcacctagatccttttaaactagtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccactgcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaaacgagacatactcttcctttttcaatattattgaagcatttatcagggttattgtctcatgagcggatacatatttgaatgtatttagaaaaataaacaaataggggttccgcgcacatttccccgaaaagtgccacctgaaattggccagatgattaattcctaatttttgttgacactctatcattgatagagttattttaccactccctatcagtgatagagaaaagtgaaatgaatagttcgacaaaaatctagaaataattttgtttaactttaagaaggagatatacaagagctcgagtcgaaggagatagaaccatggcgaaccgtatcgaaggcaaagctgtggacaaaacctcaatcaaacatttcattaaactgatccgtgccgcaaaaccgcgttacctgtttttcattatcggtattctggcgggtatcgtgggcaccctgattcagctgcaagtcccgaaaatggtgcagccgctggttaactcttttggtcatggcgttaatggcggtaaagttgccctggtcattgcactgtatatcggtagtgcagcagtctccgcaattgcagctatcgtgctgggtatctttggcgaaagcgtggttaaaaacctgcgtacgcgcgtttgggataaaatgattcacctgccggtgaaatacttcgacgaagttaaaaccggtgaaatgagctctcgtctggcgaatgataccacgcaagtgaaaaacctgattgcaaatagcatcccgcaggcttttacgtctattctgctgctggtcggcagtatcgtgttcatgctgcagatgcaatggcgcctgaccctggctatgattatcgcggttccggtcgtgatgctgattatgtttccgatcatgacgttcggtcagaaaattggccgtacccgccaagatagcctggcgaactttcagggtattgcctcagaatcgctgagcgaaatccgtctggtgaaaagttccaatgccgaaaaacaggcatccaaaaaagctgaaaacgacgttaatgcactgtataaaattggtgtcaaagaagcgatctttgatggcctgatgagtccggtcatgatgctgtccatgatgctgatgatcttcggtctgctggcctatggcatttacctgatcagcacgggtgtgatgtctctgggtaccctgctgggcatgatgatgtacctgatgaacctgattggcgcggtgccgaccgttgccacgtttttcaccgaactggcgaaagcctctggtagtacgggccgtctgaccgaactgctggatgaagaacaggaagttctgcatcagggtgaatcgctggatctggaaggcaaaaccctgagcgcacgtcacgtcgactttgcttatgatgactctgaacaaattctgcgcgatatctcctttgaagcgcagccgaattcaattatcgcattcgctggcccgagtggcggtggcaaatcaaccatcttttcgctgctggaacgcttctaccaaccgacggccggtgaaattaccatcgatggccagccgattgacaacatctcactggaaaattggcgttcgcagattggtttcgttagccaagactctgctattatggcgggcacgatccgcgaaaacctgacctatggtctggaaggcgattacacggatgaagacctgtggcaggtcctggacctggcgtttgcccgttcattcgtggaaaacatgccggatcagctgaataccgaagttggtgaacgcggcgtcaaaatttcgggtggccagcgtcaacgcctggcaatcgctcgtgcgtttctgcgcaatccgaaaattctgatgctggatgaagccaccgcatctctggactccgaatcagaatcgatggtgcagaaagcgctggatagtctgatgaaaggtcgtaccacgctggtgattgcccatcgcctgtccacgatcgttgatgcagacaaaatctacttcatcgaaaaaggccagatcaccggtagcggcaaacacaacgaactggtcgcaacccacccgctgtacgcaaaatatgtctcggaacaactgacggtcggccaataataaatcgatactagcataaccccttggggcctctaaacgcgtcgacacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcatgtatgaatatcctccttagttcctattccgaagttcctattctctagaaagtataggaacttcggcgcgtcctacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagatgcttaatgaatacaacagtactgcgatgagtggcagggcggggcgtaaggcgcgccatttaaatgaagttcctattccgaagttcctattctctagaaagtataggaacttcgaagcagctccagcctacacaatcgctcaagacgtgtaatgctgcaatctgcatgcaagcttggcactggccacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggggggccatcgatgcaggtggcacttttcggggaaatgtgcgcggaacccctatttgtttatttttctaaatacattcaaatatgtatccgctcatgagacaataaccctgctgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 33 - DNA, 6684 bp, synthetic construct
  misc_feature 1..6684: note = construct pINT-Ps-setA
  source 1..6684: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgaagatcctttgatcttttctacggggtctgacgctcagtggaacgaaaactcacgttaagggattttggtcatgagattatcaaaaaggatcttcacctagatccttttaaactagtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccactgcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaaacgagacatactcttcctttttcaatattattgaagcatttatcagggttattgtctcatgagcggatacatatttgaatgtatttagaaaaataaacaaataggggttccgcgcacatttccccgaaaagtgccacctgaaattggccagatgattaattcctaatttttgttgacactctatcattgatagagttattttaccactccctatcagtgatagagaaaagtgaaatgaatagttcgacaaaaatctagaaataattttgtttaactttaagaaggagatatacaagagctcgagtcgaaggagatagaaccatgaacgaaagccagagctctcatggcggttcatggctgtcggttattgcgctggccctggcggcctttatcttcaataccacggaattcgttccggtcgcgctgctgtcagatattggccgttcgtttgacatgccgccatcacaagtgggtctgatgctgaccatctatgcgtgggtggttgccctgatgtcgctgccgatgatgctgctgacccgcaacgtcgaacgtcgcacgctgctgatttttgtgttcgtcgtgttcatcggcagtcatctggtgagttccgtggcgtcatcgtttagcatgctgatgatttctcgtattggtatcgcactgtcccacgctgtgttttggagtatcaccgcatccctggctgtgcgtgttgcaccggctggtaaacaggcccaggcactgggtctgctggcaaccggttcagcactggctatggtcctgggtattccgctgggccgtgttgtcggtgaactgctggattggcgcaccacgttcctgagcattgccatcgtggcagctctggtggttctgtgtctggcacgtaccctgccgctgctgccgagtcagaatagtggttccctgcgttccctgccgatgctgtttaaacgtccggcgctggttgcggcatatgttctgaccgccctggttattacggcgcagtttaccgcctatacgtacattgaaccgttcgcacaaaccatcgctcatctgtctggcaacatgaccacggcactgctgctgctgtttggcggtgctggtattctgggcacggtgctgttcagccgttattctaatcgctacccgaaaggttttctgatcgcagctattagtatcatggcaatgtgtctgctgctgctgctgccggcctcccgcgatagctctctgctggccgccctggtcgtggtttggggtattgcgggcatgtgtttcggcctggcgctgcaggccaaagttctgaacctggcaagcgatgctaccgacgtcgcgatggccctgttttctggcatttataatgttggtatcggcggtggcgccctgctgggttcactggttacggcacacctgggcctgtcggacgttggtattgtcggtggcctgctggccctgagcggcgtcgtgctgtgctgttttgccacctatcgctttgcacgtccggtgggttctgcagctctgtaataaatcgatactagcataaccccttggggcctctaaacgcgtcgacacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcatgtatgaatatcctccttagttcctattccgaagttcctattctctagaaagtataggaacttcggcgcgtcctacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagatgcttaatgaatacaacagtactgcgatgagtggcagggcggggcgtaaggcgcgccatttaaatgaagttcctattccgaagttcctattctctagaaagtataggaacttcgaagcagctccagcctacacaatcgctcaagacgtgtaatgctgcaatctgcatgcaagcttggcactggccacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggggggccatcgatgcaggtggcacttttcggggaaatgtgcgcggaacccctatttgtttatttttctaaatacattcaaatatgtatccgctcatgagacaataaccctgctgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 34 - DNA, 6692 bp, synthetic construct
  misc_feature 1..6692: note = construct pINT-Bb-setA
  source 1..6692: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgaagatcctttgatcttttctacggggtctgacgctcagtggaacgaaaactcacgttaagggattttggtcatgagattatcaaaaaggatcttcacctagatccttttaaactagtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccactgcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaaacgagacatactcttcctttttcaatattattgaagcatttatcagggttattgtctcatgagcggatacatatttgaatgtatttagaaaaataaacaaataggggttccgcgcacatttccccgaaaagtgccacctgaaattggccagatgattaattcctaatttttgttgacactctatcattgatagagttattttaccactccctatcagtgatagagaaaagtgaaatgaatagttcgacaaaaatctagaaataattttgtttaactttaagaaggagatatacaagagctcgagtcgaaggagatagaaccatgaccattgcaacggtttcccgcaaaaccgcttggctgcgtgtggttaccctggccgttgccgcctttatcttcaacaccacggaatttgtcccggtgggcctgctgagtgatattgcgcagtccttcggcatggaaaccgcccaagtgggtattatgctgacgatctatgcctgggttgtggcactgatgagcctgccgtttatgctgatgacctctcaggtggaacgtcgccgtctgctgattagcatctttctgctgttcatcgcaagtcatgttctgtcctttctggcgtggaatttcaccgttctggtcatttctcgcattggtatcgcgtttgcccacgcaattttctggtcaatcacggcttcgctggcgattcgtatggctccggcgggcaagaaagcgcaggcactgagtctgctggcgaccggtacggctctggcgatggttctgggtctgccgatcggccgcattgtcggtcaatactttggctggcgtaccacgtttttcgtgattggcgttgtcgcagctatcaccctgttctgcctgattaaactgctgccgaaactgccgagcgaacatagtggttccctgagctctgtgccgaaactgtttcgccgtccggcgctggttaacatctatgccctgattgcaatcgtggttaccgcacactacacggcttatagttacatcgaaccgttcgtgcagcaaattgccggcctgtccgctaactttgcgaccctgctgctgctgctgtttggcggtgcgggtattatcggctctgttctgtttggtaaatggggcaataaacatgccagcggtctggtctctggcgccattgcactgatggccgcatgtctggtgctgctgctgccggcagctcagggtgaactgaccctggccggcctgtcactgttttggggtatttcgatcatgattgtcgcactgggtatgcaagtgaaagttctggctctggccccggatgccaccgatgttgccatgagcctgttttctggcatcttcaacatcggcattggtgccggcgcactgctgggtaatcaggtgtcactgcacatttcaatgtcggacatcggttttattggcgccatcccggcaattatcgctctggtctggtcgattctggtgtccgccgttggccggttgccctggaagaacatccgcaggcaacccactaataaatcgatactagcataaccccttggggcctctaaacgcgtcgacacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcatgtatgaatatcctccttagttcctattccgaagttcctattctctagaaagtataggaacttcggcgcgtcctacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagatgcttaatgaatacaacagtactgcgatgagtggcagggcggggcgtaaggcgcgccatttaaatgaagttcctattccgaagttcctattctctagaaagtataggaacttcgaagcagctccagcctacacaatcgctcaagacgtgtaatgctgcaatctgcatgcaagcttggcactggccacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggggggccatcgatgcaggtggcacttttcggggaaatgtgcgcggaacccctatttgtttatttttctaaatacattcaaatatgtatccgctcatgagacaataaccctgctgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 35 - DNA, 35 bp, synthetic construct
  misc_feature 1..35: note = Primer
  source 1..35: mol_type = other DNA
ttactcagcaataaactgatattccgtcaggctgg

SEQ ID NO: 36 - DNA, 83 bp, synthetic construct
  misc_feature 1..83: note = Primer
  source 1..83: mol_type = other DNA
ttgtaatctcgcgctcttcacatcagactttccatatagagcgtaatttccgttaacgtcggtagtgctgaccttgccggagg

SEQ ID NO: 37 - DNA, 53 bp, synthetic construct
  misc_feature 1..53: note = Primer
  source 1..53: mol_type = other DNA
ctgtctcttatcacatctcctgaaatggccagatgtaattcctaatttttgtt

SEQ ID NO: 38 - DNA, 44 bp, synthetic construct
  misc_feature 1..44: note = Primer
  source 1..44: mol_type = other DNA
ctgtctcttatcacatctcacattacatctgagcgattgttagg

SEQ ID NO: 39 - DNA, 30 bp, synthetic construct
  misc_feature 1..30: note = Primer
  source 1..30: mol_type = other DNA
gatcacatatgagagttctggttaccggtg

SEQ ID NO: 40 - DNA, 40 bp, synthetic construct
  misc_feature 1..40: note = Primer
  source 1..40: mol_type = other DNA
gatcactcgagtcattaatcgggatatccctgtggatggc

SEQ ID NO: 41 - DNA, 60 bp, synthetic construct
  misc_feature 1..60: note = Primer
  source 1..60: mol_type = other DNA
gtcgatgaagccctgaaagacgcgcagactatgcacttcattgaaaacaaaaacttcgtc

SEQ ID NO: 42 - DNA, 60 bp, synthetic construct
  misc_feature 1..60: note = Primer
  source 1..60: mol_type = other DNA
gatggcctttttgcgtgtcgacgcggccgcctagataaacaggatgatatttttgccttg

SEQ ID NO: 43 - DNA, 60 bp, synthetic construct
  misc_feature 1..60: note = Primer
  source 1..60: mol_type = other DNA
caaggcaaaaatatcatcctgtttatctaggcggccgcgtcgacacgcaaaaaggccatc

SEQ ID NO: 44 - DNA, 60 bp, synthetic construct
  misc_feature 1..60: note = Primer
  source 1..60: mol_type = other DNA
gacgaagtttttgttttcaatgaagtgcatagtctgcgcgtctttcagggcttcatcgac

SEQ ID NO: 45 - DNA, 60 bp, synthetic construct
  misc_feature 1..60: note = Primer
  source 1..60: mol_type = other DNA
atggcctttttgcgtgtcgacgcggccgcttaattcgagcgggtaaagatcttcatcagg

SEQ ID NO: 46 - DNA, 60 bp, synthetic construct; identical to SEQ ID NO: 45
  misc_feature 1..60: note = Primer
  source 1..60: mol_type = other DNA

SEQ ID NO: 47 - DNA, 60 bp, synthetic construct
  misc_feature 1..60: note = Primer
  source 1..60: mol_type = other DNA
ctgatgaagatctttacccgctcgaattaagcggccgcgtcgacacgcaaaaaggccatc

SEQ ID NO: 48 - DNA, 60 bp, synthetic construct
  misc_feature 1..60: note = Primer
  source 1..60: mol_type = other DNA
cggggtacgcttaatcaggttatcaatcatagtctgcgcgtctttcagggcttcatcgac

SEQ ID NO: 49 - DNA, 60 bp, synthetic construct
  misc_feature 1..60: note = Primer
  source 1..60: mol_type = other DNA
gtcgatgaagccctgaaagacgcgcagactatgagcggtgaacactatgtcattagcctg

SEQ ID NO: 50 - DNA, 60 bp, synthetic construct
  misc_feature 1..60: note = Primer
  source 1..60: mol_type = other DNA
gatggcctttttgcgtgtcgacgcggccgctcatttaaattcgatgatcatcttgtcgtt

SEQ ID NO: 51 - DNA, 60 bp, synthetic construct
  misc_feature 1..60: note = Primer
  source 1..60: mol_type = other DNA
aacgacaagatgatcatcgaatttaaatgagcggccgcgtcgacacgcaaaaaggccatc

SEQ ID NO: 52 - DNA, 60 bp, synthetic construct
  misc_feature 1..60: note = Primer
  source 1..60: mol_type = other DNA
caggctaatgacatagtgttcaccgctcatagtctgcgcgtctttcagggcttcatcgac

SEQ ID NO: 53 - DNA, 60 bp, synthetic construct
  misc_feature 1..60: note = Primer
  source 1..60: mol_type = other DNA
gtcgatgaagccctgaaagacgcgcagactatggatgaaatcaaactgtcggtggttatg

SEQ ID NO: 54 - DNA, 60 bp, synthetic construct
  misc_feature 1..60: note = Primer
  source 1..60: mol_type = other DNA
gatggcctttttgcgtgtcgacgcggccgctcattggcgacgccaatcgaacgcaacgcg

SEQ ID NO: 55 - DNA, 60 bp, synthetic construct
  misc_feature 1..60: note = Primer
  source 1..60: mol_type = other DNA
cgcgttgcgttcgattggcgtcgccaatgagcggccgcgtcgacacgcaaaaaggccatc

SEQ ID NO: 56 - DNA, 60 bp, synthetic construct
  misc_feature 1..60: note = Primer
  source 1..60: mol_type = other DNA
cataaccaccgacagtttgatttcatccatagtctgcgcgtctttcagggcttcatcgac

SEQ ID NO: 57 - DNA, 60 bp, synthetic construct
  misc_feature 1..60: note = Primer
  source 1..60: mol_type = other DNA
gtcgatgaagccctgaaagacgcgcagactatggaaaactatgtcgtctctatccgcacc

SEQ ID NO: 58 - DNA, 60 bp, synthetic construct
  misc_feature 1..60: note = Primer
  source 1..60: mol_type = other DNA
gatggcctttttgcgtgtcgacgcggccgctcatttgaacggaacaatctttttgtcatc

SEQ ID NO: 59 - DNA, 60 bp, synthetic construct
  misc_feature 1..60: note = Primer
  source 1..60: mol_type = other DNA
gatgacaaaaagattgttccgttcaaatgagcggccgcgtcgacacgcaaaaaggccatc

SEQ ID NO: 60 - DNA, 60 bp, synthetic construct
  misc_feature 1..60: note = Primer
  source 1..60: mol_type = other DNA
ggtgcggatagagacgacatagttttccatagtctgcgcgtctttcagggcttcatcgac

SEQ ID NO: 61 - DNA, 60 bp, synthetic construct
  misc_feature 1..60: note = Primer
  source 1..60: mol_type = other DNA
gtcgatgaagccctgaaagacgcgcagactatgtcctcagctttccattacgtcattagc

SEQ ID NO: 62 - DNA, 60 bp, synthetic construct
  misc_feature 1..60: note = Primer
  source 1..60: mol_type = other DNA
gatggcctttttgcgtgtcgacgcggccgctcattcaaattcgataatcatggtgatttt

SEQ ID NO: 63 - DNA, 60 bp, synthetic construct
  misc_feature 1..60: note = Primer
  source 1..60: mol_type = other DNA
aaaatcaccatgattatcgaatttgaatgagcggccgcgtcgacacgcaaaaaggccatc

SEQ ID NO: 64 - DNA, 60 bp, synthetic construct
  misc_feature 1..60: note = Primer
  source 1..60: mol_type = other DNA
gctaatgacgtaatggaaagctgaggacatagtctgcgcgtctttcagggcttcatcgac

SEQ ID NO: 65 - DNA, 60 bp, synthetic construct
  misc_feature 1..60: note = Primer
  source 1..60: mol_type = other DNA
gtcgatgaagccctgaaagacgcgcagactatgaacgtgaataagccgaccaccgaaaag

SEQ ID NO: 66 - DNA, 60 bp, synthetic construct
  misc_feature 1..60: note = Primer
  source 1..60: mol_type = other DNA
gatggcctttttgcgtgtcgacgcggccgctcagtattcttcaattttgtccagttgata

SEQ ID NO: 67 - DNA, 60 bp, synthetic construct
  misc_feature 1..60: note = Primer
  source 1..60: mol_type = other DNA
tatcaactggacaaaattgaagaatactgagcggccgcgtcgacacgcaaaaaggccatc

SEQ ID NO: 68 - DNA, 60 bp, synthetic construct
  misc_feature 1..60: note = Primer
  source 1..60: mol_type = other DNA
cttttcggtggtcggcttattcacgttcatagtctgcgcgtctttcagggcttcatcgac

SEQ ID NO: 69 - DNA, 60 bp, synthetic construct
  misc_feature 1..60: note = Primer
  source 1..60: mol_type = other DNA
gtgatcaacgccgccagcggtcgtcagactgtcgatgaagccctgaaagacgcgcagact

SEQ ID NO: 70 - DNA, 60 bp, synthetic construct
  misc_feature 1..60: note = Primer
  source 1..60: mol_type = other DNA
gcggccgcgtcgacacgcaaaaaggccatccatccgtcaggatggccttctgcttaattt

SEQ ID NO: 71 - DNA, 60 bp, synthetic construct
  misc_feature 1..60: note = Primer
  source 1..60: mol_type = other DNA
aaattaagcagaaggccatcctgacggatggatggcctttttgcgtgtcgacgcggccgc

SEQ ID NO: 72 - DNA, 60 bp, synthetic construct
  misc_feature 1..60: note = Primer
  source 1..60: mol_type = other DNA
agtctgcgcgtctttcagggcttcatcgacagtctgacgaccgctggcggcgttgatcac

SEQ ID NO: 73 - DNA, 66 bp, synthetic construct
  misc_feature 1..66: note = Primer
  source 1..66: mol_type = other DNA
gtttaactttaataaggagatataccatgctgacggaagtgcgcccggtctctacgacgaaaccgc

SEQ ID NO: 74 - DNA, 66 bp, synthetic construct
  misc_feature 1..66: note = Primer
  source 1..66: mol_type = other DNA
cgacctgcaggcgcgccgagctcgaattcatttgatgtatttgcaatagaacacagaaaagaccgt

SEQ ID NO: 75 - DNA, 66 bp, synthetic construct
  misc_feature 1..66: note = Primer
  source 1..66: mol_type = other DNA
gtgttctattgcaaatacatcaaatgaattcgagctcggcgcgcctgcaggtcgacaagcttgcgg

SEQ ID NO: 76 - DNA, 69 bp, synthetic construct
  misc_feature 1..69: note = Primer
  source 1..69: mol_type = other DNA
gagaccgggcgcacttccgtcagcatggtatatctccttattaaagttaaacaaaattatttctacagg

SEQ ID NO: 77 - DNA, 62 bp, synthetic construct
  misc_feature 1..62: note = Primer
  source 1..62: mol_type = other DNA
gtatggtgaccctgtggcgcaaatgagaattcgagctcggcgcgcctgcaggtcgacaagct

SEQ ID NO: 78 - DNA, 64 bp, synthetic construct
  misc_feature 1..64: note = Primer
  source 1..64: mol_type = other DNA
gcgctgccctgtttgattttatccatggtatatctccttattaaagttaaacaaaattatttct

SEQ ID NO: 79 - DNA, 63 bp, synthetic construct
  misc_feature 1..63: note = Primer
  source 1..63: mol_type = other DNA
cctgcaggcgcgccgagctcgaattctcatttgcgccacagggtcaccatacgtgccggcagg

SEQ ID NO: 80 - DNA, 66 bp, synthetic construct
  misc_feature 1..66: note = Primer
  source 1..66: mol_type = other DNA
gtttaactttaataaggagatataccatggataaaatcaaacagggcagcgcctctctggttgtcg

SEQ ID NO: 81 - DNA, 57 bp, synthetic construct
  misc_feature 1..57: note = Primer
  source 1..57: mol_type = other DNA
cagactcgagggtaccgacgtcctaataagtagatgaatatttatcaggacgaagat

SEQ ID NO: 82 - DNA, 55 bp, synthetic construct
  misc_feature 1..55: note = Primer
  source 1..55: mol_type = other DNA
aactaaaggtttattttccatatgtatatctccttcttatacttaactaatatac

SEQ ID NO: 83 - DNA, 64 bp, synthetic construct
  misc_feature 1..64: note = Primer
  source 1..64: mol_type = other DNA
taaatattcatctacttattaggacgtcggtaccctcgagtctggtaaagaaaccgctgctgcg

SEQ ID NO: 84 - DNA, 59 bp, synthetic construct
  misc_feature 1..59: note = Primer
  source 1..59: mol_type = other DNA
gtataagaaggagatatacatatggaaaataaacctttagtttcagttttgatttgtgc

SEQ ID NO: 85 - DNA, 75 bp, synthetic construct
  misc_feature 1..75: note = Primer
  source 1..75: mol_type = other DNA
taactttaagaaggagatatacaagagctcgagtcgaaggagatagaaccatggcaacagcatggtataaacaag

SEQ ID NO: 86 - DNA, 82 bp, synthetic construct
  misc_feature 1..82: note = Primer
  source 1..82: mol_type = other DNA
gcgtgtcgacgcgtttagaggccccaaggggttatgctagtatcgatttatcatttagccacggatagtttataaattttac

SEQ ID NO: 87 - DNA, 80 bp, synthetic construct
  misc_feature 1..80: note = Primer
  source 1..80: mol_type = other DNA
ggttctatctccttcgactcgagctcttgtatatctccttcttaaagttaaacaaaattatttctagatttttgtcgaac

SEQ ID NO: 88 - DNA, 63 bp, synthetic construct
  misc_feature 1..63: note = Primer
  source 1..63: mol_type = other DNA
taaatcgatactagcataaccccttggggcctctaaacgcgtcgacacgcaaaaaggccatcc

SEQ ID NO: 89 - DNA, 80 bp, synthetic construct
  misc_feature 1..80: note = Primer
  source 1..80: mol_type = other DNA
gttcgacaaaaatctagaaataattttgtttaactttaagaaggagatatacaagagctcgagtcgaaggagatagaacc

SEQ ID NO: 90 - DNA, 63 bp, synthetic construct
  misc_feature 1..63: note = Primer
  source 1..63: mol_type = other DNA
ggatggcctttttgcgtgtcgacgcgtttagaggccccaaggggttatgctagtatcgattta

SEQ ID NO: 91 - DNA, 34 bp, synthetic construct
  misc_feature 1..34: note = Primer
  source 1..34: mol_type = other DNA
atatgacgtctcattagcggtttttcaggagacg

SEQ ID NO: 92 - DNA, 35 bp, synthetic construct
  misc_feature 1..35: note = Primer
  source 1..35: mol_type = other DNA
atatcatatgccgtccgaagcattccgtcgtcacc

SEQ ID NO: 93 - DNA, 62 bp, synthetic construct
  misc_feature 1..62: note = Primer
  source 1..62: mol_type = other DNA
taactttaataaggagatataccatgacgcaatttaatcccgttgatcatccacatcgccgc

SEQ ID NO: 94 - DNA, 67 bp, synthetic construct
  misc_feature 1..67: note = Primer
  source 1..67: mol_type = other DNA
attttcgcgaatccggagtgtaaaagcttgcggccgcataatgcttaagtcgaacagaaagtaatcg

SEQ ID NO: 95 - DNA, 72 bp, synthetic construct
  misc_feature 1..72: note = Primer
  source 1..72: mol_type = other DNA
aagcattatgcggccgcaagcttttacactccggattcgcgaaaatggatatcgctgactgcgcgcaaacgc

SEQ ID NO: 96 - DNA, 68 bp, synthetic construct
  misc_feature 1..68: note = Primer
  source 1..68: mol_type = other DNA
tcaacgggattaaattgcgtcatggtatatctccttattaaagttaaacaaaattatttctacagggg

SEQ ID NO: 97 - DNA, 57 bp, synthetic construct
  misc_feature 1..57: note = Primer
  source 1..57: mol_type = other DNA
atggtgatggctgctgcccatttaaaccgctttgactgcgtcggcaatacggtgcgc

SEQ ID NO: 98 - DNA, 60 bp, synthetic construct
  misc_feature 1..60: note = Primer
  source 1..60: mol_type = other DNA
gtttaactttaataaggagatataccatgctgaacaacgcgatgtctgttgttatcctgg

SEQ ID NO: 99 - DNA, 53 bp, synthetic construct
  misc_feature 1..53: note = Primer
  source 1..53: mol_type = other DNA
cgcagtcaaagcggtttaaatgggcagcagccatcaccatcatcaccacagcc

SEQ ID NO: 100 - DNA, 59 bp, synthetic construct
  misc_feature 1..59: note = Primer
  source 1..59: mol_type = other DNA
tcgcgttgttcagcatggtatatctccttattaaagttaaacaaaattatttctacagg

SEQ ID NO: 101 - DNA, 35 bp, synthetic construct
  misc_feature 1..35: note = Primer
  source 1..35: mol_type = other DNA
atatatcatatgtgcggtatcgttggtgctatcgc

SEQ ID NO: 102 - DNA, 36 bp, synthetic construct
  misc_feature 1..36: note = Primer
  source 1..36: mol_type = other DNA
atatatgacgtcttattccacggtcacggatttcgc